(19) United States
(12) Patent Application Publication
(10) Pub. No.: US 2010/0179225 A1
(43) Pub. Date: Jul. 15, 2010

(54) POI Prevention

(75) Inventors: Jörg KALFF, Bonn (DE); Sven Wehner, Bonn (DE); Nico Schäfer, Koln (DE); Thais M. Sielecki-Dzurdz, Kennett Square, PA (US)

Correspondence Address: Law Office of John K. Pike, PLLC, 2121 Eisenhower Avenue, Suite 200, Alexandria, VA, 22314, US

(73) Assignees: CYTOKINE PHARMASCIENCES, INC., King of Prussia, PA (US); RHEINISCHE FRIEDRICH-WILHELMS-UNIVERSITAT BONN, Bonn (DE)

(21) Appl. No.: 12/560,402

(22) Filed: Sep. 15, 2009

Related U.S. Application Data
(60) Provisional application No. 61/195,005, filed on Sep. 19, 2008.

(51) Int. Cl. A61K 31/155; A61P 29/00; A61P 9/10; A61P 1/02

(52) U.S. Cl. 514/632

(57) ABSTRACT

A method is provided, comprising administering at least one guanylhydrazone or salt thereof or a combination thereof to a subject to prevent or ameliorate in said subject at least one of postoperative intestinal inflammation, postoperative ileus, ischemia reperfusion injury, or a combination thereof.

### CROSS-REFERENCE TO RELATED APPLICATIONS

This application is based on U.S. Provisional Application No. 61/195,005, filed Sep. 15, 2008, the entire contents of which are hereby incorporated by reference.

### BACKGROUND

1. Field of the Invention

This disclosure generally relates to postoperative intestinal inflammation reactions and complications associated therewith and compounds, compositions, and methods for prevention and treatment.

2. Discussion of the Background

Major surgery procedures cause an inflammatory reaction of the intestinal wall, in particular the intestinal muscularis. This inflammatory reaction is the result of the surgical (mechanical) trauma and its intensity corresponds to the extent of the trauma elicited by the surgery. However, every abdominal surgery, as well as other more extensive surgical procedures such as, for example, heart surgery or surgery of the thorax, will cause such postoperative inflammatory reactions of the intestinal wall (Livingston E H, Passaro E P. Postoperative ileus. 35 ed. 1990:121-132). The mere manipulation of the bowel during surgery initiates a local inflammation within the intestinal muscularis (Kalff J C, Schraut W H, Simmons R L et al. Surgical manipulation of the gut elicits an intestinal muscularis inflammatory response resulting in postsurgical ileus. Ann Surg 1998; 228:652-663).

One iatrogenic complication associated with a severe inflammation of the tunica muscularis is postoperative ileus (POI). The recovery of postoperative gastrointestinal motor function represents a crucial factor for, inter alia, the outcome of surgery, the length of hospital stay, and perioperative expenses (Prasad M, Matthews J B. Deflating postoperative ileus. Gastroenterology 1999; 117:489-492; Livingston E H, Passaro E P, Jr. Postoperative ileus. Dig Dis Sci 1990; 35:121-132). Furthermore, nausea, vomition, abdominal pain, aspiration, orthostatic dysregulation (OD) and severe complications may result. The lack of sufficient motility in the gut may also lead to an increased bacterial translocation that may ultimately result in peritonitis, a systemic inflammatory response syndrome (SIRS) or a sepsis. The postoperative inflammatory reaction generally leads to an increased mortality risk in patients.

Other iatrogenic complications associated with a severe inflammatory response are ischemia reperfusion injuries (IRI) after transplantation surgery. The absence of oxygen and nutrients from blood creates a condition in which the restoration of circulation results in inflammation and oxidative damage through the induction of oxidative stress rather than restoration of normal function.

So far, all known methods for the treatment of a postoperative intestinal inflammation are directed to the treatment of an already existent (severe) inflammatory reaction. In the last years, many selective anti-inflammatory strategies were described, wherein most of them target individual proinflammatory mediators like IL-6 (Wehner S, Schwarz N T, Hundsdoerfer R et al. Induction of IL-6 within the rodent intestinal muscularis after intestinal surgical stress. Surgery 2005; 137:436-446), adhesion molecules and chemokines like ICAM-1 (Kalff J C, Carlos T M, Schraut W H et al. Surgically induced leukocytic infiltrates within the rat intestinal muscularis mediate postoperative ileus. Gastroenterology 1999; 117:378-387; The FO, de Jonge W J, Bennink R J et al. The ICAM-1 antisense oligonucleotide ISIS-3082 prevents the development of postoperative ileus in mice. Br J Pharmacol 2005; 146:252-258), MCP-1 (Turler A, Schwarz N T, Turler E et al. MCP-1 causes leukocyte recruitment and subsequently endotoxemic ileus in rat. American Journal of Physiology—Gastrointestinal & Liver Physiology 2002; 282:G145-G155) or inhibition of leukocyte recruitment via postoperative blocking of ICAM-1/LFA-1 (WO 03/068261). Others have described inhibitory kinetic factors such as NO or prostaglandins for this purpose (Kalff J C, Schraut W H, Billiar T R et al. Role of inducible nitric oxide synthase in postoperative intestinal smooth muscle dysfunction in rodents. Gastroenterology 2000; 118:316-327; Schwarz N T, Kalff J C, Turler A et al. Prostanoid Production Via COX-2 as a Causative Mechanism of Rodent Postoperative Ileus. Gastroenterology 2001; 121:1354-1371; Kalff J C, Turler A, Schwarz N T et al. Intra-abdominal activation of a local inflammatory response within the human muscularis externa during laparotomy. Ann Surg 2003; 237:301-315) or have suggested vagal nerve stimulation by electrical stimulation or intrathecal administration of an agent such as CNI-1493 (WO 03/068261). Most of these strategies furthermore just inhibit singular proinflammatory targets of the severe and complex inflammation. Furthermore, many clinical trials of mediator-directed therapies have failed for attenuation of complex inflammation as present in severe sepsis (Marshall J C. Clinical trials of mediator-directed therapy in sepsis: what have we learned? Intensive Care Med 2000; 26 Suppl 1:S75-S83; Minnich D J, Moldawer L L. Anti-cytokine and anti-inflammatory therapies for the treatment of severe sepsis: progress and pitfalls. Proc Nutr Soc 2004; 63:437-441).

A main disadvantage of the current strategies is that the various treatments only start after the inflammatory reaction has already manifested itself. Therefore, the need for more advanced and preferably prophylactic methods for the treatment of postoperative intestinal inflammation reactions and complications associated therewith exists. The present invention provides a solution to this problem.

### BRIEF DESCRIPTION OF THE DRAWINGS

FIG. 1 is the structural formula of the free base form of an exemplary guanylhydrazone, Semapimod, of which the tetravalent HCl salt is known as CNI-1493.

FIG. 2: Shows the activation of p38-MAPK and JNK/SAPK after intestinal manipulation (IM). (A) Phosphorylation (p) of p38-MAPK was detected by immunoblotting C57BL6/J mice ME lysates immediately after IM with maximum at 15 minutes, decreasing within next 45 minutes. JNK/SAPK phosphorylation was also observed 15 minutes after IM. However, phosphorylation levels remained unaffected for at least 60 minutes postoperatively. (B) Preoperative treatment with 5 mg/kg CNI-1493 (CNI-1493, the tetravalent HCl salt of the compound shown in FIG. 1) resulted in a diminished p38-MAPK phosphorylation, whereas pJNK/SAPK levels were not affected. Unphosphorylated proteins were blotted as loading controls. Control (CTL) ME specimens were from untreated animals.

FIG. 3: Shows the phosphorylation of p38-MAPK in homozygous colony stimulating factor-1 mutant mice (op−/−) and op+/? (a mixed population of unknown heterozygous +/− or homozygous +/+ wildtype mice) after IM. Levels of p38-MAPK phosphorylation (pp 38) were determined from ME lysates by ELISA 20 minutes postoperatively. pp 38 levels were significantly increased (p<0.001) in all groups after IM compared to unoperated op−/− controls (CTL). After IM, pp 38 levels were significantly decreased in op−/− mice compared to the op+/? (i.e. a mixed population of homozygous (op+/+) and heterozygous (op+/−) mice strains) group (p<0.01), but did not differ from the CNI-1493 (5 mg/kg) (+S) treated op−/− IM group (#). Controls (CTL) were unoperated op−/− mice. Values are expressed as mean±SEM.

FIG. 4: Shows the effects of the use of a guanylhydrazone for reducing the postoperative expression of proinflammatory mediators, wherein the guanylhydrazone is to be administered prior to surgery. mRNA expression of MIP-1α(A), IL-6 (B), MCP-1 (C) and ICAM (D) was analyzed by quantitative PCR. Three groups were investigated: placebo treated sham operated mice, placebo treated IM mice and CNI-1493 (5 mg/kg i.v.) treated IM animals. All genes were significantly upregulated as early as 1 h after IM and peaked 6 h after IM, except ICAM which peaked at 3 h. CNI-1493 treatment resulted in a significant reduction of MIP-1α (3 h and 6 h), IL-6 (6 h), MCP-1 (3, 6 and 24 h) and ICAM-1 (1 h and 3 h) compared to the placebo IM group. Values are expressed as mean±SEM. Asterisks indicate statistically significant differences (**=p<0.01, ***=p<0.001) between indicated samples (n=4-5). Values are expressed as mean±SEM. Thus, FIG. 4 shows the effects of a method for reducing the postoperative expression of proinflammatory mediators, wherein a guanylhydrazone is administered prior to surgery.

FIG. 5: Is a histogram showing the effects of the use of a guanylhydrazone for the diminishing postoperative neutrophil infiltration into ME, wherein the guanylhydrazone is to be administered prior to surgery. Myeloperoxidase staining for detection of neutrophils in mouse ME whole mounts was performed 24 h after IM or laparotomy (Sham) of placebo or CNI-1493 (5 mg/kg) treated mice. Both intravenous and intraperitoneal applications of CNI-1493 resulted in a significantly diminished neutrophil infiltration compared to placebo groups. Indicated probes differ significantly (**=p<0.01, ***=p<0.001) from each other. CNI-1493 IM i.v. vs. IM i.p. group did not differ significantly (#).Values are expressed as mean±SEM; n=5-7. Thus, FIG. 5 shows the results of a method for diminishing postoperative neutrophil infiltration into ME, wherein a guanylhydrazone is administered prior to surgery.

FIG. 6: Shows the effect of the use of a guanylhydrazone for reducing the postoperative nitric oxide (NO) production in ME. Muscle specimens from untreated and IM mice were taken 24 h after operation and cultured for 24 hours. NO production in the supernatant of the cultures was determined photometrically by Griess reaction. After IM, NO production significantly increased (**=p<0.01) in the placebo group. NO production was significantly decreased in the CNI-1493 (5 mg/kg) IM group compared to the placebo IM group (**, p<0.01). After CNI-1493 treatment, the IM group did not differ significantly from unoperated controls (#). Data are expressed as mean±SEM; n=4-5. Thus, FIG. 6 shows the results of a method for reducing postoperative NO production in ME, wherein a guanylhydrazone is administered prior to surgery.

FIG. 7: Shows the effect of the use of a guanylhydrazone for the prevention of postoperative contractile impairment of jejuna smooth muscle. A measurement of jejunal smooth muscle contractility was carried out in vitro. Vital muscle specimens from controls and placebo or CNI-1493 (5 mg/kg) i.v. treated IM mice were prepared. Spontaneous and bethanechol induced muscle contractility was recorded. (A) Tracings of muscle contractility at 100 μM bethanechol stimulation within the three different groups. After placebo treatment with IM, an impaired smooth muscle function was observed. CNI-1493 treatment in vivo prevented the contractile impairment observed in vitro. (B) Bethanechol-induced smooth muscle contractility was significantly reduced (##=p<0.01, ###=p<0.001) in the placebo IM group at 1-300 μM concentration compared to controls. CNI-1493 treated IM animals did not differ significantly from unoperated controls at any concentration but from placebo IM group at 1-300 μM bethanecol (*=p<0.05, ***=p<0.001). Data are expressed as the mean±SD; n=4 for controls and 8-9 for the IM groups. Thus, FIG. 7 shows the results of a method for the prevention of postoperative contractile impairment of jejunal smooth muscle, wherein a guanylhydrazone is administered prior to surgery.

FIG. 8: Shows the effect of the use of a guanylhydrazone for the prevention of a postoperative delay in GIT and colonic transit, wherein the guanylhydrazone is administered prior to surgery. The effect of CNI-1493 on GIT (A-D) and colonic transit (E). GIT was measured as the percent of non-absorbable fluorescein-labeled dextran in 15 GI segments 90 minutes after oral ingestion. Placebo or CNI-1493 (5 mg/kg) was administered to mice via the i.v (A,B) or i.p. (C,D) route. Panels A and C show the distribution of FITC-dextran along the entire gastrointestinal tract after preoperative i.v. or i.p. drug administration, respectively. In CNI-1493 treated animals most of the marker is located in distal jejunum compared to proximal jejunal location in placebo treated animals. Calculation of the geometric center (panels B, D) demonstrated a significant delay of GIT in placebo treated animals (***=p<0.001) after IM (P-IM) compared to CNI-1493 treated groups, independent of their application routes. Geometric centers (GC) for the 15 intestinal segments stomach (Sto), small intestine (SI 1-9), cecum (Cec) and colon (Co) are displayed as mean (Sham n=5-6, IM n=9-10). Colonic transit times were displayed mean with all individual values. (n=5-6). In the i.v. but not in the i.p. route, GIT from CNI-1493 treated IM animals significantly differed (**=p<0.01) from CNI-1493 treated sham groups (S-Sham). However, in both routes it did not differ from placebo treated sham groups (P-Sham). (E) Colonic transit time showed a significant delay (***=p<0.001) in placebo treated animals (P-IM) compared to unmanipulated controls (P-CTL). This delay was significantly improved (****=p<0.001) in the CNI-1493 treated IM group (S-IM) and did not differ (#) from the respective control (S-CTL).

FIG. 9: Shows the effect of CNI-1493 on intestinal wound healing. CNI-1493 (5 mg/kg) or placebo was intravenously administered to mice 90 minutes before small bowel transection, followed by an anastomosis. (A) On indicated postoperative days (POD) hydroxyproline content of the anastomotic tissue was determined. Hydroxyproline content was significantly increased at POD 5 (**p<0.01) and 10 (***p<0.001) in the CNI-1493 group and at POD 10 in the placebo group compared to POD 2, respectively. However, both groups did not differ from each other at any POD. (B) Anastomotic bursting pressure was analyzed in placebo and CNI-1493 treated groups at indicated POD. Anastomotic strength increased in both group significantly (***=p<0.001) at POD 5 and 10 compared to POD 2, respectively. However, both groups did not differ from each other at any POD. n=8-11, data are expressed as scattered dot blot with mean.

FIG. 10: Is a histogram showing the effects of the use of a guanylhydrazone for diminishing postoperative neutrophil infiltration into ME, wherein the guanylhydrazone is to be administered prior to surgery. The histogram shows neutrophil infiltration into rat ME (neutrophil counts per field) and corresponds to FIG. 5. In particular, CNI-1493 was exchanged for the tetravalent methanesulfonic acid salt (mesylate salt) of the compound shown in FIG. 1 (CPSI-2364) and the route of administration was changed to p.o. Myeloperoxidase staining for detection of neutrophils in rat ME whole mounts was performed 24 h after IM.

FIG. 11: Is a histogram showing the effects of the use of a guanylhydrazone for diminishing postoperative neutrophil infiltration into ME, wherein the guanylhydrazone is to be administered prior to surgery. The histogram shows neutrophil infiltration into mouse ME (neutrophil counts per field). Again, CNI-1493 was exchanged for CPSI-2364 and the route of administration was changed to p.o.

FIG. 12: Shows the effect of the use of a guanylhydrazone for reducing the postoperative nitric oxide (NO) production in ME. It corresponds to FIG. 6 and shows the nitric oxide (NO) production in supernatants of rat ME. Again, CNI-1493 was exchanged for CPSI-2364 and the route of administration was changed to p.o. NO production was determined photometrically by Griess reaction.

FIG. 13: Shows the effect of the use of a guanylhydrazone for the prevention of postoperative contractile impairment of jejunal smooth muscle. It corresponds to FIG. 7 and shows a measurement of jejunal smooth muscle contractility in vitro. Again, CNI-1493 was exchanged for CPSI-2364 and the route of administration was changed to p.o. Spontaneous and bethanechol-induced muscle contractility was recorded.

FIG. 14: Shows the effect of the use of a guanylhydrazone for the prevention of a postoperative delay in GIT and colonic transit. It corresponds to FIG. 8b and shows the in vivo gastrointestinal transit (GIT). Again, CNI-1493 was exchanged for CPSI-2364 and the route of administration was changed to p.o.

FIG. 15: Shows the effect of the use of a guanylhydrazone for the reduction of postoperative mucosal injury in intestinal villi and in crypts as determined by Park scoring. It shows the results of the determination of the intestinal injury in the rat model. Syngeneic orthotopic small intestinal transplantation was performed on animals treated with or without CPSI-2364 (1 mg/kg i.v.) as described below and the intestinal injury was graded by the Park score 3 and 18 hours after reperfusion.

FIG. 16: Shows the effect of the use of a guanylhydrazone for the reduction of postoperative myeloperoxidase positive neutrophil infiltration into the muscularis. It shows the results of the determination of myeloperoxidase positive neutrophil infiltration into the muscularis with or without prior treatment with CPSI-2364 in the rat model. Syngeneic orthotopic small intestinal transplantation was performed on animals treated with or without CPSI-2364 (1 mg/kg i.v.) as described below and the number of infiltrated MPO-positive neutrophil cells was determined 18 hours after reperfusion.

FIG. 17: Shows the effect of the use of a guanylhydrazone for the reduction of postoperative ED1 positive cell infiltration into the muscularis. It shows the results of the determination of ED1 positive cell infiltration into the muscularis with or without prior treatment with CPSI-2364 in the rat model. Syngeneic orthotopic small intestinal transplantation was performed on animals treated with or without CPSI-2364 (1 mg/kg i.v.) as described below and the number of infiltrated ED1 positive cells was determined 18 hours after reperfusion.

FIG. 18: Shows the effect of the use of a guanylhydrazone for reduction of postoperative NO in serum. It shows the results of the determination of nitrite and nitrate in serum. Syngeneic orthotopic small intestinal transplantation was performed on animals treated with or without CPSI-2364 (1 mg/kg i.v.) as described below. Serum of rats with or without prior treatment with CPSI-2364 was examined 3 or 18 hours after reperfusion.

FIG. 19: Shows the effect of the use of a guanylhydrazone for reduction of postoperative IL-6 in serum. It shows the results of the determination of IL-6 in serum. Syngeneic orthotopic small intestinal transplantation was performed on animals treated with or without CPSI-2364 (1 mg/kg i.v.) as described below. Serum of rats with or without prior treatment with CPSI-2364 was examined 3 or 18 hours after reperfusion.

FIG. 20: Shows the effect of the use of a guanylhydrazone for decreasing postoperative contractile impairment of jejunal smooth muscle of a graft. It shows the results of the evaluation of the effect of CPSI-2364 (1 mg/kg, i.v.) administration on the mechanical in vitro activity of the mid-jejunum. Measurements were taken 18 hrs after reperfusion.

FIG. 21: Shows the effect of the use of a guanylhydrazone for decreasing postoperative apoptosis within the smooth muscle layer of the grafts. It shows the results of the effect of CPSI-2364 (1 mg/kg) on apoptosis of the muscularis using the TUNEL-test. Measurements were taken 3 h and 18 h after reperfusion.

FIG. 22: Shows the effect of the use of a guanylhydrazone for increasing postoperative contractility in the jejuna smooth muscle. Male C57BL6/J mice (20 g) underwent intestinal manipulation (IM) or laparotomy without IM (Sham). CPSI-2364 (10 mg/kg) or placebo (mannitol 2.5%) were administered orally (p.o.) or intravenously (i.v.) 90 or 60 minutes before operation, respectively. Twenty-four hours after operation animals were sacrificed. Jejunal circular smooth muscle strips (5-6 per animal) were prepared and contractility was measured under increasing concentrations of bethanecol in an in vitro organ bath setting. n=5-6 animals per group. *p<0.05**p<0.01 vs. IM+placebo p.o by 1-way ANOVA followed by Dunnett's post test.

FIG. 23: Shows the effect of the use of a guanylhydrazone for reducing postoperative myeloperoxidase positive neutrophil infiltration. Male C57BL6/J mice (20 g) underwent intestinal manipulation (IM) or laparotomy without IM (Sham). CPSI-2364 (0.1 or 10 mg/kg) or placebo (0 mg/kg) were administered orally 90 minutes (light gray bars), 6 hours (grey bars) or 16 h (white bars) before operation. Twenty-four hours after operation animals were sacrificed, muscularis. Muscularis whole mount specimen were prepared and stained by Hanker-Yates reagent to detect myeloperoxidase positive neutrophils. Values indicate number of neutrophils per mm² tissue. *p<0.05**p<0.01, ***p<0.001 vs. IM+placebo or indicated probes by 1-way ANOVA followed by Bonferroni's post test (n=3-8 per group).

FIG. 24: Shows the effect of the use of a guanylhydrazone for reducing postoperative bacterial translocation. Male C57BL6/J mice (20 g) underwent intestinal manipulation (IM). Control (CTL) mice were untreated. CPSI-2364 (0.1 or 10 mg/kg) or placebo (2.5% mannitol) were administered orally 90 minutes or 16 h hours before operation by gavage. Twenty-four hours after operation animals were sacrificed and muscularis mesenteric lymph nodes (MLN) were prepared. MLN were weighted, mechanically disrupted and dissociated in 2 ml of 3% thioglycollate medium. 5000 were plated on McConkey agar plates and incubated at 37° C. for 18 hours. Colonies (CFU) were counted and normalized to tissue weight (n=4-8 per group).

FIG. 25: Shows the effect of the use of a guanylhydrazone for reducing postoperative gastrointestinal transit (GIT) time. Male C57BL6/J mice (20 g) underwent intestinal manipulation (IM) or laparotomy without IM (Sham). CPSI-2364 (0.1, 1 or 10 mg/kg) or placebo (2.5% mannitol) were administered orally 90 minutes (light gray bars), 6 hours (grey bars) or 16 h (white bars) before operation. Twenty-four hours after operation animals were fed with 2000 of a FITC dextran solution by gavage. 90 minutes later animals were sacrificed, the complete gastrointestinal tract was removed and divided in 15 parts (Sto=Stomach, Dd, =duodenum, S1-S9=small bowel segments, Cec=Cecum, Coll-3=Colon segments). FITC dextran contents were determined in each segment by fluorometric measurement. Values indicate the geometric centers of FITC dextran distribution. None of the CPSI-2364 treated groups differed significantly from the Sham+Placebo group. **p<0.01, ***p<0.001 vs. IM+placebo or indicated probes by 1-way ANOVA followed by Dunnett's post test (n=3-10 per group).

FIG. 26: Shows the effect of the use of a guanylhydrazone for reducing postoperative colonic transit time. Male C57BL6/J mice (20 g) underwent intestinal manipulation (IM) or laparotomy without IM (Sham). CPSI-2364 (0.1, 1 or 10 mg/kg) or placebo (2.5% mannitol) were administered orally 90 minutes (light gray circles), 6 hours (grey triangles) or 16 h (white squares) before operation. Twenty-four hours after operation a 2 mm glass ball was inserted by a metal rod 3 cm into the colon. Excretion time of the ball was measured in seconds. None of the CPSI-2364 treated group differed significantly from Sham+Placebo group. ***p<0.001 vs. IM+placebo or indicated probes by 1-way ANOVA followed by Bonferroni's post test (n=3-11 per group).

FIG. 27: Shows the effect of the use of a guanylhydrazone for reducing postoperative nitric oxide production in the small bowel. Male C57BL6/J mice (20 g) underwent intestinal manipulation (IM) or laparotomy without IM (Sham). CPSI-2364 (10 mg/kg) or placebo (2.5% mannitol) were administered orally 90 minutes or intravenously 60 minutes before operation. Twenty-four hours after operation small bowel muscularis was prepared and cultured for additional 24 hours in 1 ml DMEM culture medium. Cell-free culture supernatant was analyzed for nitric oxide and its metabolites by Griess reaction. Values were normalized by tissue weight. None of the CPSI-2364 treated group differed significantly different from Sham+Placebo group. **<p0.01 and ***p<0.001 vs. IM+placebo or indicated probes by 1-way ANOVA followed by Bonferroni's post test (n=4-5 per group).

FIG. 28: Shows the effect of the use of a guanylhydrazone for reducing the time to first observed postoperative defacation. Swine underwent intestinal manipulation (IM) or laparotomy without IM (Sham). CPSI-2364 (1 mg/kg) or placebo (mannitol 2.5%) were administered orally (p.o.) twice at 14 hours and 3 hours before operation or intravenously (i.v.) once at 2 hours before operation. Three, 6 and 24 hours after operation animals were examined for first defecation.

FIG. 29: Shows the effect of the use of a guanylhydrazone for increasing the postoperative contractile force in jejuna smooth muscle. Swine underwent intestinal manipulation (IM) or laparotomy without IM (Sham). CPSI-2364 (1 mg/kg) or placebo (mannitol 2.5%) were administered orally (p.o.) twice at 14 hours and 3 hours before operation or intravenously (i.v.) once at 2 hours before operation. Twenty-four hours after operation animals were sacrificed. Jejunal circular smooth muscle strips were prepared and contractility was measured under increasing concentrations of bethanecol in an in vitro organ bath setting. n=5-6 animals per group. *p<0.05**p<0.01 vs. IM+Placebo p.o by 1-way ANOVA followed by Bonferroni's post test.

FIG. 30: Shows the effect of the use of a guanylhydrazone on postoperative intestinal anastomotic healing. Swine were fed with 1 mg/kg CPSI-2364 or placebo (2.5% mannitol) 14 hours and 3 hours before operation. In the iv group, 1 mg/kg CPSI 2364 was fed once 2 hours before operation. After premedication animals were intubated and a central venous catheter was applied. A laparotomy was performed under sterile conditions. The distal part of the colon, comparable to the human sigmoid, was identified and cut through followed by an anastomosis with continuous suture (4/0 monocryl).On postoperative day 6 (pod6) animals were sacrificed, the anastomotic region was removed (Ana) and analyzed for hydroxyproline content and its strength by determination of the bursting pressure (in mmHg). No differences were observed in the bursting pressure levels (A) and hydroxyproline content (B), indicating that preoperative oral CPSI-2364 treatment does not affect intestinal anastomotic healing.

FIG. 31: Shows the effect of the use of a guanylhydrazone for reducing postoperative myeloperoxidase activity and neutrophil infiltration. Swine underwent intestinal manipulation (IM) or laparotomy without IM (Sham). CPSI-2364 (1 mg/kg) or placebo (mannitol 2.5%) were administered orally (p.o.) twice at 14 hours and 3 hours before operation or intravenously (i.v.) once at 2 hours before operation. Twenty-four hours after operation animals were sacrificed. Muscularis specimens were prepared and myeloperoxidase activity was measured to determine the degree of neutrophil infiltration. **p<0.01 vs. indicated probes by 1-way ANOVA followed by Bonferroni's post test (n=6 per group).

FIG. 32: Shows the effect of the use of a guanylhydrazone for inhibiting the macrophage chemoattractant protein −1. Swine underwent intestinal manipulation (IM) or laparotomy without IM (Sham). CPSI-2364 (1 mg/kg) or placebo (mannitol 2.5%) were administered orally (p.o.) twice at 14 hours and 3 hours before operation or intravenously (i.v.) once at 2 hours before operation. Twenty-four hours after operation animals were sacrificed. Muscularis specimens were analyzed for macrophage chemoattractant protein −1 (MCP-1) mRNA expression by quantitative real time PCR. Expression levels were normalized to muscularis of untreated swine (control). ***p<0.001 vs. indicated probes by 1-way ANOVA followed by Bonferroni's post test.

FIG. 33: Shows the effect of the use of a guanylhydrazone for reducing the gastrointestinal transit (GIT) time. Swine underwent intestinal manipulation (IM) or laparotomy without IM (Sham). CPSI-2364 (1 mg/kg) or placebo (mannitol 2.5%) were administered orally (p.o.) twice at 14 hours and 3 hours before operation or intravenously (i.v.) once at 2 hours before operation. At the end of the operation, 15 radio-opaque globes were placed in the proximal small bowel. Twenty-four hours after operation animals were sacrificed and the radio-opaque globes were detected by x-rays radiography and the distribution along the gastrointestinal tract was calculated as the geometric center **p<0.001 vs. indicated probes by 1-way ANOVA followed by Bonferroni's post test. GC for the individual as mean+/−standard error of the mean were as follows: Sham+Placebo: 11.60+/−0.49; IM+Placebo: 7.67+/−1.74; IM+CPSI-2364 p.o.: 13.00+/−0.27; IM+CPSI-2364 i.v. 14.01+/−0.44.

### DETAILED DESCRIPTION OF THE SEVERAL EMBODIMENTS

One embodiment is based on the surprising finding that surgical manipulation (resulting in a mechanical trauma) initiates a severe inflammation in the muscularis external (ME) that is mediated by early activation of the p38-MAPK pathway. Unexpectedly, it has now been found that guanylhydrazones inhibit this activation, diminish the subsequent inflammation and prevent suppression of smooth muscle function and gastrointestinal motility if administered prior to surgery. Furthermore, the ischemia reperfusion injury occurring during small bowel transplantation induces severe inflammation in graft muscularis leading to graft dysmotility, and this, too, may be inhibited by the preoperative administration of guanylhydrazones, which is surprising. For the first time, it has unexpectedly been found that this iatrogenic complication may be ameliorated by a prophylactic treatment with guanylhydrazones.

In one embodiment, the guanylhydrazones are macrophage-specific inhibitors of p38 MAPK (mitogen activated protein kinase) phosphorylation and/or abrogate nitric oxide production within the tunica muscularis. In other embodiments, the guanylhydrazones are CNI-1493 or CPSI-2364.

One embodiment is directed to the pharmaceutical use of at least one guanylhydrazone and/or salt thereof to prevent postoperative inflammatory responses of the intestine. Another embodiment is directed to the pharmaceutical use of at least one guanylhydrazone and/or salt thereof to prevent iatrogenic complications associated with postoperative inflammatory response of the intestine. Another embodiment is directed to the pharmaceutical use of at least one guanylhydrazone and/or salt thereof to prevent postoperative ileus. Another embodiment is directed to the pharmaceutical use of at least one guanylhydrazone and/or salt thereof to prevent ischemia reperfusion injury. Another embodiment is directed to the pharmaceutical use of at least one guanylhydrazone and/or salt thereof to prevent postoperative inflammatory responses of the intestine and/or iatrogenic complications associated therewith, while not completely suppressing the inflammatory reaction that is crucial for healing processes of the body.

One embodiment is directed to the pharmaceutical use of at least one guanylhydrazone and/or salt thereof to ameliorate postoperative inflammatory responses of the intestine. Another embodiment is directed to the pharmaceutical use of at least one guanylhydrazone and/or salt thereof to ameliorate iatrogenic complications associated with postoperative inflammatory response of the intestine. Another embodiment is directed to the pharmaceutical use of at least one guanylhydrazone and/or salt thereof to ameliorate postoperative ileus. Another embodiment is directed to the pharmaceutical use of at least one guanylhydrazone and/or salt thereof to ameliorate ischemia reperfusion injury. Another embodiment is directed to the pharmaceutical use of at least one guanylhydrazone and/or salt thereof to ameliorate postoperative inflammatory responses of the intestine and/or iatrogenic complications associated therewith, while not completely suppressing the inflammatory reaction that is crucial for healing processes of the body.

Unexpectedly, it has now been found that guanylhydrazones and/or salts thereof, if administered prior to surgery, are capable of preventing a postoperative inflammatory reaction of the intestine in response to a mechanical trauma as, for example, caused by surgical procedures. As demonstrated herein for the first time, prophylactic administration of a guanylhydrazone and/or salt thereof significantly decreases proinflammatory gene expression and inflammation in test animals following IM. Although inflammation was still severe in these test animals, unexpectedly, smooth muscle dysfunction and gastrointestinal transit were completely normalized in test animals that received preoperative guanylhydrazone as compared to control animals that did not receive guanyhydrazone.

The initiation of a complex and severe inflammation within the gut tunica muscularis results in smooth muscle dysfunction and subsequently gastrointestinal dysmotility (Kalff J C, Schraut W H, Simmons R L et al. Surgical manipulation of the gut elicits an intestinal muscularis inflammatory response resulting in postsurgical ileus. Ann Surg 1998; 228:652-663; Kalff J C, Carlos T M, Schraut W H et al. Surgically induced leukocytic infiltrates within the rat intestinal muscularis mediate postoperative ileus. Gastroenterology 1999; 117:378-387; Schwarz N T, Kalff J C, Turler A et al. Prostanoid Production Via COX-2 as a Causative Mechanism of Rodent Postoperative Ileus. Gastroenterology 2001; 121:1354-1371; Turler A, Moore B A, Pezzone M A et al. Colonic postoperative inflammatory ileus in the rat. Ann Surg 2002; 236:56-66; Turler A, Schnurr C, Nakao A et al. Endogenous Endotoxin Participates in Causing a Panenteric Inflammatory Ileus After Colonic Surgery. Ann Surg 2007; 245:734-744). Thus, postoperative intestinal inflammation of the intestine may cause postoperative ileus (POI).

The underlying inflammatory cascade includes the activation of resident muscularis macrophages and subsequently the extravasation of immunocompetent leukocytes (Kalff J C, Schraut W H, Simmons R L et al. Surgical manipulation of the gut elicits an intestinal muscularis inflammatory response resulting in postsurgical ileus. Ann Surg 1998; 228:652-663; Kalff J C, Carlos T M, Schraut W H et al. Surgically induced leukocytic infiltrates within the rat intestinal muscularis mediate postoperative ileus. Gastroenterology 1999; 117:378-387).

Both activated cell populations liberate substances, such as nitric oxide (NO) and prostaglandins, which directly mediate postoperative smooth muscle dysfunction (Kalff J C, Schraut W H, Billiar T R et al. Role of inducible nitric oxide synthase in postoperative intestinal smooth muscle dysfunction in rodents. Gastroenterology 2000; 118:316-327; Schwarz N T, Kalff J C, Turler A et al. Prostanoid Production Via COX-2 as a Causative Mechanism of Rodent Postoperative Ileus. Gastroenterology 2001; 121:1354-1371).

Likewise, ischemia reperfusion injuries after transplantation surgery are additional iatrogenic complications associated with a severe inflammatory response. The absence of oxygen and nutrients from blood creates a condition in which the restoration of circulation results in inflammation and oxidative damage through the induction of oxidative stress rather than restoration of normal function. The ischemia reperfusion injury occurring during small bowel transplantation induces severe inflammation in graft muscularis leading to graft dysmotility consequently increasing the risk of bacterial translocation and infectious complications.

Although knowledge of the inflammatory cascade and the affected cell populations and the physiological effects underlying these severe postoperative inflammatory responses has increased widely in the last few years, there remains a lack of knowledge about proinflammatory signaling pathways. It has now been found that the proinflammatory p38-MAPK pathway is involved in these postoperative inflammatory responses. It has also now been found that interrupting this pathway by a guanylhydrazone or salt thereof which is administered prior to an established postoperative inflammatory reaction is a more selective and promising strategy to prevent intestinal inflammation and prevent POI and also prevent ischemia reperfusion injuries of grafts, and particularly of grafts in small bowel transplantation (SBTx). Thus, the inactivation of (resident muscularis) macrophages by a prophylactic administration of the guanylhydrazones results in a diminished intestinal inflammation and thus prevents gastrointestinal dysmotility and ischemia reperfusion injuries of grafts in small bowel transplantation.

One embodiment relates to the administration of one or more guanylhydrazone compounds and/or salts thereof to a subject at risk of or suffering from any of the following: surgical or other manipulation of the intestine, postoperative ileus, inflammation in the muscularis externa (ME) that is mediated by early activation of the p38-MAPK pathway, inflammation after surgical or other manipulation of the intestine, suppression of smooth muscle function, suppression of gastrointestinal motility, ischemia reperfusion injury, ischemia reperfusion injury occurring during small bowel transplantation, inflammation in graft muscularis, graft dysmotility, iatrogenic complications, p38 MAPK (mitogen activated protein kinase) phosphorylation and/or nitric oxide production within the mucosa, submucosa, and/or tunica muscularis, postoperative inflammatory response of the intestine, iatrogenic complications associated with postoperative inflammatory response of the intestine, postoperative inflammatory reaction of the intestine in response to mechanical trauma, increased proinflammatory gene expression and inflammation following intestinal manipulation, increased or delayed gastrointestinal transit, increased or delayed colonic transit, increased or delayed time to first defecation following surgical or other manipulation of the intestine, any combination thereof, and the like.

Another embodiment relates to the administration of one or more guanylhydrazone compounds and/or salts thereof to a subject to prevent, ameliorate the effects of, or reduce the suffering from any of the following: surgical or other manipulation of the intestine, postoperative ileus, inflammation in the muscularis externa (ME) that is mediated by early activation of the p38-MAPK pathway, inflammation after surgical or other manipulation of the intestine, suppression of smooth muscle function, suppression of gastrointestinal motility, ischemia reperfusion injury, ischemia reperfusion injury occurring during small bowel transplantation, inflammation in graft muscularis, graft dysmotility, iatrogenic complications, p38 MAPK (mitogen activated protein kinase) phosphorylation and/or nitric oxide production within the mucosa, submucosa, and/or tunica muscularis, postoperative inflammatory response of the intestine, iatrogenic complications associated with postoperative inflammatory response of the intestine, postoperative inflammatory reaction of the intestine in response to mechanical trauma, increased proinflammatory gene expression and inflammation following intestinal manipulation, increased or delayed gastrointestinal transit, increased or delayed colonic transit, increased or delayed time to first defecation following surgical or other manipulation of the intestine, any combination thereof, and the like.

Another embodiment relates to the administration of one or more guanylhydrazone compounds and/or salts thereof to a subject prior to any of the following: surgical or other manipulation of the intestine, postoperative ileus, inflammation in the muscularis externa (ME) that is mediated by early activation of the p38-MAPK pathway, inflammation after surgical or other manipulation of the intestine, suppression of smooth muscle function, suppression of gastrointestinal motility, ischemia reperfusion injury, ischemia reperfusion injury occurring during small bowel transplantation, inflammation in graft muscularis, graft dysmotility, iatrogenic complications, p38 MAPK (mitogen activated protein kinase) phosphorylation and/or nitric oxide production within the mucosa, submucosa, and/or tunica muscularis, postoperative inflammatory response of the intestine, iatrogenic complications associated with postoperative inflammatory response of the intestine, postoperative inflammatory reaction of the intestine in response to mechanical trauma, increased proinflammatory gene expression and inflammation following intestinal manipulation, increased or delayed gastrointestinal transit, increased or delayed colonic transit, increased or delayed time to first defecation following surgical or other manipulation of the intestine, any combination thereof, and the like.

One embodiment relates to the administration of a pharmaceutical composition comprising one or more guanylhydrazone compounds and/or salts thereof to a subject at risk of or suffering from any of the following: surgical or other manipulation of the intestine, postoperative ileus, inflammation in the muscularis externa (ME) that is mediated by early activation of the p38-MAPK pathway, inflammation after surgical or other manipulation of the intestine, suppression of smooth muscle function, suppression of gastrointestinal motility, ischemia reperfusion injury, ischemia reperfusion injury occurring during small bowel transplantation, inflammation in graft muscularis, graft dysmotility, iatrogenic complications, p38 MAPK (mitogen activated protein kinase) phosphorylation and/or nitric oxide production within the mucosa, submucosa, and/or tunica muscularis, postoperative inflammatory response of the intestine, iatrogenic complications associated with postoperative inflammatory response of the intestine, postoperative inflammatory reaction of the intestine in response to mechanical trauma, increased proinflammatory gene expression and inflammation following intestinal manipulation, increased or delayed gastrointestinal transit, increased or delayed colonic transit, increased or delayed time to first defecation following surgical or other manipulation of the intestine, any combination thereof, and the like.

Another embodiment relates to the administration of a pharmaceutical composition comprising one or more guanylhydrazone compounds and/or salts thereof to a subject to prevent, ameliorate the effects of, or reduce the suffering from any of the following: surgical or other manipulation of the intestine, postoperative ileus, inflammation in the muscularis externa (ME) that is mediated by early activation of the p38-MAPK pathway, inflammation after surgical or other manipulation of the intestine, suppression of smooth muscle function, suppression of gastrointestinal motility, ischemia reperfusion injury, ischemia reperfusion injury occurring during small bowel transplantation, inflammation in graft muscularis, graft dysmotility, iatrogenic complications, p38 MAPK (mitogen activated protein kinase) phosphorylation and/or nitric oxide production within the mucosa, submucosa, and/or tunica muscularis, postoperative inflammatory response of the intestine, iatrogenic complications associated with postoperative inflammatory response of the intestine, postoperative inflammatory reaction of the intestine in response to mechanical trauma, increased proinflammatory gene expression and inflammation following intestinal manipulation, increased or delayed gastrointestinal transit, increased or delayed colonic transit, increased or delayed time to first defecation following surgical or other manipulation of the intestine, any combination thereof, and the like.

Another embodiment relates to the administration of a pharmaceutical composition comprising one or more guanylhydrazone compounds and/or salts thereof to a subject prior to any of the following: surgical or other manipulation of the intestine, postoperative ileus, inflammation in the muscularis externa (ME) that is mediated by early activation of the p38-MAPK pathway, inflammation after surgical or other manipulation of the intestine, suppression of smooth muscle function, suppression of gastrointestinal motility, ischemia reperfusion injury, ischemia reperfusion injury occurring during small bowel transplantation, inflammation in graft muscularis, graft dysmotility, iatrogenic complications, p38 MAPK (mitogen activated protein kinase) phosphorylation and/or nitric oxide production within the mucosa, submucosa, and/or tunica muscularis, postoperative inflammatory response of the intestine, iatrogenic complications associated with postoperative inflammatory response of the intestine, postoperative inflammatory reaction of the intestine in response to mechanical trauma, increased proinflammatory gene expression and inflammation following intestinal manipulation, increased or delayed gastrointestinal transit, increased or delayed colonic transit, increased or delayed time to first defecation following surgical or other manipulation of the intestine, any combination thereof, and the like.

In one embodiment, the administration of one or more guanylhydrazone compounds and/or salts thereof to a subject prevents, ameliorates the effects of, or reduces the suffering from any of the following: surgical or other manipulation of the intestine, postoperative ileus, inflammation in the muscularis externa (ME) that is mediated by early activation of the p38-MAPK pathway, inflammation after surgical or other manipulation of the intestine, suppression of smooth muscle function, suppression of gastrointestinal motility, ischemia reperfusion injury, ischemia reperfusion injury occurring during small bowel transplantation, inflammation in graft muscularis, graft dysmotility, iatrogenic complications, p38 MAPK (mitogen activated protein kinase) phosphorylation and/or nitric oxide production within the mucosa, submucosa, and/or tunica muscularis, postoperative inflammatory response of the intestine, iatrogenic complications associated with postoperative inflammatory response of the intestine, postoperative inflammatory reaction of the intestine in response to mechanical trauma, increased proinflammatory gene expression and inflammation following intestinal manipulation, increased or delayed gastrointestinal transit, increased or delayed colonic transit, increased or delayed time to first defecation following surgical or other manipulation of the intestine, any combination thereof, and the like, but the administration does not completely suppress an inflammatory reaction that is crucial for the healing processes of the body.

In one embodiment, administration of the guanylhydrazone and/or salt thereof does not adversely affect anastomotic wound healing.

In one embodiment, the ileus, inflammation in the muscularis externa (ME) that is mediated by early activation of the p38-MAPK pathway, inflammation of the intestine, suppression of smooth muscle function, suppression of gastrointestinal motility, ischemia reperfusion injury, inflammation in graft muscularis, graft dysmotility, iatrogenic complications, p38 MAPK (mitogen activated protein kinase) phosphorylation and/or nitric oxide production within the mucosa, submucosa, and/or tunica muscularis, inflammatory response of the intestine, iatrogenic complications associated with inflammatory response of the intestine, postoperative inflammatory reaction of the intestine, increased proinflammatory gene expression and inflammation, increased or delayed gastrointestinal transit, increased or delayed colonic transit, increased or delayed time to first defecation, or any combination thereof, and the like, arise as a function of a surgical operation, intestinal manipulation, or other mechanical trauma.

The guanylhydrazones suitable for use herein are not particularly limited. Non-limiting examples include those guanylhydrazones disclosed in U.S. Pat. Nos. 7,244,765, 7,291,647, and/or 5,599,984, which are hereby incorporated by reference in their entirety, although others may be used. For example, guanylhydrazones disclosed at column 1, line 56 through column 10, line 37 of U.S. Pat. No. 7,291,647 and column 2, line 65 through column 14, line 37 of U.S. Pat. No. 7,244,765 may be used.

In one embodiment, a guanylhydrazone is any compound having one or more guanylhydrazone group of the formula: NH₂C(═NH)—NH—N═.

For example, the guanylhydrazones or salts thereof may suitably have the formula:

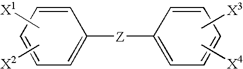

wherein X¹, X², X³, and X⁴ each independently represent H, GhyCH—, GhyCCH₃—, or CH₃CO—, with the provisos that X¹, X², X³ and X⁴ are not simultaneously H;

wherein Z is one or more selected from the group consisting of:

-(A¹)ₐ-(CR²R³)ₓ-(A²)_{b}-;
-(A¹)ₐ-(CR²R³)ₓ-Qₘ-(CR⁴R⁵)_{y}-(A²)_{b}; and
-(A¹)ₐ-(CR²R³)ₓ-Qₘ-(CR⁴R⁵)_{y}-Tₙ-(CR⁶R⁷)_{z}-(A²)_{b}-;
and combinations thereof;

wherein a is selected from the group consisting of 0, 1, 2, 3, 4, 5, 6, 7, 8, and 9;

wherein b is selected from the group consisting of 0, 1, 2, 3, 4, 5, 6, 7, 8, and 9;

wherein x is selected from the group consisting of 0, 1, 2, 3, 4, 5, 6, 7, 8, and 9;

wherein y is selected from the group consisting of 0, 1, 2, 3, 4, 5, 6, 7, 8, and 9;

wherein z is selected from the group consisting of 0, 1, 2, 3, 4, 5, 6, 7, 8, and 9;

wherein m is selected from the group consisting of 0, 1, 2, 3, 4, 5, 6, 7, 8, and 9;

wherein n is selected from the group consisting of 0, 1, 2, 3, 4, 5, 6, 7, 8, and 9;

wherein A¹ and A² are each independently selected from the group consisting of —NR⁸(CO)NR⁹—, —(CO)NR⁸—, —NR⁸(CO)—, —NR⁸—, —O—, —S—, —S(═O)—, —SO₂—, —SO₂NR⁸—, —NR⁸SO₂—, and salts thereof;

wherein Q and T are each independently selected from the group consisting of NR¹⁰(CO)NR¹¹—, —(CO)NR¹⁰—, —NR¹⁰(CO)—, —NR¹⁰—, —O—, —S—, —S(═O)—, —SO₂—, —SO₂NR¹⁰—, —NR¹⁰SO₂—, salts thereof, branched or unbranched, saturated or unsaturated, substituted or unsubstituted C₁-C₂₀ alkylene, saturated or unsaturated, substituted or unsubstituted C₃-C₂₀ cycloalkylene, substituted or unsubstituted C₅-C₂₅ arylene, and combinations thereof;

wherein one or more carbon atoms in any of said alkylene, cycloalkylene or arylene in said Q and/or T may each be independently replaced with one or more heteroatoms selected from the group consisting of nitrogen, oxygen, sulfur, and a combination thereof;

and wherein when substituted, said alkylene, cycloalkylene or arylene in said Q and/or T are each independently substituted with one or more substituent groups selected from the group consisting of hydroxy, halo, bromo, chloro, iodo, fluoro, —N₃, —CN—, —NC, —SH, —NO₂, —NH₂, (C₁-C₂₀)alkyl, phenyl, (C₃-C₂₀)cycloalkyl, (C₁-C₂₀)alkoxy, (C₃-C₂₅)heteroaryl, (C₃-C₂₅)heterocyclic, (C₂-C₂₀)alkenyl, (C₃-C₂₀) cycloalkenyl, (C₂-C₂₀)alkynyl, (C₅-C₂₀)cycloalkynyl, (C₅-C₂₅)aryl, perhalo(C₁-C₂₀)alkyl, (C₁-C₂₀)alkyl-O—, phenyl-O—, (C₃-C₂₀)cycloalkyl-O—, (C₃-C₂₅)heteroaryl-O—, (C₃-C₂₅)heterocyclic-O—, (C₂-C₂₀)alkenyl-O—, (C₃-C₂₀) cycloalkenyl-O—, (C₂-C₂₀)alkynyl-O—, (C₅-C₂₀)cycloalkynyl-O—, (C₅-C₂₅)aryl-O—, perhalo(C₁-C₂₀)alkyl-O—, (C₁-C₂₀)alkyl-S—, phenyl-S—, (C₃-C₂₀)cycloalkyl-S—, (C₃-C₂₅)heteroaryl-S—, (C₃-C₂₅)heterocyclic-S—, (C₂-C₂₀)alkenyl-S—, (C₃-C₂₀)cycloalkenyl-S—, (C₂-C₂₀)alkynyl-S—, (C₅-C₂₀)cycloalkynyl-S—, (C₅-C₂₅)aryl-S—, perhalo(C₁-C₂₀)alkyl-S—, (C₁-C₂₀)alkyl-SO₂—, phenyl-SO₂—, (C₃-C₂₀)cycloalkyl-SO₂—, (C₁-C₂₀)alkoxy-SO₂—, (C₃-C₂₅)heteroaryl-SO₂—, (C₃-C₂₅)heterocyclic-SO₂—, (C₂-C₂₀)alkenyl-SO₂—, (C₃-C₂₀)cycloalkenyl-SO₂—, (C₂-C₂₀)alkynyl-SO₂—, (C₅-C₂₀)cycloalkynyl-SO₂—, (C₅-C₂₅)aryl-SO₂—, perhalo(C₁-C₂₀)alkyl-SO₂—, H₂N—SO₂—, (C₁-C₂₀)alkyl-NH—SO₂—, phenyl-NH—SO₂—, (C₃-C₂₀)cycloalkyl-NH—SO₂—, (C₁-C₂₀)alkoxy-NH—SO₂—, (C₃-C₂₅)heteroaryl-NH—SO₂—, (C₃-C₂₅)heterocyclic-NH—SO₂—, (C₂-C₂₀)alkenyl-NH—SO₂—, (C₃-C₂₀)cycloalkenyl-NH—SO₂—, (C₂-C₂₀)alkynyl-NH—SO₂—, (C₅-C₂₀)cycloalkynyl-NH—SO₂—, (C₅-C₂₅)aryl-NH—SO₂—, perhalo(C₁-C₂₀)alkyl-NH—SO₂—, {(C₁-C₂₀)alkyl}₂N—SO₂—, {phenyl}₂N—SO₂—, {(C₃-C₂₀)cycloalkyl}₂N—SO₂—, {(C₁-C₂₀)alkoxyl}₂N—SO₂—, {(C₃-C₂₅)heteroaryl}₂N—SO₂—, {(C₃-C₂₅)heterocyclic}₂N—SO₂—, {(C₂-C₂₀)alkenyl}₂N—SO₂—, {(C₂-C₂₀)alkynyl}₂N—SO₂—, {(C₅-C₂₀)cycloalkynyl}₂N—SO₂—, {(C₅-C₂₅)aryl}₂N—SO₂—, {perhalo(C₁-C₂₀)alkyl}₂N—SO₂—, (C₁-C₂₀)alkyl-SO₂—NH—, phenyl-SO₂—NH—, (C₃-C₂₀)cycloalkyl-SO₂—NH—, (C₁-C₂₀)alkoxy-SO₂—NH—, (C₃-C₂₅)heteroaryl-SO₂—NH—, (C₃-C₂₅)heterocyclic-SO₂—NH—, (C₂-C₂₀)alkenyl-SO₂—NH—, (C₃-C₂₀)cycloalkenyl-SO₂—NH—, (C₂-C₂₀)alkynyl-SO₂—NH—, (C₅-C₂₀)cycloalkynyl-SO₂—NH—, (C₅-C₂₅)aryl-SO₂—NH—, perhalo(C₁-C₂₀)alkyl-SO₂—NH—, (C₁-C₂₀)alkyl-NH—, phenyl-NH—, (C₃-C₂₀)cycloalkyl-NH—, (C₁-C₂₀)alkoxy-NH—, (C₃-C₂₅)heteroaryl-NH—, (C₃-C₂₅)heterocyclic-NH—, (C₂-C₂₀)alkenyl-NH—, (C₃-C₂₀)cycloalkenyl-NH—, (C₂-C₂₀)alkynyl-NH—, (C₅-C₂₀)cycloalkynyl-NH—, (C₅-C₂₅)aryl-NH—, perhalo(C₁-C₂₀)alkyl-NH—, {(C₁-C₂₀)alkyl}₂N—, {phenyl}₂N—, {(C₃-C₂₀)cycloalkyl}₂N—, {(C₁-C₂₀)alkoxy}₂N—, {(C₃-C₂₅)heteroaryl}₂N—, {(C₃-C₂₅)heterocyclic}₂N—, {(C₂-C₂₀)alkenyl}₂N—, {(C₃-C₂₀)cycloalkenyl}₂N—, {(C₂-C₂₀)alkynyl}₂N—, {(C₅-C₂₀)cycloalkynyl}₂N—, {(C₅-C₂₅)aryl}₂N—, {perhalo(C₁-C₂₀)alkyl}₂N—, (C₁-C₂₀)alkyl-(C═O)—NH—, phenyl-(C═O)—NH—, (C₃-C₂₀)cycloalkyl-(C═O)—NH—, (C₁-C₂₀)alkoxy-(C═O)—NH—, (C₃-C₂₅)heteroaryl-(C═O)—NH—, (C₃-C₂₅)heterocyclic-(C═O)—NH—, (C₂-C₂₀)alkenyl-(C═O)—NH—, (C₃-C₂₀)cycloalkenyl-(C═O)—NH—, (C₂-C₂₀)alkynyl-(C═O)—NH—, (C₅-C₂₀)cycloalkynyl-(C═O)—NH—, (C₅-C₂₅)aryl-(C═O)—NH—, perhalo(C₁-C₂₀)alkyl-(C═O)—NH—, (C₁-C₂₀)alkyl-(C═O)—{((C₁-C₂₀)alkyl)N}—, phenyl-(C═O)—{((C₁-C₂₀)alkyl)N}—, (C₃-C₂₀)cycloalkyl-(C═O)-{((C₁-C₂₀)alkyl)N}—, (C₁-C₂₀)alkoxy-(C═O)—{((C₁-C₂₀)alkyl)N}—, (C₃-C₂₅)heteroaryl-(C═O)—{((C₁-C₂₀)alkyl)N}—, (C₃-C₂₅)heterocyclic-(C═O)—{((C₁-C₂₀)alkyl)N}—, (C₂-C₂₀)alkenyl-(C═O)—{((C₁-C₂₀)alkyl)N}—, (C₃-C₂₀)cycloalkenyl-(C═O)—{((C₁-C₂₀)alkyl)N}—, (C₂-C₂₀)alkynyl-(C═O)—{((C₁-C₂₀)alkyl)N}—, (C₅-C₂₀)cycloalkynyl-(C═O)—{((C₁-C₂₀)alkyl)N}—, (C₅-C₂₅)aryl-(C═O)—{((C₁-C₂₀)alkyl)N}—, perhalo(C₁-C₂₀)alkyl-(C═O)—{((C₁-C₂₀)alkyl)N}—, phenyl-(C═O)—NH—, phenyl-(C═O)-{(phenyl)N}—, (C₁-C₂₀)alkyl-(C═O)-{(phenyl)N}—, (C₃-C₂₀)cycloalkyl-(C═O)-{(phenyl)N}—, (C₁-C₂₀)alkoxy-(C═O)-{(phenyl)N}—, (C₃-C₂₅)heteroaryl-(C═O)-{(phenyl)N}—, (C₃-C₂₅)heterocyclic-(C═O)-{(phenyl)N}—, (C₂-C₂₀)alkenyl-(C═O)-{(phenyl)N}—, (C₃-C₂₀)cycloalkenyl-(C═O)-{(phenyl)N}—, (C₂-C₂₀)alkynyl-(C═O)-{(phenyl)N}—, (C₅-C₂₀)cycloalkynyl-(C═O)-{(phenyl)N}—, (C₅-C₂₅)aryl-(C═O)-{(phenyl)N}—, perhalo(C₁-C₂₀)alkyl-(C═O)-{(phenyl)N}—, H₂N(C═O)—, (C₁-C₂₀)alkyl-NH—(C═O)—, phenyl-NH—(C═O)—, (C₃-C₂₀)cycloalkyl-NH—(C═O)—, (C₁-C₂₀)alkoxy-NH—(C═O)—, (C₃-C₂₅)heteroaryl-NH—(C═O)—, (C₃-C₂₅)heterocyclic-NH—(C═O)—, (C₂-C₂₀)alkenyl-NH—(C═O)—, (C₃-C₂₀) cycloalkenyl-NH—(C═O)—, (C₂-C₂₀)alkynyl-NH—(C═O)—, (C₅-C₂₀)cycloalkynyl-NH—(C═O)—, (C₅-C₂₅)aryl-NH—(C═O)—, perhalo(C₁-C₂₀)alkyl-NH—(C═O)—, {C₁-C₂₀)alkyl}₂N—(C═O)—, {phenyl}{(C₁-C₂₀)alkyl}N—(C═O)—, {(C₃-C₂₀)cycloalkyl}{(C₁-C₂₀)alkyl}N—(C═O)—, {(C₁-C₂₀)alkoxy}{(C₁-C₂₀)alkyl}N—(C═O)—, {(C₃-C₂₅)heteroaryl}{(C₁-C₂₀)alkyl}N—(C═O)—, {(C₃-C₂₅)heterocyolic}{(C₁-C₂₀)alkyl}N—(C═O)—, {(C₂-C₂₀)alkenyl}{(C₁-C₂₀)alkyl}N—(C═O)—, {(C₃-C₂₀)cycloalkenyl}{(C₁-C₂₀)alkyl}N—(C═O)—, {(C₂-C₂₀)alkynyl}{(C₁-C₂₀)alkyl}N—(C═O)—, {(C₅-C₂₀)cycloalkynyl}{(C₁-C₂₀)alkyl}N—(C═O)—, {(C₅-C₂₅)aryl}{(C₁-C₂₀)alkyl}N—(C═O)—, {perhalo(C₁-C₂₀)alkyl}{(C₁-C₂₀)alkyl}N—(C═O)—, {phenyl}₂N—(C═O)—, {(C₃-C₂₀)cycloalkyl}{phenyl}N—(C═O)—, {(C₁-C₂₀)alkoxy}{phenyl}N—(C═O)—, {(C₃-C₂₅)heteroaryl}{phenyl}N—(C═O)—, {(C₃-C₂₅)heterocyclic}{phenyl}N—(C═O)—, {(C₂-C₂₀)alkenyl}{phenyl}N—(C═O)—, {(C₃-C₂₀)cycloalkenyl}{phenyl}N—(C═O)—, {(C₂-C₂₀)alkynyl}{phenyl}N—(C═O)—, {(C₅-C₂₀)cycloalkynyl}{phenyl}N—(C═O)—, {(C₅-C₂₅)aryl}{phenyl}N—(C═O)—, {perhalo(C₁-C₂₀)alkyl}{phenyl}N—(C═O)—, HO—(C═O)—, (C₁-C₂₀)alkyl-(C═O)—, (C₃-C₂₅)heteroaryl-(C═O)—, (C₃-C₂₅)heterocyclic-(C═O)—, (C₂-C₂₀)alkenyl-(C═O)—, (C₃-C₂₀)cycloalkenyl-(C═O)—, (C₂-C₂₀)alkynyl-(C═O)—, (C₅-C₂₅)aryl-(C═O)—, perhalo(C₁-C₂₀)alkyl-(C═O)—, phenyl-(C═O)—, (C₁-C₂₀)alkyl-O—(C═O)—, (C₃-C₂₅)heteroaryl-O—(C═O)—, (C₃-C₂₅)heterocyclic-O—(C═O)—, (C₂-C₂₀)alkenyl-O—(C═O)—, (C₃-C₂₀) cycloalkenyl-O—(C═O)—, (C₂-C₂₀)alkynyl-O—(C═O)—, (C₅-C₂₅)aryl-O—(C═O)—, perhalo(C₁-C₂₀)alkyl-O—(C═O)—, phenyl-O—(C═O)—, (C₁-C₂₀)alkyl-(C═O)—O—, (C₃-C₂₅)heteroaryl-(C═O)—O—, (C₃-C₂₅)heterocyclic-(C═O)—O—, (C₂-C₂₀)alkenyl-(C═O)—O—, (C₃-C₂₀) cycloalkenyl-(C═O)—O—, (C₂-C₂₀)alkynyl-(C═O)—O—, (C₅-C₂₅)aryl-(C═O)—O—, phenyl-(C═O)—O—, perhalo(C₁-C₂₀)alkyl-(C═O)—O—, and salts thereof;

wherein each of the aforesaid (C₁-C₂₀)alkyl, phenyl, (C₃-C₂₀)cycloalkyl, (C₁-C₂₀)alkoxy, (C₃-C₂₅)heteroaryl, (C₃-C₂₅)heterocyclic, (C₂-C₂₀)alkenyl, (C₃-C₂₀)cycloalkenyl, (C₂-C₂₀)alkynyl, (C₅-C₂₀)cycloalkynyl, and (C₅-C₂₅)aryl groups (as substituents on said alkylene, cycloalkylene or arylene of said Q and T) may be optionally and independently substituted by one to four moieties selected from the group consisting of hydroxy, halo, bromo, chloro, iodo, fluoro, —N₃, —CN, —NC, —SH, —NO₂, —NH₂, (C₁-C₂₀)alkyl, phenyl, (C₃-C₂₀)cycloalkyl, (C₁-C₂₀)alkoxy, (C₃-C₂₅)heteroaryl, (C₃-C₂₅)heterocyclic, (C₂-C₂₀)alkenyl, (C₃-C₂₀)cycloalkenyl, (C₂-C₂₀)alkynyl, (C₅-C₂₀)cycloalkynyl, (C₅-C₂₅)aryl, perhalo(C₁-C₂₀)alkyl, (C₁-C₂₀)alkyl-O—, phenyl-O—, (C₃-C₂₀)cycloalkyl-O—, (C₃-C₂₅)heteroaryl-O—, (C₃-C₂₅)heterocyclic-O—, (C₂-C₂₀)alkenyl-O—, (C₃-C₂₀) cycloalkenyl-O—, (C₂-C₂₀)alkynyl-O—, (C₅-C₂₀)cycloalkynyl-O—, (C₅-C₂₅)aryl-O—, perhalo(C₁-C₂₀)alkyl-O—, (C₁-C₂₀)alkyl-S—, phenyl-S—, (C₃-C₂₀)cycloalkyl-S—, (C₃-C₂₅)heteroaryl-S—, (C₃-C₂₅)heterocyclic-S—, (C₂-C₂₀)alkenyl-S—, (C₃-C₂₀)cycloalkenyl-S—, (C₂-C₂₀)alkynyl-S—, (C₅-C₂₀)cycloalkynyl-S—, (C₅-C₂₅)aryl-S—, perhalo(C₁-C₂₀)alkyl-S—, (C₁-C₂₀)alkyl-SO₂—, phenyl-SO₂—, (C₃-C₂₀)cycloalkyl-SO₂—, (C₁-C₂₀)alkoxy-SO₂—, (C₃-C₂₅)heteroaryl-SO₂—, (C₃-C₂₅)heterocyclic-SO₂—, (C₂-C₂₀)alkenyl-SO₂—, (C₃-C₂₀)cycloalkenyl-SO₂—, (C₂-C₂₀)alkynyl-SO₂—, (C₅-C₂₀)cycloalkynyl-SO₂—, (C₅-C₂₅)aryl-SO₂—, perhalo(C₁-C₂₀)alkyl-SO₂—, H₂N—SO₂—, (C₁-C₂₀)alkyl-NH—SO₂—, phenyl-NH—SO₂—, (C₃-C₂₀)cycloalkyl-NH—SO₂—, (C₁-C₂₀)alkoxy-NH—SO₂—, (C₃-C₂₅)heteroaryl-NH—SO₂—, (C₃-C₂₅)heterocyclic-NH—SO₂—, (C₂-C₂₀)alkenyl-NH—SO₂—, (C₃-C₂₀) cycloalkenyl-NH—SO₂—, (C₂-C₂₀)alkynyl-NH—SO₂—, (C₅-C₂₀)cycloalkynyl-NH—SO₂—, (C₅-C₂₅)aryl-NH—SO₂—, perhalo(C₁-C₂₀)alkyl-NH—SO₂—, {(C₁-C₂₀)alkyl}₂N—SO₂—, {phenyl}₂N—SO₂—, {(C₃-C₂₀)cycloalkyl}₂N—SO₂—, {(C₁-C₂₀)alkoxyl}₂N—SO₂—, {(C₃-C₂₅)heteroaryl}₂N—SO₂—, {(C₃-C₂₅)heterocyclic}₂N—SO₂—, {(C₂-C₂₀)alkenyl}₂N—SO₂—, {(C₂-C₂₀)alkynyl}₂N—SO₂—, {(C₅-C₂₀)cycloalkynyl}₂N—SO₂—, {(C₅-C₂₅)aryl}₂N—SO₂—, {perhalo(C₁-C₂₀)alkyl}₂N—SO₂—, (C₁-C₂₀)alkyl-SO₂—NH—, phenyl-SO₂—NH—, (C₃-C₂₀)cycloalkyl-SO₂—NH—, (C₁-C₂₀)alkoxy-SO₂—NH—, (C₃-C₂₅)heteroaryl-SO₂—NH—, (C₃-C₂₅)heterocyclic-SO₂—NH—, (C₂-C₂₀)alkenyl-SO₂—NH—, (C₃-C₂₀)cycloalkenyl-SO₂—NH—, (C₂-C₂₀)alkynyl-SO₂—NH—, (C₅-C₂₀)cycloalkynyl-SO₂—NH—, (C₅-C₂₅)aryl-SO₂—NH—, perhalo(C₁-C₂₀)alkyl-SO₂—NH—, (C₁-C₂₀)alkyl-NH—, phenyl-NH—, (C₃-C₂₀)cyclo alkyl-NH—, (C₁-C₂₀)alkoxy-NH—, (C₃-C₂₅)heteroaryl-NH—, (C₃-C₂₅)heterocyclic-NH—, (C₂-C₂₀)alkenyl-NH—, (C₃-C₂₀) cycloalkenyl-NH—, (C₂-C₂₀)alkynyl-NH—, (C₅-C₂₀)cycloalkynyl-NH—, (C₅-C₂₅)aryl-NH—, perhalo(C₁-C₂₀)alkyl-NH—, {(C₁-C₂₀)alkyl}₂N—, {phenyl}₂N—, {(C₃-C₂₀)cycloalkyl}₂N—, {(C₁-C₂₀)alkoxy}₂N—, {(C₃-C₂₅)heteroaryl}₂N—, {(C₃-C₂₅)heterocyclic}₂N—, {(C₂-C₂₀)alkenyl}₂N—, {(C₃-C₂₀)cycloalkenyl}₂N—, {(C₂-C₂₀)alkynyl}₂N—, {(C₅-C₂₀)cycloalkynyl}₂N—, {(C₅-C₂₅)aryl}₂N—, {perhalo(C₁-C₂₀)alkyl}₂N—, (C₁-C₂₀)alkyl-(C═O)—NH—, phenyl-(C═O)—NH—, (C₃-C₂₀)cycloalkyl-(C═O)—NH—, (C₁-C₂₀)alkoxy-(C═O)—NH—, (C₃-C₂₅)heteroaryl-(C═O)—NH—, (C₃-C₂₅)heterocyclic-(C═O)—NH—, (C₂-C₂₀)alkenyl-(C═O)—NH—, (C₃-C₂₀)cycloalkenyl-(C═O)—NH—, (C₂-C₂₀)alkynyl-(C═O)—NH—, (C₅-C₂₀)cycloalkynyl-(C═O)—NH—, (C₅-C₂₅)aryl-(C═O)—NH—, perhalo(C₁-C₂₀)alkyl-(C═O)—NH—, (C₁-C₂₀)alkyl-(C═O)—{((C₁-C₂₀)alkyl)N}—, phenyl-(C═O)—{((C₁-C₂₀)alkyl)N}—, (C₃-C₂₀)cycloalkyl-(C═O)—{((C₁-C₂₀)alkyl)N}—, (C₁-C₂₀)alkoxy-(C═O)—{((C₁-C₂₀)alkyl)N}—, (C₃-C₂₅)heteroaryl-(C═O)—{((C₁-C₂₀)alkyl)N}—, (C₃-C₂₅)heterocyclic-(C═O)—{((C₁-C₂₀)alkyl)N}—, (C₂-C₂₀)alkenyl-(C═O)—{((C₁-C₂₀)alkyl)N}—, (C₃-C₂₀)cycloalkenyl-(C═O)—{((C₁-C₂₀)alkyl)N}—, (C₂-C₂₀)alkynyl-(C═O)—{((C₁-C₂₀)alkyl)N}—, (C₅-C₂₀)cycloalkynyl-(C═O)—{((C₁-C₂₀)alkyl)N}—, (C₅-C₂₅)aryl-(C═O)—{((C₁-C₂₀)alkyl)N}—, perhalo(C₁-C₂₀)alkyl-(C═O)—{((C₁-C₂₀)alkyl)N}—, phenyl-(C═O)—NH—, phenyl-(C═O)-{(phenyl)N}—, (C₁-C₂₀)alkyl-(C═O)-{(phenyl)N}—, (C₃-C₂₀)cycloalkyl-(C═O)-{(phenyl)N}—, (C₁-C₂₀)alkoxy-(C═O)-{(phenyl)N}—, (C₃-C₂₅)heteroaryl-(C═O)-{(phenyl)N}—, (C₃-C₂₅)heterocyclic-(C═O)-{(phenyl)N}—, (C₂-C₂₀)alkenyl-(C═O)-{(phenyl)N}—, (C₃-C₂₀)cycloalkenyl-(C═O)-{(phenyl)N}—, (C₂-C₂₀)alkynyl-(C═O)-{(phenyl)N}—, (C₅-C₂₀)cycloalkynyl-(C═O)-{(phenyl)N}—, (C₅-C₂₅)aryl-(C═O)-{(phenyl)N}—, perhalo(C₁-C₂₀)alkyl-(C═O)-{(phenyl)N}—, H₂N(C═O)—, (C₁-C₂₀)alkyl-NH—(C═O)—, phenyl-NH—(C═O)—, (C₃-C₂₀)cycloalkyl-NH—(C═O)—, (C₁-C₂₀)alkoxy-NH—(C═O)—, (C₃-C₂₅)heteroaryl-NH—(C═O)—, (C₃-C₂₅)heterocyclic-NH—(C═O)—, (C₂-C₂₀)alkenyl-NH—(C═O)—, (C₃-C₂₀)cycloalkenyl-NH—(C═O)—, (C₂-C₂₀)alkynyl-NH—(C═O)—, (C₅-C₂₀)cycloalkynyl-NH—(C═O)—, (C₅-C₂₅)aryl-NH—(C═O)—, perhalo(C₁-C₂₀)alkyl-NH—(C═O)—, {C₁-C₂₀)alkyl}₂N—(C═O)—, {phenyl}{(C₁-C₂₀)alkyl}N—(C═O)—, {(C₃-C₂₀)cycloalkyl}{(C₁-C₂₀)alkyl}₂N—(C═O)—, {(C₁-C₂₀)alkoxy}{(C₁-C₂₀)alkyl}N—(C═O)—, {(C₃-C₂₅)heteroaryl}{(C₁-C₂₀)alkyl}N—(C═O)—, {(C₃-C₂₅)heterocyclic}{(C₁-C₂₀)alkyl}N—(C═O)—, {(C₂-C₂₀)alkenyl}{(C₁-C₂₀)alkyl}N—(C═O)—, {(C₃-C₂₀)cycloalkenyl}{(C₁-C₂₀)alkyl}N—(C═O)—, {(C₂-C₂₀)alkynyl}{(C₁-C₂₀)alkyl}N—(C═O)—, {(C₅-C₂₀)cycloalkynyl}{(C₁-C₂₀)alkyl}N—(C═O)—, {(C₅-C₂₅)aryl}{(C₁-C₂₀)alkyl}N—(C═O)—, {perhalo(C₁-C₂₀)alkyl}{(C₁-C₂₀)alkyl}N—(C═O)—, {phenyl}₂N—(C═O)—, {(C₃-C₂₀)cycloalkyl}{phenyl}N—(C═O)—, {(C₁-C₂₀)alkoxy}{phenyl}N—(C═O)—, {(C₃-C₂₅)heteroaryl}{phenyl}N—(C═O)—, {(C₃-C₂₅)heterocyclic}{phenyl}N—(C═O)—, {(C₂-C₂₀)alkenyl}{phenyl}N—(C═O)—, {(C₃-C₂₀)cycloalkenyl}{phenyl}N—(C═O)—, {(C₂-C₂₀)alkynyl}{phenyl}N—(C═O)—, {(C₅-C₂₀)cycloalkynyl}{phenyl}N—(C═O)—, {(C₅-C₂₅)aryl}{phenyl}N—(C═O)—, {perhalo(C₁-C₂₀)alkyl}{phenyl}N—(C═O)—, HO—(C═O)—, (C₁-C₂₀)alkyl-(C═O)—, (C₃-C₂₅)heteroaryl-(C═O)—, (C₃-C₂₅)heterocyclic-(C═O)—, (C₂-C₂₀)alkenyl-(C═O)—, (C₃-C₂₀)cycloalkenyl-(C═O)—, (C₂-C₂₀)alkynyl-(C═O)—, (C₅-C₂₅)aryl-(C═O)—, perhalo(C₁-C₂₀)alkyl-(C═O)—, phenyl-(C═O)—, (C₁-C₂₀)alkyl-O—(C═O)—, (C₃-C₂₅)heteroaryl-O—(C═O)—, (C₃-C₂₅)heterocyclic-O—(C═O)—, (C₂-C₂₀)alkenyl-O—(C═O)—, (C₃-C₂₀) cycloalkenyl-O—(C═O)—, (C₂-C₂₀)alkynyl-O—(C═O)—, (C₅-C₂₅)aryl-O—(C═O)—, perhalo(C₁-C₂₀)alkyl-O—(C═O)—, phenyl-O—(C═O)—, (C₁-C₂₀)alkyl-(C═O)—O—, (C₃-C₂₅)heteroaryl-(C═O)—O—, (C₃-C₂₅)heterocyclic-(C═O)—O—, (C₂-C₂₀)alkenyl-(C═O)—O—, (C₃-C₂₀) cycloalkenyl-(C═O)—O—, (C₂-C₂₀)alkynyl-(C═O)—O—, (C₅-C₂₅)aryl-(C═O)—O—, phenyl-(C═O)—O—, perhalo(C₁-C₂₀)alkyl-(C═O)—O—, and salts thereof; and

wherein R², R³, R⁴, R⁵, R⁶, R⁷, R⁸, R⁹, R¹⁰, and R¹¹ are each independently selected from the group consisting of hydrogen, hydroxy, halo, bromo, chloro, iodo, fluoro, —N₃, —CN, —NC, —SH, —NO₂, —NH₂, (C₁-C₂₀)alkyl, phenyl, (C₃-C₂₀)cyclo alkyl, (C₁-C₂₀)alkoxy, (C₃-C₂₅)heteroaryl, (C₃-C₂₅)heterocyclic, (C₂-C₂₀)alkenyl, (C₃-C₂₀)cycloalkenyl, (C₂-C₂₀)alkynyl, (C₅-C₂₀)cycloalkynyl, (C₅-C₂₅)aryl, perhalo(C₁-C₂₀)alkyl, (C₁-C₂₀)alkyl-O—, phenyl-O—, (C₃-C₂₀)cyclo alkyl-O—, (C₃-C₂₅)heteroaryl-O—, (C₃-C₂₅)heterocyclic-O—, (C₂-C₂₀)alkenyl-O—, (C₃-C₂₀)cycloalkenyl-O—, (C₂-C₂₀)alkynyl-O—, (C₅-C₂₀)cycloalkynyl-O—, (C₅-C₂₅)aryl-O—, perhalo (C₁-C₂₀)alkyl-O—, (C₁-C₂₀)alkyl-S—, phenyl-S—, (C₃-C₂₀)cycloalkyl-S—, (C₃-C₂₅)heteroaryl-S—, (C₃-C₂₅)heterocyclic-S—, (C₂-C₂₀)alkenyl-S—, (C₃-C₂₀)cycloalkenyl-S—, (C₂-C₂₀)alkynyl-S—, (C₅-C₂₀)cycloalkynyl-S—, (C₅-C₂₅)aryl-S—, perhalo(C₁-C₂₀)alkyl-S—, (C₁-C₂₀)alkyl-SO₂—, phenyl-SO₂—, (C₃-C₂₀)cyclo alkyl-SO₂—, (C₁-C₂₀)alkoxy-SO₂—, (C₃-C₂₅)heteroaryl-SO₂—, (C₃-C₂₅)heterocyclic-SO₂—, (C₂-C₂₀)alkenyl-SO₂—, (C₃-C₂₀)cycloalkenyl-SO₂—, (C₂-C₂₀)alkynyl-SO₂—, (C₅-C₂₀)cycloalkynyl-SO₂—, (C₅-C₂₅)aryl-SO₂—, perhalo(C₁-C₂₀)alkyl-SO₂—, H₂N S^{O}₂, (C₁-C₂₀)alkyl-NH—SO₂—, phenyl-NH—SO₂—, (C₃-C₂₀)cycloalkyl-NH—SO₂—, (C₁-C₂₀)alkoxy-NH—SO₂—, (C₃-C₂₅)heteroaryl-NH—SO₂—, (C₃-C₂₅)heterocyclic-NH—SO₂—, (C₂-C₂₀)alkenyl-NH—SO₂—, (C₃-C₂₀)cycloalkenyl-NH—SO₂—, (C₂-C₂₀)alkynyl-NH—SO₂—, (C₅-C₂₀)cycloalkynyl-NH—SO₂—, (C₅-C₂₅)aryl-NH—SO₂—, perhalo(C₁-C₂₀)alkyl-NH—SO₂—, {(C₁-C₂₀)alkyl}₂N—SO₂—, {phenyl}₂N—SO₂—, {(C₃-C₂₀)cycloalkyl}₂N—SO₂—, {(C₁-C₂₀)alkoxy}₂N—SO₂—, {(C₃-C₂₅)heteroaryl}₂N—SO₂—, {(C₃-C₂₅)heterocyclic}₂N—SO₂—, {(C₂-C₂₀)alkenyl}₂N—SO₂—, {(C₂-C₂₀)alkynyl}₂N—SO₂—, {(C₅-C₂₀)cycloalkynyl}₂N—SO₂—, {(C₅-C₂₅)aryl}₂N—SO₂—, {perhalo (C₁-C₂₀)alkyl}₂N—SO₂—, (C₁-C₂₀)alkyl-SO₂—NH—, phenyl-SO₂—NH—, (C₃-C₂₀)cycloalkyl-SO₂—NH—, (C₁-C₂₀)alkoxy-SO₂—NH—, (C₃-C₂₅)heteroaryl-SO₂—NH—, (C₃-C₂₅)heterocyclic-SO₂—NH—, (C₂-C₂₀)alkenyl-SO₂—NH—, (C₃-C₂₀)cycloalkenyl-SO₂—NH—, (C₂-C₂₀)alkynyl-SO₂—NH—, (C₅-C₂₀)cycloalkynyl-SO₂—NH—, (C₅-C₂₅)aryl-SO₂—NH—, perhalo(C₁-C₂₀)alkyl-SO₂—NH—, (C₁-C₂₀)alkyl-NH—, phenyl-NH—, (C₃-C₂₀)cycloalkyl-NH—, (C₁-C₂₀)alkoxy-NH—, (C₃-C₂₅)heteroaryl-NH—, (C₃-C₂₅)heterocyclic-NH—, (C₂-C₂₀)alkenyl-NH—, (C₃-C₂₀)cycloalkenyl-NH—, (C₂-C₂₀)alkynyl-NH—, (C₅-C₂₀)cycloalkynyl-NH—, (C₅-C₂₅)aryl-NH—, perhalo(C₁-C₂₀)alkyl-NH—, {(C₁-C₂₀)alkyl}₂N—, {phenyl}₂N—, {(C₃-C₂₀)cycloalkyl}₂N—, {(C₁-C₂₀)alkoxy}₂N—, {(C₃-C₂₅)heteroaryl}₂N—, {(C₃-C₂₅)heterocyclic}₂N—, {(C₂-C₂₀)alkenyl}₂N—, {(C₃-C₂₀)cycloalkenyl}₂N—, {(C₂-C₂₀)alkynyl}₂N—, {(C₅-C₂₀)cycloalkynyl}₂N—, {(C₅-C₂₅)aryl}₂N—, {perhalo(C₁-C₂₀)alkyl}₂N—, (C₁-C₂₀)alkyl-(C═O)—NH—, phenyl-(C═O)—NH—, (C₃-C₂₀)cycloalkyl-(C═O)—NH—, (C₁-C₂₀)alkoxy-(C═O)—NH—, (C₃-C₂₅)heteroaryl-(C═O)—NH—, (C₃-C₂₅)heterocyclic-(C═O)—NH—, (C₂-C₂₀)alkenyl-(C═O)—NH—, (C₃-C₂₀)cycloalkenyl-(C═O)—NH—, (C₂-C₂₀)alkynyl-(C═O)—NH—, (C₅-C₂₀)cycloalkynyl-(C═O)—NH—, (C₅-C₂₅)aryl-(C═O)—NH—, perhalo(C₁-C₂₀)alkyl-(C═O)—NH—, (C₁-C₂₀)alkyl-(C═O)—{((C₁-C₂₀)alkyl)N}—, phenyl-(C═O)—{((C₁-C₂₀)alkyl)N}—, (C₃-C₂₀)cycloalkyl-(C═O)—{((C₁-C₂₀)alkyl)N}—, (C₁-C₂₀)alkoxy-(C═O)—{((C₁-C₂₀)alkyl)N}—, (C₃-C₂₅)heteroaryl-(C═O)—{((C₁-C₂₀)alkyl)N}—, (C₃-C₂₅)heterocyclic-(C═O)—{((C₁-C₂₀)alkyl)N}—, (C₂-C₂₀)alkenyl-(C═O)—{((C₁-C₂₀)alkyl)N}—, (C₃-C₂₀)cycloalkenyl-(C═O)—{((C₁-C₂₀)alkyl)N}—, (C₂-C₂₀)alkynyl-(C═O)—{((C₁-C₂₀)alkyl)N}—, (C₅-C₂₀)cycloalkynyl-(C═O)—{((C₁-C₂₀)alkyl)N}—, (C₅-C₂₅)aryl-(C═O)—{((C₁-C₂₀)alkyl)N}—, perhalo(C₁-C₂₀)alkyl-(C═O)—{((C₁-C₂₀)alkyl)N}—, phenyl-(C═O)—NH—, phenyl-(C═O)-{(phenyl)N}—, (C₁-C₂₀)alkyl-(C═O)-{(phenyl)N}—, (C₃-C₂₀)cycloalkyl-(C═O)-{(phenyl)N}—, (C₁-C₂₀)alkoxy-(C═O)-{(phenyl)N}—, (C₃-C₂₅)heteroaryl-(C═O)—{(phenyl)N}—, (C₃-C₂₅)heterocyclic-(C═O)-{(phenyl)N}—, (C₂-C₂₀)alkenyl-(C═O)-{(phenyl)N}—, (C₃-C₂₀)cycloalkenyl-(C═O)-{(phenyl)N}—, (C₂-C₂₀)alkynyl-(C═O)-{(phenyl)N}—, (C₅-C₂₀)cycloalkynyl-(C═O)-{(phenyl)N}—, (C₅-C₂₅)aryl-(C═O)-{(phenyl)N}—, perhalo(C₁-C₂₀)alkyl-(C═O)-{(phenyl)N}—, H₂N(C═O)—, (C₁-C₂₀)alkyl-NH—(C═O)—, phenyl-NH—(C═O)—, (C₃-C₂₀)cycloalkyl-NH—(C═O)—, (C₁-C₂₀)alkoxy-NH—(C═O)—, (C₃-C₂₅)heteroaryl-NH—(C═O)—, (C₃-C₂₅)heterocyclic-NH—(C═O)—, (C₂-C₂₀)alkenyl-NH—(C═O)—, (C₃-C₂₀) cycloalkenyl-NH—(C═O)—, (C₂-C₂₀)alkynyl-NH—(C═O)—, (C₅-C₂₀)cycloalkynyl-NH—(C═O)—, (C₅-C₂₅)aryl-NH—(C═O)—, perhalo(C₁-C₂₀)alkyl-NH—(C═O)—, {C₁-C₂₀)alkyl}₂N—(C═O)—, {phenyl}{(C₁-C₂₀)alkyl}N—(C═O)—, {(C₃-C₂₀)cycloalkyl}{(C₁-C₂₀)alkyl}N—(C═O)—, {(C₁-C₂₀)alkoxy}{(C₁-C₂₀)alkyl}N—(C═O)—, {(C₃-C₂₅)heteroaryl}{(C₁-C₂₀)alkyl}N—(C═O)—, {(C₃-C₂₅)heterocyclic}{(C₁-C₂₀)alkyl}-N—(C═O)—, {(C₂-C₂₀)alkenyl}{(C₁-C₂₀)alkyl}N—(C═O)—, {(C₃-C₂₀)cycloalkenyl}{(C₁-C₂₀)alkyl}N—(C═O)—, {(C₂-C₂₀)alkynyl}{(C₁-C₂₀)alkyl}N—(C═O)—, {(C₅-C₂₀)cycloalkynyl}{(C₁-C₂₀)alkyl}N—(C═O)—, {(C₅-C₂₅)aryl}{(C₁-C₂₀)alkyl}N—(C═O)—, {perhalo(C₁-C₂₀)alkyl}{(C₁-C₂₀)alkyl}N—(C═O)—, {phenyl}₂N—(C═O)—, {(C₃-C₂₀)cycloalkyl}{phenyl}N—(C═O)—, {(C₁-C₂₀)alkoxy}{phenyl}N—(C═O)—, {(C₃-C₂₅)heteroaryl}{phenyl}N—(C═O)—, {(C₃-C₂₅)heterocyclic}{phenyl}N—(C═O)—, {(C₂-C₂₀)alkenyl}{phenyl}N—(C═O)—, {(C₃-C₂₀)cycloalkenyl}{phenyl}N—(C═O)—, {(C₂-C₂₀)alkynyl}{phenyl}N—(C═O)—, {(C₅-C₂₀)cycloalkynyl}{phenyl}N—(C═O)—, {(C₅-C₂₅)aryl}{phenyl}N—(C═O)—, {perhalo(C₁-C₂₀)alkyl}{phenyl}N—(C═O)—, HO—(C═O)—, (C₁-C₂₀)alkyl-(C═O)—, (C₃-C₂₅)heteroaryl-(C═O)—, (C₃-C₂₅)heterocyclic-(C═O)—, (C₂-C₂₀)alkenyl-(C═O)—, (C₃-C₂₀)cycloalkenyl-(C═O)—, (C₂-C₂₀)alkynyl-(C═O)—, (C₅-C₂₅)aryl-(C═O)—, perhalo(C₁-C₂₀)alkyl-(C═O)—, phenyl-(C═O)—, (C₁-C₂₀)alkyl-O—(C═O)—, (C₃-C₂₅)heteroaryl-O—(C═O)—, (C₃-C₂₅)heterocyclic-O—(C═O)—, (C₂-C₂₀)alkenyl-O—(C═O)—, (C₃-C₂₀) cycloalkenyl-O—(C═O)—, (C₂-C₂₀)alkynyl-O—(C═O)—, (C₅-C₂₅)aryl-O—(C═O)—, perhalo(C₁-C₂₀)alkyl-O—(C═O)—, phenyl-O—(C═O)—, (C₁-C₂₀)alkyl-(C═O)—O—, (C₃-C₂₅)heteroaryl-(C═O)—O—, (C₃-C₂₅)heterocyclic-(C═O)—O—, (C₂-C₂₀)alkenyl-(C═O)—O—, (C₃-C₂₀) cycloalkenyl-(C═O)—O—, (C₂-C₂₀)alkynyl-(C═O)—O—, (C₅-C₂₅)aryl-(C═O)—O—, phenyl-(C═O)—O—, perhalo(C₁-C₂₀)alkyl-(C═O)—O—, and salts thereof;

wherein each of the aforesaid (C₁-C₂₀)alkyl, phenyl, (C₃-C₂₀)cycloalkyl, (C₁-C₂₀)alkoxy, (C₃-C₂₅)heteroaryl, (C₃-C₂₅)heterocyclic, (C₂-C₂₀)alkenyl, (C₃-C₂₀)cycloalkenyl, (C₂-C₂₀)alkynyl, (C₅-C₂₀)cycloalkynyl, and (C₅-C₂₅)aryl groups (for said R², R³, R⁴, R⁵, R⁶, R⁷, R⁸, R⁹, R¹⁰, and R¹¹ groups) may be optionally and independently substituted by one to four moieties selected from the group consisting of hydroxy, halo, bromo, chloro, iodo, fluoro, —N₃, —CN, —NC, —SH, —NO₂, —NH₂, (C₁-C₂₀)alkyl, phenyl, (C₃-C₂₀)cycloalkyl, (C₁-C₂₀)alkoxy, (C₃-C₂₅)heteroaryl, (C₃-C₂₅)heterocyclic, (C₂-C₂₀)alkenyl, (C₃-C₂₀)cycloalkenyl, (C₂-C₂₀)alkynyl, (C₅-C₂₀)cycloalkynyl, (C₅-C₂₅)aryl, perhalo(C₁-C₂₀)alkyl, (C₁-C₂₀)alkyl-O—, phenyl-O—, (C₃-C₂₀)cycloalkyl-O—, (C₃-C₂₅)heteroaryl-O—, (C₃-C₂₅)heterocyclic-O—, (C₂-C₂₀)alkenyl-O—, (C₃-C₂₀) cycloalkenyl-O—, (C₂-C₂₀)alkynyl-O—, (C₅-C₂₀)cycloalkynyl-O—, (C₅-C₂₅)aryl-O—, perhalo(C₁-C₂₀)alkyl-O—, (C₁-C₂₀)alkyl-S—, phenyl-S—, (C₃-C₂₀)cycloalkyl-S—, (C₃-C₂₅)heteroaryl-S—, (C₃-C₂₅)heterocyclic-S—, (C₂-C₂₀)alkenyl-S—, (C₃-C₂₀)cycloalkenyl-S—, (C₂-C₂₀)alkynyl-S—, (C₅-C₂₀)cycloalkynyl-S—, (C₅-C₂₅)aryl-S—, perhalo(C₁-C₂₀)alkyl-S—, (C₁-C₂₀)alkyl-SO₂—, phenyl-SO₂—, (C₃-C₂₀)cycloalkyl-SO₂—, (C₁-C₂₀)alkoxy-SO₂—, (C₃-C₂₅)heteroaryl-SO₂—, (C₃-C₂₅)heterocyclic-SO₂—, (C₂-C₂₀)alkenyl-SO₂—, (C₃-C₂₀)cycloalkenyl-SO₂—, (C₂-C₂₀)alkynyl-SO₂—, (C₅-C₂₀)cycloalkynyl-SO₂—, (C₅-C₂₅)aryl-SO₂—, perhalo(C₁-C₂₀)alkyl-SO₂—, H₂N—SO₂—, (C₁-C₂₀)alkyl-NH—SO₂—, phenyl-NH—SO₂—, (C₃-C₂₀)cycloalkyl-NH—SO₂—, (C₁-C₂₀)alkoxy-NH—SO₂—, (C₃-C₂₅)heteroaryl-NH—SO₂—, (C₃-C₂₅)heterocyclic-NH—SO₂—, (C₂-C₂₀)alkenyl-NH—SO₂—, (C₃-C₂₀) cycloalkenyl-NH—SO₂—, (C₂-C₂₀)alkynyl-NH—SO₂—, (C₅-C₂₀)cycloalkynyl-NH—SO₂—, (C₅-C₂₅)aryl-NH—SO₂—, perhalo(C₁-C₂₀)alkyl-NH—SO₂—, {(C₁-C₂₀)alkyl}₂N—SO₂—, {phenyl}₂N—SO₂—, {(C₃-C₂₀)cycloalkyl}₂N—SO₂—, {(C₁-C₂₀)alkoxyl}₂N—SO₂—, {(C₃-C₂₅)heteroaryl}₂N—SO₂—, {(C₃-C₂₅)heterocyclic}₂N—SO₂—, {(C₂-C₂₀)alkenyl}₂N—SO₂—, {(C₂-C₂₀)alkynyl}₂N—SO₂—, {(C₅-C₂₀)cycloalkynyl}₂N—SO₂—, {(C₅-C₂₅)aryl}₂N—SO₂—, {perhalo(C₁-C₂₀)alkyl}₂N—SO₂—, (C₁-C₂₀)alkyl-SO₂—NH—, phenyl-SO₂—NH—, (C₃-C₂₀)cycloalkyl-SO₂—NH—, (C₁-C₂₀)alkoxy-SO₂—NH—, (C₃-C₂₅)heteroaryl-SO₂—NH—, (C₃-C₂₅)heterocyclic-SO₂—NH—, (C₂-C₂₀)alkenyl-SO₂—NH—, (C₃-C₂₀)cycloalkenyl-SO₂—NH—, (C₂-C₂₀)alkynyl-SO₂—NH—, (C₅-C₂₀)cycloalkynyl-SO₂—NH—, (C₅-C₂₅)aryl-SO₂—NH—, perhalo(C₁-C₂₀)alkyl-SO₂—NH—, (C₁-C₂₀)alkyl-NH—, phenyl-NH—, (C₃-C₂₀)cyclo alkyl-NH—, (C₁-C₂₀)alkoxy-NH—, (C₃-C₂₅)heteroaryl-NH—, (C₃-C₂₅)heterocyclic-NH—, (C₂-C₂₀)alkenyl-NH—, (C₃-C₂₀) cycloalkenyl-NH—, (C₂-C₂₀)alkynyl-NH—, (C₅-C₂₀)cycloalkynyl-NH—, (C₅-C₂₅)aryl-NH—, perhalo(C₁-C₂₀)alkyl-NH—, {(C₁-C₂₀)alkyl}₂N—, {phenyl}₂N—, {(C₃-C₂₀)cycloalkyl}₂N—, {(C₁-C₂₀)alkoxyl}₂N—, {(C₃-C₂₅)heteroaryl}₂N—, {(C₃-C₂₅)heterocyclic}₂N—, {(C₂-C₂₀)alkenyl}₂N—, {(C₃-C₂₀)cycloalkenyl}₂N—, {(C₂-C₂₀)alkynyl}₂N—, {(C₅-C₂₀)cycloalkynyl}₂N—, {(C₅-C₂₅)aryl}₂N—, {perhalo(C₁-C₂₀)alkyl}₂N—, (C₁-C₂₀)alkyl-(C═O)—NH—, phenyl-(C═O)—NH—, (C₃-C₂₀)cycloalkyl-(C═O)—NH—, (C₁-C₂₀)alkoxy-(C═O)—NH—, (C₃-C₂₅)heteroaryl-(C═O)—NH—, (C₃-C₂₅)heterocyclic-(C═O)—NH—, (C₂-C₂₀)alkenyl-(C═O)—NH—, (C₃-C₂₀)cycloalkenyl-(C═O)—NH—, (C₂-C₂₀)alkynyl-(C═O)—NH—, (C₅-C₂₀)cycloalkynyl-(C═O)—NH—, (C₅-C₂₅)aryl-(C═O)—NH—, perhalo(C₁-C₂₀)alkyl-(C═O)—NH—, (C₁-C₂₀)alkyl-(C═O)—{((C₁-C₂₀)alkyl)N}—, phenyl-(C═O)—{((C₁-C₂₀)alkyl)N}—, (C₃-C₂₀)cycloalkyl-(C═O)—{((C₁-C₂₀)alkyl)N}—, (C₁-C₂₀)alkoxy-(C═O)—{((C₁-C₂₀)alkyl)N}—, (C₃-C₂₅)heteroaryl-(C═O)—{((C₁-C₂₀)alkyl)N}—, (C₃-C₂₅)heterocyclic-(C═O)—{((C₁-C₂₀)alkyl)N}—, (C₂-C₂₀)alkenyl-(C═O)—{((C₁-C₂₀)alkyl)N}—, (C₃-C₂₀)cycloalkenyl-(C═O)—{((C₁-C₂₀)alkyl)N}—, (C₂-C₂₀)alkynyl-(C═O)—{((C₁-C₂₀)alkyl)N}—, (C₅-C₂₀)cycloalkynyl-(C═O)—{((C₁-C₂₀)alkyl)N}—, (C₅-C₂₅)aryl-(C═O)—{((C₁-C₂₀)alkyl)N}—, perhalo(C₁-C₂₀)alkyl-(C═O)—{((C₁-C₂₀)alkyl)N}—, phenyl-(C═O)—NH—, phenyl-(C═O)-{(phenyl)N}—, (C₁-C₂₀)alkyl-(C═O)-{(phenyl)N}—, (C₃-C₂₀)cycloalkyl-(C═O)-{(phenyl)N}—, (C₁-C₂₀)alkoxy-(C═O)-{(phenyl)N}—, (C₃-C₂₅)heteroaryl-(C═O)-{(phenyl)N}—, (C₃-C₂₅)heterocyclic-(C═O)-{(phenyl)N}—, (C₂-C₂₀)alkenyl-(C═O)-{(phenyl)N}—, (C₃-C₂₀)cycloalkenyl-(C═O)-{(phenyl)N}—, (C₂-C₂₀)alkynyl-(C═O)-{(phenyl)N}—, (C₅-C₂₀)cycloalkynyl-(C═O)-{(phenyl)N}—, (C₅-C₂₅)aryl-(C═O)-{(phenyl)N}—, perhalo(C₁-C₂₀)alkyl-(C═O)-{(phenyl)N}—, H₂N(C═O)—, (C₁-C₂₀)alkyl-NH—(C═O)—, phenyl-NH—(C═O)—, (C₃-C₂₀)cycloalkyl-NH—(C═O)—, (C₁-C₂₀)alkoxy-NH—(C═O)—, (C₃-C₂₅)heteroaryl-NH—(C═O)—, (C₃-C₂₅)heterocyclic-NH—(C═O)—, (C₂-C₂₀)alkenyl-NH—(C═O)—, (C₃-C₂₀)cycloalkenyl-NH—(C═O)—, (C₂-C₂₀)alkynyl-NH—(C═O)—, (C₅-C₂₀)cycloalkynyl-NH—(C═O)—, (C₅-C₂₅)aryl-NH—(C═O)—, perhalo(C₁-C₂₀)alkyl-NH—(C═O)—, {C₁-C₂₀)alkyl}₂N—(C═O)—, {phenyl}{(C₁-C₂₀)alkyl}N—(C═O)—, {(C₃-C₂₀)cycloalkyl}{(C₁-C₂₀)alkyl}N—(C═O)—, {(C₁-C₂₀)alkoxy}{(C₁-C₂₀)alkyl}N—(C═O)—, {(C₃-C₂₅)heteroaryl}{(C₁-C₂₀)alkyl}N—(C═O)—, {(C₃-C₂₅)heterocyclic}{(C₁-C₂₀)alkyl}N—(C═O)—, {(C₂-C₂₀)alkenyl}{(C₁-C₂₀)alkyl}N—(C═O)—, {(C₃-C₂₀)cycloalkenyl}{(C₁-C₂₀)alkyl}N—(C═O)—, {(C₂-C₂₀)alkynyl}{(C₁-C₂₀)alkyl}N—(C═O)—, {(C₅-C₂₀)cycloalkynyl}{(C₁-C₂₀)alkyl}N—(C═O)—, {(C₅-C₂₅)aryl}{(C₁-C₂₀)alkyl}N—(C═O)—, {perhalo(C₁-C₂₀)alkyl}{(C₁-C₂₀)alkyl}N—(C═O)—, {phenyl}₂N—(C═O)—, {(C₃-C₂₀)cycloalkyl}{phenyl}N—(C═O)—, {(C₁-C₂₀)alkoxy}{phenyl}N—(C═O)—, {(C₃-C₂₅)heteroaryl}{phenyl}N—(C═O)—, {(C₃-C₂₅)heterocyclic}{phenyl}N—(C═O)—, {(C₂-C₂₀)alkenyl}{phenyl}N—(C═O)—, {(C₃-C₂₀)cycloalkenyl}{phenyl}N—(C═O)—, {(C₂-C₂₀)alkynyl}{phenyl}N—(C═O)—, {(C₅-C₂₀)cycloalkynyl}{phenyl}N—(C═O)—, {(C₅-C₂₅)aryl}{phenyl}N—(C═O)—, {perhalo(C₁-C₂₀)alkyl}{phenyl}N—(C═O)—, HO—(C═O)—, (C₁-C₂₀)alkyl-(C═O)—, (C₃-C₂₅)heteroaryl-(C═O)—, (C₃-C₂₅)heterocyclic-(C═O)—, (C₂-C₂₀)alkenyl-(C═O)—, (C₃-C₂₀)cycloalkenyl-(C═O)—, (C₂-C₂₀)alkynyl-(C═O)—, (C₅-C₂₅)aryl-(C═O)—, perhalo(C₁-C₂₀)alkyl-(C═O)—, phenyl-(C═O)—, (C₁-C₂₀)alkyl-O—(C═O)—, (C₃-C₂₅)heteroaryl-O—(C═O)—, (C₃-C₂₅)heterocyclic-O—(C═O)—, (C₂-C₂₀)alkenyl-O—(C═O)—, (C₃-C₂₀) cycloalkenyl-O—(C═O)—, (C₂-C₂₀)alkynyl-O—(C═O)—, (C₅-C₂₅)aryl-O—(C═O)—, perhalo(C₁-C₂₀)alkyl-O—(C═O)—, phenyl-O—(C═O)—, (C₁-C₂₀)alkyl-(C═O)—O—, (C₃-C₂₅)heteroaryl-(C═O)—O—, (C₃-C₂₅)heterocyclic-(C═O)—O—, (C₂-C₂₀)alkenyl-(C═O)—O—, (C₃-C₂₀) cycloalkenyl-(C═O)—O—, (C₂-C₂₀)alkynyl-(C═O)—O—, (C₅-C₂₅)aryl-(C═O)—O—, phenyl-(C═O)—O—, perhalo(C₁-C₂₀)alkyl-(C═O)—O—, and salts thereof;

and wherein two independently chosen R², R³, R⁴, R⁵, R⁶, R⁷, R⁸, R⁹, R¹⁰ and R¹¹, alkyl-containing groups may be taken together with any atom to which they are attached to form a three to forty membered cyclic, heterocyclic or heteroaryl ring.

In the present application, “Ghy” is a guanylhydrazone group; GhyCH— is NH₂C(═NH)—NH—N═CH—; and GhyCH₃— is NH₂C(═NH)—NH—N═CCH₃—.

In another embodiment, Z has the formula:

-(A¹)ₐ-(CR²R³)ₓ-Qₘ-(CR⁴R⁵)_{y}-Tₙ-(CR⁶R⁷)_{z}-(A²)_{b}-

wherein Q and T are each independently selected from the group consisting R¹⁰(CO)NR¹¹—, —(CO)NR¹⁰—, —NR¹⁰(CO)—, —NR¹⁰—, salts thereof, —O—, optionally substituted alkylene, optionally substituted arylene, optionally substituted heteroarylene, and combinations thereof;

wherein A¹, R², R³, R⁴, R⁵, R⁶, R⁷, A², R¹⁰, and R¹¹ are defined herein;

wherein if substituted, the alkylene, arylene, and heteroarylene, are each independently substituted with 0 to 4 groups selected from the group consisting of H, halogen, OR, NR¹R^{1′}, NR¹CO, CONR¹, COR¹, SR¹, SO₂R¹, SO₂NR¹, SOR¹, alkyl, aryl, heteroalkyl, and heteroaryl, salts thereof, and combinations thereof;

and wherein R¹ and R^{1′} being each independently selected from the group including alkyl, aryl, heteroalkyl, and heteroaryl.

In one embodiment, Z is a C₁-C₂₀ alkylene, which may be branched or unbranched, saturated or unsaturated, substituted or unsubstituted, and which may have one or more carbon atoms replaced by one or more heteroatoms selected from the group consisting of oxygen, nitrogen, sulfur, and a combination thereof. This includes alkylenes having 1, 2, 3, 4, 5, 6, 7, 8, 9, 10, 11, 12, 13, 14, 15, 16, 17, 18, 19, and 20 carbons.

In another embodiment, Z is a branched C₁-C₂₀ alkylene.

In another embodiment, Z is an unbranched C₁-C₂₀ alkylene.

In another embodiment, Z is a saturated C₁-C₂₀ alkylene.

In another embodiment, Z is an unsaturated C₁-C₂₀ alkylene.

In another embodiment, Z is an unsubstituted C₁-C₂₀ alkylene.

In another embodiment, Z is a substituted C₁-C₂₀ alkylene.

In another embodiment, Z is a C₁-C₂₀ alkylene in which one or more carbons is replaced with one or more heteroatoms selected from the group including oxygen, nitrogen, sulfur and a combination thereof.

In one embodiment, Z is a saturated or unsaturated, substituted or unsubstituted C₃-C₂₀ cycloalkylene, and which may have one or more carbon atoms replaced by one or more heteroatoms selected from the group consisting of oxygen, nitrogen, sulfur, and a combination thereof. This includes cycloalkylenes having 3, 4, 5, 6, 7, 8, 9, 10, 11, 12, 13, 14, 15, 16, 17, 18, 19, and 20 carbons.

In another embodiment, Z is a saturated C₃-C₂₀ cycloalkylene.

In another embodiment, Z is an unsaturated C₃-C₂₀ cycloalkylene.

In another embodiment, Z is an unsubstituted C₃-C₂₀ cycloalkylene.

In another embodiment, Z is a substituted C₃-C₂₀ cycloalkylene.

In another embodiment, Z is a C₃-C₂₀ cycloalkylene in which one or more carbons is replaced with one or more heteroatoms selected from the group including oxygen, nitrogen, sulfur and a combination thereof.

In one embodiment, Z is a substituted or unsubstituted C₅-C₂₅ arylene, wherein one or more carbon atoms in the cycloalkylene and arylene may be replaced with one or more heteroatoms selected from the group consisting of nitrogen, oxygen, sulfur, and a combination thereof. This includes arylenes having 6, 7, 8, 9, 10, 11, 12, 13, 14, 15, 16, 17, 18, 19, 20, 21, 22, 23, 24, and 25 carbons.

In another embodiment, Z is a substituted C₅-C₂₅ arylene.

In another embodiment, Z is an unsubstituted C₅-C₂₅ arylene.

In another embodiment, Z is a C₅-C₂₅ arylene in which one or more carbons is replaced with one or more heteroatoms selected from the group including oxygen, nitrogen, sulfur and a combination thereof.

In one embodiment, Z is an —NR⁸(CO)NR⁹— group, optionally in the salt form, wherein the R groups are both hydrogen.

In another embodiment, Z is a —(C₆H₄)— group.

In another embodiment, Z is a —(CH₂)ₚ— group, wherein p is 1, 2, 3, 4, 5, 6, 7, 8, 9, or 10.

In another embodiment, Z is a —(C₅H₃N)— group.

In another embodiment, Z is a —O—(CH₂)ₚ—O— group, wherein p is 1, 2, 3, 4, 5, 6, 7, 8, 9, or 10.

In another embodiment, Z is a -A-(CH₂)ₚ-A- group, wherein p is 1, 2, 3, 4, 5, 6, 7, 8, 9, or 10, and wherein the A's are each independently —NH(CO)—, —(CO)NH—, or —NH(CO)NH— groups.

In another embodiment, Z is a -A-(C₆H₄)-A- wherein the A's are each independently —CO—, —NH(CO)—, —(CO)NH—, or —NH(CO)NH— groups.

In another embodiment, Z is —O—(C₆H₄)—O—, wherein the two “—O—” groups are para to each other about the phenylene ring.

In another embodiment, Z is —O—(C₆H₄)—O—, wherein the two “—O—” groups are meta to each other about the phenylene ring.

In another embodiment, Z is —O—CH₂—CH₂—O—CH₂—CH₂—O—.

In another embodiment, Z is a group having the formula:

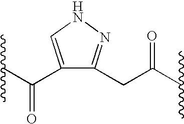

In another embodiment, Z is a group having the formula:

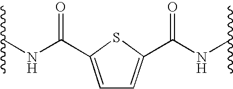

In another embodiment, Z is a group having the formula:

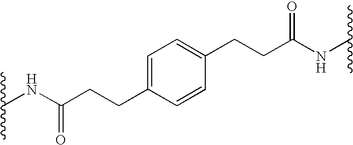

In another embodiment, Z is a group having the formula:

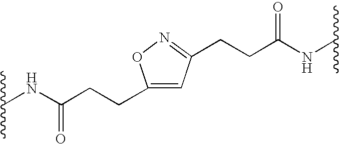

In another embodiment. Z is a group having the formula:

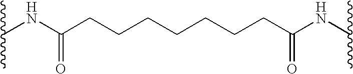

In another embodiment, Z is a group having the formula:

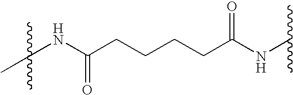

In another embodiment, Z is a group having the formula:

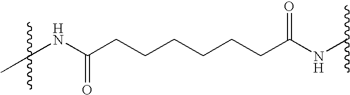

In another embodiment, Z is a group having the formula:

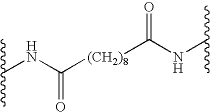

In one embodiment, the compound includes the structure:

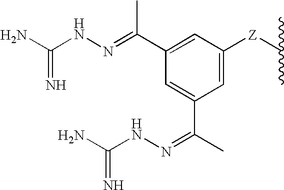

In one embodiment, the compound includes the structure:

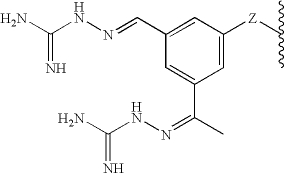

In one embodiment, the compound includes the structure:

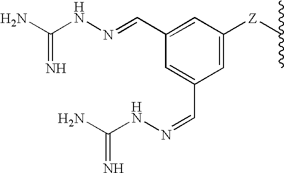

In one embodiment, the compound includes the structure:

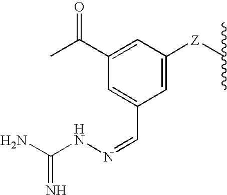

In one embodiment, the compound includes the structure:

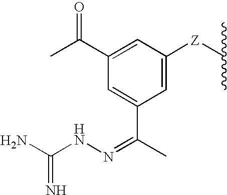

In one embodiment, the compound includes the structure:

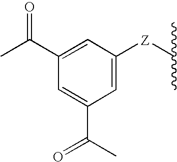

In one embodiment, the compound includes the structure:

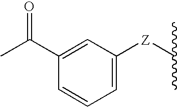

In one embodiment, the compound includes the structure:

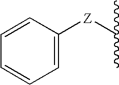

In one embodiment, the compound includes the structure:

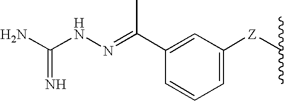

In one embodiment, the compound includes the structure:

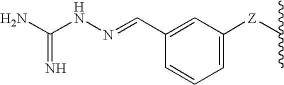

In the compound, X¹, X², X³, and X⁴ may each individually adopt the ortho, meta or para position on the phenylene ring relative to the Z group. In another embodiment, the X¹, X², X³, and X⁴ are meta or para to the Z group. In another embodiment, the non-H X¹, X², X³, and X⁴ groups are meta to both the Z group and to each other.

As used herein, the formula “—NH(CO)—” includes the “—(CO)NH—” isomer.

In one embodiment, at least one of X¹, X², X³ and X⁴ is GhyCH or GhyCCH₃—, X¹ and X² are not simultaneously H, and X³ and X⁴ are not simultaneously H.

In another embodiment, X¹, X², X³, and X⁴ are selected from the group including GhyCH— or GhyCCH₃—.

In another embodiment, X¹, X², X³, and X⁴ are selected from the group including GhyCH—, GhyCCH₃—, or CH₃CO—.

In another embodiment, X¹, X², X³, and X⁴ are each GhyCH—.

In another embodiment, the X¹, X², X³, and X⁴ are each GhyCCH₃—.

In another embodiment, the X¹, X², X³, and X⁴ are each CH₃CO—.

In another embodiment, at least one of X¹, X², X³, and X⁴ is CH₃CO—.

In one embodiment, the compound is in the salt form.

In one embodiment, Z has the formula:

-(A¹)ₐ-(CR²R³)ₓ-Qₘ-(CR⁴R⁵)_{y}-Tₙ-(CR⁶R⁷)_{z}-(A²)_{b}-;

wherein each of the variables a, m, n, and b are equal to 1; and the sum of the variables x, y and z does not exceed 12;

and wherein Q, T, A¹, R², R³, R⁴, R⁵, R⁶, R⁷, A², R¹⁰, and R¹¹ are defined herein.

In one embodiment, Z has the formula:

-(A¹)ₐ-(CR²R³)ₓ-Qₘ-(CR⁴R⁵)_{y}-Tₙ-(CR⁶R⁷)_{z}-(A²)_{b}-;

wherein each of the variables a, m, n, and b are equal to 1; and the sum of the variables x, y and z does not exceed 12;

wherein Q and T are each independently selected from the group consisting R¹⁰(CO)NR¹¹—, —(CO)NR¹⁰—, —NR¹⁰(CO)—, —NR¹⁰—, salts thereof, —O—, optionally substituted alkylene, optionally substituted arylene, optionally substituted heteroarylene, and combinations thereof;

and wherein A¹, R², R³, R⁴, R⁵, R⁶, R⁷, A², R¹⁰, and R¹¹ are defined herein.

In one embodiment, said alkylene, cycloalkylene or arylene in said Q and/or T are each independently substituted with one or more substituent groups selected from the group consisting of hydroxy, halo, bromo, chloro, iodo, fluoro, —N₃, —CN, —NC, —SH, NO₂, —NH₂, salts thereof, and combinations thereof.

In another embodiment, said alkylene, cycloalkylene or arylene in said Q and/or T are each independently substituted with one or more substituent groups selected from the group consisting of (C₁-C₂₀)alkyl, phenyl, (C₃-C₂₀)cycloalkyl, (C₁-C₂₀)alkoxy, (C₃-C₂₅)heteroaryl, (C₃-C₂₅)heterocyclic, (C₂-C₂₀)alkenyl, (C₃-C₂₀)cycloalkenyl, (C₂-C₂₀)alkynyl, (C₅-C₂₀)cycloalkynyl, (C₅-C₂₅)aryl, perhalo(C₁-C₂₀)alkyl, and a combination thereof.

In another embodiment, said alkylene, cycloalkylene or arylene in said Q and/or T are each independently substituted with one or more substituent groups selected from the group consisting of (C₁-C₂₀)alkyl-O—, phenyl-O—, (C₃-C₂₀)cycloalkyl-O—, (C₃-C₂₅)heteroaryl-O—, (C₃-C₂₅)heterocyclic-O—, (C₂-C₂₀)alkenyl-O—, (C₃-C₂₀) cycloalkenyl-O—, (C₂-C₂₀)alkynyl-O—, (C₅-C₂₀)cycloalkynyl-O—, (C₅-C₂₅)aryl-O—, perhalo(C₁-C₂₀)alkyl-O—, and a combination thereof.

In another embodiment, said alkylene, cycloalkylene or arylene in said Q and/or T are each independently substituted with one or more substituent groups selected from the group consisting of (C₁-C₂₀)alkyl-S—, phenyl-S—, (C₃-C₂₀)cycloalkyl-S—, (C₃-C₂₅)heteroaryl-S—, (C₃-C₂₅)heterocyclic-S—, (C₂-C₂₀)alkenyl-S—, (C₃-C₂₀)cycloalkenyl-S—, (C₂-C₂₀)alkynyl-S—, (C₅-C₂₀)cycloalkynyl-S—, (C₅-C₂₅)aryl-S—, perhalo(C₁-C₂₀)alkyl-S—, and a combination thereof.

In another embodiment, said alkylene, cycloalkylene or arylene in said Q and/or T are each independently substituted with one or more substituent groups selected from the group consisting of (C₁-C₂₀)alkyl-SO₂—, phenyl-SO₂—, (C₃-C₂₀)cycloalkyl-SO₂—, (C₁-C₂₀)alkoxy-SO₂—, (C₃-C₂₅)heteroaryl-SO₂—, (C₃-C₂₅)heterocyclic-SO₂—, (C₂-C₂₀)alkenyl-SO₂—, (C₃-C₂₀)cycloalkenyl-SO₂—, (C₂-C₂₀)alkynyl-SO₂—, (C₅-C₂₀)cycloalkynyl-SO₂—, (C₅-C₂₅)aryl-SO₂—, perhalo(C₁-C₂₀)alkyl-SO₂—, and a combination thereof.

In another embodiment, said alkylene, cycloalkylene or arylene in said Q and/or T are each independently substituted with one or more substituent groups selected from the group consisting of H₂N—SO₂—, (C₁-C₂₀)alkyl-NH—SO₂—, phenyl-NH—SO₂—, (C₃-C₂₀)cycloalkyl-NH—SO₂—, (C₁-C₂₀)alkoxy-NH—SO₂—, (C₃-C₂₅)heteroaryl-NH—SO₂—, (C₃-C₂₅)heterocyclic-NH—SO₂—, (C₂-C₂₀)alkenyl-NH—SO₂—, (C₃-C₂₀) cycloalkenyl-NH—SO₂—, (C₂-C₂₀)alkynyl-NH—SO₂—, (C₅-C₂₀)cycloalkynyl-NH—SO₂—, (C₅-C₂₅)aryl-NH—SO₂—, perhalo(C₁-C₂₀)alkyl-NH—SO₂—, salts thereof, and a combination thereof.

In another embodiment, said alkylene, cycloalkylene or arylene in said Q and/or T are each independently substituted with one or more substituent groups selected from the group consisting of {(C₁-C₂₀)alkyl}₂N—SO₂—, {phenyl}₂N—SO₂—, {(C₃-C₂₀)cycloalkyl}₂N—SO₂—, {(C₁-C₂₀)alkoxyl}₂N—SO₂—, {(C₃-C₂₅)heteroaryl}₂N—SO₂—, {(C₃-C₂₅)heterocyclic}₂N—SO₂—, {(C₂-C₂₀)alkenyl}₂N—SO₂—, {(C₂-C₂₀)alkynyl}₂N—SO₂—, {(C₅-C₂₀)cycloalkynyl}₂N—SO₂—, {(C₅-C₂₅)aryl}₂N—SO₂—, {perhalo(C₁-C₂₀)alkyl}₂N—SO₂—, salts thereof, and a combination thereof.

In another embodiment, said alkylene, cycloalkylene or arylene in said Q and/or T are each independently substituted with one or more substituent groups selected from the group consisting of (C₁-C₂₀)alkyl-SO₂—NH—, phenyl-SO₂—NH—, (C₃-C₂₀)cycloalkyl-SO₂—NH—, (C₁-C₂₀)alkoxy-SO₂—NH—, (C₃-C₂₅)heteroaryl-SO₂—NH—, (C₃-C₂₅)heterocyclic-SO₂—NH—, (C₂-C₂₀)alkenyl-SO₂—NH—, (C₃-C₂₀)cycloalkenyl-SO₂—NH—, (C₂-C₂₀)alkynyl-SO₂—NH—, (C₅-C₂₀)cycloalkynyl-SO₂—NH—, (C₅-C₂₅)aryl-SO₂—NH—, perhalo(C₁-C₂₀)alkyl-SO₂—NH—, salts thereof, and a combination thereof.

In another embodiment, said alkylene, cycloalkylene or arylene in said Q and/or T are each independently substituted with one or more substituent groups selected from the group consisting of (C₁-C₂₀)alkyl-NH—, phenyl-NH—, (C₃-C₂₀)cycloalkyl-NH—, (C₁-C₂₀)alkoxy-NH—, (C₃-C₂₅)heteroaryl-NH—, (C₃-C₂₅)heterocyclic-NH—, (C₂-C₂₀)alkenyl-NH—, (C₃-C₂₀)cycloalkenyl-NH—, (C₂-C₂₀)alkynyl-NH—, (C₅-C₂₀)cycloalkynyl-NH—, (C₅-C₂₅)aryl-NH—, perhalo(C₁-C₂₀)alkyl-NH—, salts thereof, and a combination thereof.

In another embodiment, said alkylene, cycloalkylene or arylene in said Q and/or T are each independently substituted with one or more substituent groups selected from the group consisting of {(C₁-C₂₀)alkyl}₂N—, {phenyl}₂N—, {(C₃-C₂₀)cycloalkyl}₂N—, {(C₁-C₂₀)alkoxy}₂N—, {(C₃-C₂₅)heteroaryl}₂N—, {(C₃-C₂₅)heterocyclic}₂N—, {(C₂-C₂₀)alkenyl}₂N—, {(C₃-C₂₀)cycloalkenyl}₂N—, {(C₂-C₂₀)alkynyl}₂N—, {(C₅-C₂₀)cycloalkynyl}₂N—, {(C₅-C₂₅)aryl}₂N—, {perhalo(C₁-C₂₀)alkyl}₂N—, salts thereof, and a combination thereof.

In another embodiment, said alkylene, cycloalkylene or arylene in said Q and/or T are each independently substituted with one or more substituent groups selected from the group consisting of (C₁-C₂₀)alkyl-(C═O)—NH—, phenyl-(C═O)—NH—, (C₃-C₂₀)cycloalkyl-(C═O)—NH—, (C₁-C₂₀)alkoxy-(C═O)—NH—, (C₃-C₂₅)heteroaryl-(C═O)—NH—, (C₃-C₂₅)heterocyclic-(C═O)—NH—, (C₂-C₂₀)alkenyl-(C═O)—NH—, (C₃-C₂₀)cycloalkenyl-(C═O)—NH—, (C₂-C₂₀)alkynyl-(C═O)—NH—, (C₅-C₂₀)cycloalkynyl-(C═O)—NH—, (C₅-C₂₅)aryl-(C═O)—NH—, perhalo(C₁-C₂₀)alkyl-(C═O)—NH—, salts thereof, and a combination thereof.

In another embodiment, said alkylene, cycloalkylene or arylene in said Q and/or T are each independently substituted with one or more substituent groups selected from the group consisting of (C₁-C₂₀)alkyl-(C═O)—{((C₁-C₂₀)alkyl)N}—, phenyl-(C═O)—{((C₁-C₂₀)alkyl)N}—, (C₃-C₂₀)cycloalkyl-(C═O)—{((C₁-C₂₀)alkyl)N}—, (C₁-C₂₀)alkoxy-(C═O)—{((C₁-C₂₀)alkyl)N}—, (C₃-C₂₅)heteroaryl-(C═O)—{((C₁-C₂₀)alkyl)N}—, (C₃-C₂₅)heterocyclic-(C═O)—{((C₁-C₂₀)alkyl)N}—, (C₂-C₂₀)alkenyl-(C═O)—{((C₁-C₂₀)alkyl)N}—, (C₃-C₂₀)cycloalkenyl-(C═O)—{((C₁-C₂₀)alkyl)N}—, (C₂-C₂₀)alkynyl-(C═O)—{((C₁-C₂₀)alkyl)N}—, (C₅-C₂₀)cycloalkynyl-(C═O)—{((C₁-C₂₀)alkyl)N}—, (C₅-C₂₅)aryl-(C═O)—{((C₁-C₂₀)alkyl)N}—, perhalo(C₁-C₂₀)alkyl-(C═O)—{((C₁-C₂₀)alkyl)N}—, salts thereof, and a combination thereof.

In another embodiment, said alkylene, cycloalkylene or arylene in said Q and/or T are each independently substituted with one or more substituent groups selected from the group consisting of phenyl-(C═O)—NH—, phenyl-(C═O)-{(phenyl)N}—, (C₁-C₂₀)alkyl-(C═O)-{(phenyl)N}—, (C₃-C₂₀)cycloalkyl-(C═O)-{(phenyl)N}—, (C₁-C₂₀)alkoxy-(C═O)-{(phenyl)N}—, (C₃-C₂₅)heteroaryl-(C═O)-{(phenyl)N}—, (C₃-C₂₅)heterocyclic-(C═O)-{(phenyl)N}—, (C₂-C₂₀)alkenyl-(C═O)-{(phenyl)N}—, (C₃-C₂₀)cycloalkenyl-(C═O)-{(phenyl)N}—, (C₂-C₂₀)alkynyl-(C═O)-{(phenyl)N}—, (C₅-C₂₀)cycloalkynyl-(C═O)-{(phenyl)N}—, (C₅-C₂₅)aryl-(C═O)-{(phenyl)N}—, perhalo(C₁-C₂₀)alkyl-(C═O)-{(phenyl)N}—, salts thereof, and a combination thereof.

In another embodiment, said alkylene, cycloalkylene or arylene in said Q and/or T are each independently substituted with one or more substituent groups selected from the group consisting of H₂N(C═O)—, (C₁-C₂₀)alkyl-NH—(C═O)—, phenyl-NH—(C═O)—, (C₃-C₂₀)cycloalkyl-NH—(C═O)—, (C₁-C₂₀)alkoxy-NH—(C═O)—, (C₃-C₂₅)heteroaryl-NH—(C═O)—, (C₃-C₂₅)heterocyclic-NH—(C═O)—, (C₂-C₂₀)alkenyl-NH—(C═O)—, (C₃-C₂₀)cycloalkenyl-NH—(C═O)—, (C₂-C₂₀)alkynyl-NH—(C═O)—, (C₅-C₂₀)cycloalkynyl-NH—(C═O)—, (C₅-C₂₅)aryl-NH—(C═O)—, perhalo(C₁-C₂₀)alkyl-NH—(C═O)—, salts thereof, and a combination thereof.

In another embodiment, said alkylene, cycloalkylene or arylene in said Q and/or T are each independently substituted with one or more substituent groups selected from the group consisting of {C₁-C₂₀)alkyl}₂N—(C═O)—, {phenyl}{(C₁-C₂₀)alkyl}N—(C═O)—, {(C₃-C₂₀)cycloalkyl}{(C₁-C₂₀)alkyl}N—(C═O)—, {(C₁-C₂₀)alkoxyl}{(C₁-C₂₀)alkyl}N—(C═O)—,{(C₃-C₂₅)heteroaryl}{(C₁-C₂₀)alkyl}N—(C═O)—, {(C₃-C₂₅)heterocyclic}{(C₁-C₂₀)alkyl}N—(C═O)—, {(C₂-C₂₀)alkenyl}{(C₁-C₂₀)alkyl}N—(C═O)—, {(C₃-C₂₀)cycloalkenyl}{(C₁-C₂₀)alkyl}N—(C═O)—, {(C₂-C₂₀)alkynyl}{(C₁-C₂₀)alkyl}N—(C═O)—, {(C₅-C₂₀)cycloalkynyl}{(C₁-C₂₀)alkyl}N—(C═O)—, {(C₅-C₂₅)aryl}{(C₁-C₂₀)alkyl}N—(C═O)—, {perhalo(C₁-C₂₀)alkyl}{(C₁-C₂₀)alkyl}N—(C═O)—, salts thereof, and a combination thereof.

In another embodiment, said alkylene, cycloalkylene or arylene in said Q and/or T are each independently substituted with one or more substituent groups selected from the group consisting of {phenyl}₂N—(C═O)—, {(C₃-C₂₀)cycloalkyl}{phenyl}N—(C═O)—, {(C₁-C₂₀)alkoxyl}{phenyl}N—(C═O)—, {(C₃-C₂₅)heteroaryl}{phenyl}N—(C═O)—, {(C₃-C₂₅)heterocyclic}{phenyl}N—(C═O)—, {(C₂-C₂₀)alkenyl}{phenyl}N—(C═O)—, {(C₃-C₂₀)cycloalkenyl}{phenyl}N—(C═O)—, {(C₂-C₂₀)alkynyl}{phenyl}N—(C═O)—, {(C₅-C₂₀)cycloalkynyl}{phenyl}N—(C═O)—, {(C₅-C₂₅)aryl}{phenyl}N—(C═O)—, {perhalo(C₁-C₂₀)alkyl}{phenyl}N—(C═O)—, salts thereof, and a combination thereof.

In another embodiment, said alkylene, cycloalkylene or arylene in said Q and/or T are each independently substituted with one or more substituent groups selected from the group consisting of HO—(C═O)—, (C₁-C₂₀)alkyl-(C═O)—, (C₃-C₂₅)heteroaryl-(C═O)—, (C₃-C₂₅)heterocyclic-(C═O)—, (C₂-C₂₀)alkenyl-(C═O)—, (C₃-C₂₀)cycloalkenyl-(C═O)—, (C₂-C₂₀)alkynyl-(C═O)—, (C₅-C₂₅)aryl-(C═O)—, perhalo(C₁-C₂₀)alkyl-(C═O)—, phenyl-(C═O)—, and a combination thereof.

In another embodiment, said alkylene, cycloalkylene or arylene in said Q and/or T are each independently substituted with one or more substituent groups selected from the group consisting of (C₁-C₂₀)alkyl-O—(C═O)—, (C₃-C₂₅)heteroaryl-O—(C═O)—, (C₃-C₂₅)heterocyclic-O—(C═O)—, (C₂-C₂₀)alkenyl-O—(C═O)—, (C₃-C₂₀)-cycloalkenyl-O—(C═O)—, (C₂-C₂₀)alkynyl-O(C═O)—, (C₅-C₂₅)aryl-O(C═O)—, perhalo(C₁-C₂₀)alkyl-O(C═O)—, phenyl-O(C═O)—, and a combination thereof.

In another embodiment, said alkylene, cycloalkylene or arylene in said Q and/or T are each independently substituted with one or more substituent groups selected from the group consisting of (C₁-C₂₀)alkyl-(C═O)—O—, (C₃-C₂₅)heteroaryl-(C═O)—O—, (C₃-C₂₅)heterocyclic-(C═O)—O—, (C₂-C₂₀)alkenyl-(C═O)—O—, (C₃-C₂₀) cycloalkenyl-(C═O)—O—, (C₂-C₂₀)alkynyl-(C═O)—O—, (C₅-C₂₅)aryl-(C═O)—O—, phenyl-(C═O)—O, perhalo(C₁-C₂₀)alkyl-(C═O)—O—, and a combination thereof.

When the Z group or any of its constituent A, Q, T, or CRR groups are substituted, the substituent is preferably a pharmaceutically acceptable or suitable substituent. This type of substituent is intended to mean a chemically and pharmaceutically acceptable functional group (e.g., a moiety that does not negate the pharmaceutical activity of the active compound.)

In one embodiment, the suitable pharmaceutically acceptable substituents include, but are not limited to halo groups, perfluoroalkyl groups, perfluoroalkoxy groups, alkyl groups, alkenyl groups, alkynyl groups, hydroxy groups, oxo groups, mercapto groups, alkylthio groups, alkoxy groups, aryl or heteroaryl groups, aryloxy or heteroaryloxy groups, aralkyl or heteroaralkyl groups, aralkoxy or heteroaralkoxy groups, HO—(C═O)— groups, amino groups, alkyl- and dialkylamino groups, carbamoyl groups, alkylcarbonyl groups, alkoxycarbonyl groups, alkylaminocarbonyl groups, dialkylamino carbonyl groups, arylcarbonyl groups, aryloxycarbonyl groups, alkylsulfonyl groups, arylsulfonyl groups and the like.

As used herein, the term, “alkylene” refers to a diradical alkane species that contains 1, 2, 3, 4, 5, 6, 7, 8, 9, 10, 11, 12, 13, 14, 15, 16, 17, 18, 19 and 20 carbons or any subrange of carbons therebetween. The alkylene may be branched or unbranched, saturated or unsaturated, and substituted or unsubstituted with one or more suitable substituents defined herein, for example with one or more fluoro, chloro, trifluoromethyl, (C₁-C₃)alkoxy, trifluoromethoxy, difluoromethoxy or (C₁-C₃)alkyl. In addition, any carbon atom therein may be optionally replaced with one or more heteroatoms such as nitrogen, oxygen or sulfur or any combination thereof.

As used herein, the term, “cycloalkylene” refers to a diradical cycloalkane species that contains 3, 4, 5, 6, 7, 8, 9, 10, 11, 12, 13, 14, 15, 16, 17, 18, 19 and 20 ring carbons or any subrange of carbons therebetween. The cycloalkylene may be branched or unbranched, saturated or unsaturated, and substituted or unsubstituted with one or more suitable substituents defined herein, for example with one or more fluoro, chloro, trifluoromethyl, (C₁-C₃)alkoxy, trifluoromethoxy, difluoromethoxy or (C₁-C₃)alkyl. In addition, any carbon atom therein may be optionally replaced with one or more heteroatom such as nitrogen, oxygen or sulfur or any combination thereof.

As used herein, the term “arylene” means an aromatic diradical species having 5, 6, 7, 8, 9, 10, 11, 12, 13, 14, 15, 16, 17, 18, 19, 20, 21, 22, 23, 24 and 25 carbons and any subrange of carbons thereof. These may be unsubstituted or substituted with one or more suitable substituents defined herein, for example with one or more fluoro, chloro, trifluoromethyl, (C₁-C₃)alkoxy, trifluoromethoxy, difluoromethoxy or (C₁-C₃)alkyl. In addition, any carbon atom therein may be optionally replaced with one or more heteroatom such as nitrogen, oxygen or sulfur or any combination thereof to form a heteroarylene.

As used herein, the term “alkyl” as well as the alkyl moieties of or within other groups referred to herein (e.g., (C₁-C₂₀)alkyl, (C₁-C₂₀)alkoxy, (C₂-C₂₀)alkenyl, (C₂-C₂₀)alkynyl, and perhalo(C₁-C₂₀)alkyl) include alkyl moieties having 1, 2, 3, 4, 5, 6, 7, 8, 9, 10, 11, 12, 13, 14, 15, 16, 17, 18, 19 and 20 carbons or any subrange of carbons therebetween. They may be linear or branched (such as methyl, ethyl, n-propyl, isopropyl, n-butyl, iso-butyl, secondary-butyl, tertiary-butyl, etc.). They may be saturated or unsaturated as indicated by the “alkenyl” or “alkynyl” terminology. Other than the perhaloalkyl, which are completely substituted by one or more of the same or different halogens, the alkyl groups may be unsubstituted or substituted with one or more suitable substituents defined herein, for example with one or more fluoro, chloro, trifluoromethyl, (C₁-C₃)alkoxy, trifluoromethoxy, difluoromethoxy or (C₁-C₃)alkyl.

As used herein, the term “cycloalkyl” as well as the other moieties having cyclic groups referred to herein (for example (C₃-C₂₀)cycloalkyl, (C₃-C₂₀) cycloalkenyl and (C₅-C₂₀)cycloalkynyl) refers to mono, di, or tri carbocyclic moieties having 3, 4, 5, 6, 7, 8, 9, 10, 11, 12, 13, 14, 15, 16, 17, 18, 19 and 20 ring carbons or any subrange of carbons therebetween. They may be unsubstituted or substituted with one or more suitable substituents defined herein, for example with one or more fluoro, chloro, trifluoromethyl, (C₁-C₃)alkoxy, trifluoromethoxy, difluoromethoxy or (C₁-C₃)alkyl.

As used herein, the terms, “alkenyl,” “alkynyl,” “cycloalkynyl,” and “cycloalkenyl” refer to unsaturated radical species having 1, 2, 3, 4, 5, 6, 7, 8, 9, 10, 11, 12, 13, 14, 15, 16, 17, 18, 19 and 20 carbons (or, for the cyclic species 3, 4, 5, 6, 7, 8, 9, 10, 11, 12, 13, 14, 15, 16, 17, 18, 19 and 20 ring carbons) or any subrange of carbons or ring carbons therebetween. They may be branched or unbranched, and they may be unsubstituted or substituted with one or more suitable substituents defined herein, for example with one or more fluoro, chloro, trifluoromethyl, (C₁-C₃)alkoxy, trifluoromethoxy, difluoromethoxy or (C₁-C₃)alkyl. These groups have one or more than one site of unsaturation, i.e., one or more double or triple bonds. For example, these moieties may have one, two, three, four or more sites of unsaturation. Some nonlimiting examples of these include ethenyl, 1-propenyl, 2-propenyl (allyl), iso-propenyl, 2-methyl-1-propenyl, 1-butenyl, 2-butenyl, ethynyl, 1-propynyl, 2-propynyl, 1-butynyl, and 2-butynyl.

As used herein, the term, “alkoxy” refers to alkyl-O— radical species having 1, 2, 3, 4, 5, 6, 7, 8, 9, 10, 11, 12, 13, 14, 15, 16, 17, 18, 19 and 20 carbons or any subrange of carbons therebetween. They may be unsubstituted or substituted with one or more suitable substituents defined herein, for example with one or more fluoro, chloro, trifluoromethyl, (C₁-C₃)alkoxy, trifluoromethoxy, difluoromethoxy or (C₁-C₃)alkyl.

As used herein, the term “halogen” or “halo” includes fluoro, chloro, bromo or iodo, and any combination thereof.

As used herein, the term “aryl” means aromatic radicals having 5, 6, 7, 8, 9, 10, 11, 12, 13, 14, 15, 16, 17, 18, 19, 20, 21, 22, 23, 24 and 25 carbons and any subrange of carbons thereof. These may be unsubstituted or substituted with one or more suitable substituents defined herein, for example with one or more fluoro, chloro, trifluoromethyl, (C₁-C₃)alkoxy, trifluoromethoxy, difluoromethoxy or (C₁-C₃)alkyl. Nonlimiting examples include phenyl, naphthyl, tetrahydronaphthyl, indanyl and the like.

As used herein, the term “heteroaryl” refers to an aromatic heterocyclic group with at least one heteroatom selected from O, S and N in the ring and having 3, 4, 5, 6, 7, 8, 9, 10, 11, 12, 13, 14, 15, 16, 17, 18, 19, 20, 21, 22, 23, 24 and 25 ring carbons and any subrange of carbons thereof. The heteroatoms may be present either alone or in any combination. The heteroaryl groups may be unsubstituted or substituted with one or more suitable substituents defined herein, for example with one or more fluoro, chloro, trifluoromethyl, (C₁-C₃)alkoxy, trifluoromethoxy, difluoromethoxy or (C₁-C₃)alkyl. One, two, three, four or more heteroatoms may be present. In addition to the heteroatom, the aromatic group may optionally have up to four N atoms in the ring. Nonlimiting examples of heteroaryl groups include pyridyl, pyrazinyl, pyrimidinyl, pyridazinyl, thienyl, furyl, imidazolyl, pyrrolyl, oxazolyl (e.g., 1,3-oxazolyl, 1,2-oxazolyl), thiazolyl (e.g., 1,2-thiazolyl, 1,3-thiazolyl), pyrazolyl, tetrazolyl, triazolyl (e.g., 1,2,3-triazolyl, 1,2,4-triazolyl), oxadiazolyl (e.g., 1,2,3-oxadiazolyl), thiadiazolyl (e.g., 1,3,4-thiadiazolyl), quinolyl, isoquinolyl, benzothienyl, benzofuryl, indolyl, and the like; which are optionally unsubstituted or substituted with one or more suitable substituents defined herein, for example with one or more fluoro, chloro, trifluoromethyl, (C₁-C₃)alkoxy, trifluoromethoxy, difluoromethoxy or (C₁-C₃)alkyl.

The term “heterocyclic” as used herein refers to a cyclic group containing 3, 4, 5, 6, 7, 8, 9, 10, 11, 12, 13, 14, 15, 16, 17, 18, 19, 20, 21, 22, 23, 24 and 25 ring carbons and any subrange of carbons thereof and hetero atoms selected from N, O, S or NR′. Nonlimiting examples include azetidinyl, tetrahydrofuranyl, imidazolidinyl, pyrrolidinyl, piperidinyl, piperazinyl, oxazolidinyl, thiazolidinyl, pyrazolidinyl, thiomorpholinyl, tetrahydrothiazinyl, tetrahydrothiadiazinyl, morpholinyl, oxetanyl, tetrahydrodiazinyl, oxazinyl, oxathiazinyl, indolinyl, isoindolinyl, quinuclidinyl, chromanyl, isochromanyl, benzoxazinyl and the like. Examples of such monocyclic saturated or partially saturated ring systems are tetrahydrofuran-2-yl, tetrahydrofuran-3-yl, imidazolidin-1-yl, imidazolidin-2-yl, imidazolidin-4-yl, pyrrolidin-1-yl, pyrrolidin-2-yl, pyrrolidin-3-yl, piperidin-1-yl, piperidin-2-yl, piperidin-3-yl, piperazin-1-yl, piperazin-2-yl, piperazin-3-yl, 1,3-oxazolidin-3-yl, isothiazolidine, 1,3-thiazolidin-3-yl, 1,2-pyrazolidin-2-yl, 1,3-pyrazolidin-1-yl, thiomorpholinyl, 1,2-tetrahydrothiazin-2-yl, 1,3-tetrahydrothiazin-3-yl, tetrahydrothiadiazinyl, morpholinyl, 1,2-tetrahydrodiazin-2-yl, 1,3-tetrahydrodiazin-1-yl, 1,4-oxazin-2-yl, 1,2,5-oxathiazin-4-yl and the like; which may be unsubstituted or optionally substituted with one or more suitable substituents defined herein, for example with one or more fluoro, chloro, trifluoromethyl, (C₁-C₃)alkoxy, trifluoromethoxy, difluoromethoxy or (C₁-C₃)alkyl. R′ can be any suitable substituent, for example Y as defined herein, or more preferably methyl.

As used herein, the term “halo-substituted alkyl” refers to an alkyl radical as described above substituted with one or more halogens including, but not limited to, chloromethyl, dichloromethyl, fluoromethyl, difluoromethyl, trifluoromethyl, 2,2,2-trichloroethyl, and the like; optionally further substituted with one or more suitable substituents defined herein, for example fluoro, chloro, trifluoromethyl, (C₁-C₃)alkoxy, trifluoromethoxy, difluoromethoxy or (C₁-C₃)alkyl.

As used herein, the term “carbonyl” or “(C═O)” (as used in phrases such as alkylcarbonyl, alkyl-(C═O)— or alkoxycarbonyl) refers to the joinder of the C═O moiety to a second moiety such as an alkyl or amino group (i.e., an amido group). Alkoxycarbonylamino (i.e., alkoxy(C═O)—NH—) refers to an alkyl carbamate group. The carbonyl group is also equivalently defined herein as (C═O). Alkylcarbonylamino refers to groups such as acetamide.

Guanyhydrazones are known compounds and are readily synthesized according to known methods by one skilled in the art. Given the teaching herein, and the knowledge available to one of ordinary skill to which this disclosure is directed, one can readily make and use the subject matter of the full scope of the claims.

In another embodiment, the guanylhydrazone may be combined with one or more acids to form a pharmaceutically acceptable salt. The acids which are used to prepare the pharmaceutically acceptable acid addition salts of the guanylhydrazone compounds are not particularly limited and include those which form non-toxic acid addition salts, i.e., salts containing pharmacologically acceptable anions. Non-limiting examples of such salts include chloride, bromide, iodide, nitrate, sulfate, bisulfate, phosphate, acid phosphate, diphosphate, citrate, acid citrate, tartrate, bitartrate, succinate, fumarate, tosylate, mesylate, gluconate, saccharate, benzoate, methanesulfonate, ethanesulfonate, benzenesulfonate, p-toluenesulfonate, pamoate (i.e., 1,1′-methylene-bis-(2-hydroxy-3-naphthoate)), bicarbonate, edetate, camsylate, carbonate, dihydrochloride, edentate, edisylate, estolate, esylate, gluceptate, glucoheptonate, gluconate, glutamate, glycollylarsanilate, hexylresorcinate, hydrabamine, hydrobromide, hydrochloride, hydroxynaphthoate, isethionate, lactate, L-lactate, L-tartrate, lactobionate, malate, mandelate, methylbromide, methylnitrate, methylsulfate, mucate, napsylate, pantothenate, polygalacturonate, salicylate, stearate, subacetate, succinate, tannate, teoclate, and triethiodide salts. Combinations of salts are possible.

Any ratio of guanylhydrazone:counterion in the salt form, for example, guanylhydrazone:counterion ratios of 10, 9, 8, 7, 6, 5, 4, 3, 2, 1:1, 2, 3, 4, 5, 6, 7, 8, 9, 10 is suitable. The ratio can be expressed as the number of “Ghy” groups:counterions or as the number of ionic guanylhydrazone molecules:counterions as appropriate. In one embodiment, either the guanylhydrazone or the counterion or both may be multivalent, and the ratio is adjusted accordingly such that the salt may adopt a zero or non-zero charge. Mixed salts are possible.

Other non-limiting examples of suitable salts are disclosed in U.S. Pat. Nos. 7,244,765 and 7,291,647, incorporated herein by reference.

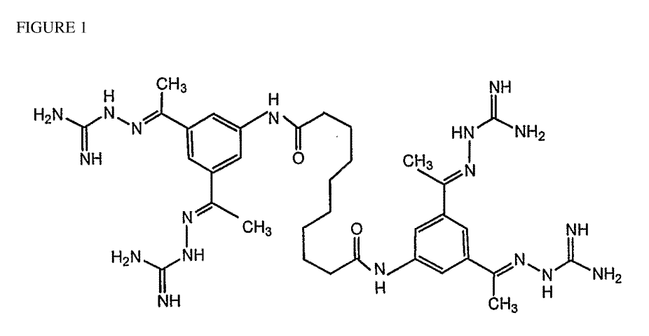

In one embodiment the guanylhydrazone is in the form of a monovalent, divalent, trivalent or tetravalent salt, or in the free base form, or any combination thereof. In another embodiment, the guanylhydrazone is Semapimod (depicted in FIG. 1) or a salt thereof. In another embodiment, the guanylhydrazone is in the form of a tetravalent HCl salt of Semapimod, which salt is known as CNI-1493, and which is obtainable from Cytokine Pharmasciences Inc., King of Prussia, Pa., USA. In another embodiment, the salt may be a tetravalent methanesulfonic acid salt of Semapimod, referred to herein as CPSI-2364, and which is obtainable from Cytokine Pharmasciences Inc., King of Prussia, Pa., USA. Although any guanylhydrazone salt may be used, the mesylate salt of the guanylhydrazone may be particularly suitable as it was found that this form is more suitable for oral application than the HCl salt and more effective at lower concentrations or lower doses.

If reference to a guanylhydrazone is made, this also encompasses any salt form of said guanylhydrazone. As an example, reference to “Semapimod” also encompasses a “salt of Semapimod”. Likewise, whenever reference is made to “a guanylhydrazone”, this is to be read in the meaning of “at least one guanylhydrazone”.

Further, as used herein, the term “subject” refers to a mammal (e.g., any veterinary medicine patient such as a pig, goat, cattle, horse, goat, and the like, any domesticated animal such as a dog or cat and the like), or a human patient.

Given the teachings herein and the knowledge available to one of ordinary skill in the art to which the disclosure relates, one can easily determine a subject either in need of administration of the guanylhydrazone compounds and/or salts thereof or compositions containing same or at risk of or suffering from any of the following: surgical or other manipulation of the intestine, postoperative ileus, inflammation in the muscularis externa (ME) that is mediated by early activation of the p38-MAPK pathway, inflammation after surgical or other manipulation of the intestine, suppression of smooth muscle function, suppression of gastrointestinal motility, ischemia reperfusion injury, ischemia reperfusion injury occurring during small bowel transplantation, inflammation in graft muscularis, graft dysmotility, iatrogenic complications, p38 MAPK (mitogen activated protein kinase) phosphorylation and/or nitric oxide production within the mucosa, submucosa, and/or tunica muscularis, postoperative inflammatory response of the intestine, iatrogenic complications associated with postoperative inflammatory response of the intestine, postoperative inflammatory reaction of the intestine in response to mechanical trauma, increased proinflammatory gene expression and inflammation following intestinal manipulation, increased or delayed gastrointestinal transit, increased or delayed colonic transit, increased or delayed time to first defecation following surgical or other manipulation of the intestine, any combination thereof, and the like.

A “mesylate” as used herein is any salt of methanesulfonic acid (CH₃SO₃H). In the mesylate salts described herein, the mesylate is present as one or more CH₃SO₃⁻ anions.

As appropriate, “pharmaceutically effective amount” or “therapeutically effective amount” or “preventively effective amount” or “prophylactically effective amount” as used herein have their normal meanings, for example, of an amount or dose of at least one guanylhydrazone and/or salt thereof sufficient to decrease, avert, and/or inhibit the progress of one or more of postoperative inflammatory response of the intestine, postoperative ileus, inflammation in the muscularis externa (ME) that is mediated by early activation of the p38-MAPK pathway, inflammation after surgical or other manipulation of the intestine, suppression of smooth muscle function, suppression of gastrointestinal motility, ischemia reperfusion injury, ischemia reperfusion injury occurring during small bowel transplantation, inflammation in graft muscularis, graft dysmotility, iatrogenic complications, p38 MAPK (mitogen activated protein kinase) phosphorylation and/or nitric oxide production within the mucosa, submucosa, and/or tunica muscularis, iatrogenic complications associated with postoperative inflammatory response of the intestine, postoperative inflammatory reaction of the intestine in response to mechanical trauma, increased proinflammatory gene expression and inflammation following intestinal manipulation, increased or delayed gastrointestinal transit, increased or delayed colonic transit, increased or delayed time to first defecation following surgical or other manipulation of the intestine, any combination thereof, and the like, in a subject. Such a decrease in postoperative inflammatory responses can for example, inter alia, be determined by detection of the inhibition of the synthesis of proinflammatory cytokines (e.g. IL-6, MIP 1α and (3, MCP-1 and TNF-α), by inhibition of p38 MAP kinase, or by a decrease in activation of macrophages. In one embodiment, the decrease, aversion, or inhibition can be partial, substantial, or complete. In one embodiment, the decrease, aversion, or inhibition is detectable.

As appropriate, the terms “prevent”, “ameliorate”, and the like as used herein have their normal meanings, for example to decrease, avert, and/or inhibit the progress of one or more of postoperative inflammatory response of the intestine, postoperative ileus, inflammation in the muscularis externa (ME) that is mediated by early activation of the p38-MAPK pathway, inflammation after surgical or other manipulation of the intestine, suppression of smooth muscle function, suppression of gastrointestinal motility, ischemia reperfusion injury, ischemia reperfusion injury occurring during small bowel transplantation, inflammation in graft muscularis, graft dysmotility, iatrogenic complications, p38 MAPK (mitogen activated protein kinase) phosphorylation and/or nitric oxide production within the mucosa, submucosa, and/or tunica muscularis, iatrogenic complications associated with postoperative inflammatory response of the intestine, postoperative inflammatory reaction of the intestine in response to mechanical trauma, increased proinflammatory gene expression and inflammation following intestinal manipulation, increased or delayed gastrointestinal transit, increased or delayed colonic transit, increased or delayed time to first defecation following surgical or other manipulation of the intestine, any combination thereof, and the like, in a subject. In one embodiment, the decrease, aversion, or inhibition can be partial, substantial, or complete. In one embodiment, the decrease, aversion, or inhibition is detectable.

As used herein “prior to” surgery means that the guanylhydrazone and/or salt thereof or pharmaceutical composition thereof is administered to the subject before the surgical procedure is carried out. In one embodiment, the guanylhydrazone or pharmaceutical composition is administered to the subject within 48 hrs before the surgical procedure. This range includes all values and subranges therebetween, including about 0.5, 0.75, 1, 2, 3, 4, 5, 6, 7, 8, 9, 10, 12, 14, 16, 18, 20, 24, 36, and 48 hours prior to surgery, or any combination thereof. In one embodiment, the guanylhydrazone is administered between about 24 and 48 hours prior to surgery. In another embodiment, the guanylhydrazone or pharmaceutical composition is administered between about 12 and 24 hours prior to surgery.

In another embodiment, the guanylhydrazone or pharmaceutical composition is administered between about 8 and 12 hours prior to surgery. In another embodiment, the guanylhydrazone or pharmaceutical composition is administered between about 4 and 8 hours prior to surgery. In another embodiment, the guanylhydrazone or pharmaceutical composition is administered between about 2 and 4 hours prior to surgery. In another embodiment, the guanylhydrazone or pharmaceutical composition is administered between about 1 and 2 hours prior to surgery. In another embodiment, the guanylhydrazone or pharmaceutical composition is administered to the subject between about 30 to 90 minutes before the surgical procedure.

A “pharmaceutically acceptable carrier” as used herein can contain physiologically acceptable compounds that act, for example, to solubilize, disperse, emulsify, dilute, stabilize, or increase the absorption of, or any combination thereof, the active compound. The physiologically acceptable compounds may include, for example, carbohydrates, such as glucose, sucrose or dextrans, antioxidants, such as ascorbic acid or glutathione, chelating agents, buffers, low molecular weight proteins, fetuin, or other stabilizers or excipients. One skilled in the art would know that the choice of a pharmaceutically acceptable carrier, including a physiologically acceptable compound, depends, for example, on the route of administration of the composition. The pharmaceutical composition could be in the form of a liquid, gel, tablet, capsule, etc.

The term “ileus” as used herein refers to a disruption of the normal propulsive gastrointestinal motor activity from non-mechanical mechanisms.

Unless otherwise noted, the term, “intestine” includes any part of the gastrointestinal tract of a subject from the stomach to the anus and any combination thereof. For example, and without limitation, included are the stomach, duodenum, small intestine, jejunum, ileum, colon, cecum, rectum, anus, or any combination thereof, and the like.

One embodiment relates to the use of at least one guanylhydrazone or salt thereof for the preparation of a pharmaceutical composition for the prevention of postoperative intestinal inflammation, postoperative ileus and/or amelioration of ischemia reperfusion injury. Another embodiment relates to a guanylhydrazone (or salt thereof) for the prevention of postoperative intestinal inflammation, postoperative ileus and/or for the amelioration of ischemia reperfusion injuries.

In one embodiment the salt of the guanylhydrazone is a pharmaceutically acceptable salt.

In one embodiment, the guanylhydrazone or salt thereof is an inhibitor of p38 MAPK phosphorylation and/or abrogates nitric oxide production within the gastrointestinal tract. In one embodiment, the nitric oxide production is partially, substantially, or completely abrogated within the mucosa, the submucosa and/or the tunica muscularis by the guanylhydrazone or salt thereof.

In one embodiment, the guanylhydrazone or salt thereof is specific to macrophages. In another embodiment the guanylhydrazone or salt thereof is an inhibitor of p38 MAPK phosphorylation and partially or substantially or completely abrogates nitric oxide production within the tunica muscularis.

In one embodiment the guanylhydrazones or salt thereof specifically inhibit phosphorylation of p38 MAPK in macrophages and/or abrogate nitric oxide production within the tunica muscularis. One of ordinary skill can readily determine whether a given guanylhydrazone is an inhibitor of p38 MAPK phosphorylation specific to macrophages (see for example Examples 1 and 2 below).

In another embodiment, the pharmaceutical composition comprises the at least one guanylhydrazone and at least one pharmaceutically acceptable carrier that is in contact with said at least one salt. Non-limiting examples of pharmaceutically acceptable carriers include, inter alia, carbohydrates, antioxidants, chelating agents, buffers, low molecular weight proteins or other stabilizers or excipients. Combinations are possible.

In another embodiment the surgical procedure is selected from the group consisting of an abdominal surgery, a cardiothoracic surgery, a trauma surgery, an orthopedic surgery, heart surgery, thorax surgery, transplantation, open surgery, minimally invasive surgery, and small bowel transplant. In one embodiment the transplantation procedure is small bowel transplantation. In one embodiment the surgical procedure is a laparotomy (i.e. a surgical procedure involving an incision through the abdominal wall to gain access into the abdominal cavity), a laparoscopy (i.e. minimally invasive surgery, keyhole surgery or pinhole surgery in which operations in the abdomen are performed through small incisions (for example 0.5-1.5 cm)), an open or minimally invasive surgery. In one embodiment the surgery is a small bowel transplant, and the ischemia reperfusion injury is an ischemia reperfusion damage of the transplanted small bowel (graft). In another embodiment the postoperative intestinal inflammation is an inflammation of a graft, for example the inflammation of a transplanted small bowel. In a further embodiment the surgery is a small bowel transplant, and the guanylhydrazone or pharmaceutical composition is administered to both donor and recipient.

The guanylhydrazones, salts thereof, and/or combinations thereof may be administered by any appropriate means, including but not limited to oral; injection (intravenous, intraperitoneal, intramuscular, subcutaneous); by absorption through epithelial or mucocutaneous linings (oral mucosa, rectal and vaginal epithelial linings, nasopharyngial mucosa, intestinal mucosa); rectally, transdermally, topically, intradermally, intragastrally, intracutanly, intravaginally, intravasally, intranasally, intrabuccally, percutanly, sublingually, inhalative, parenteral, or any other means available within the pharmaceutical arts. In one embodiment the guanylhydrazone or pharmaceutical composition is administered using microspheres.

In one embodiment, the guanylhydrazones or pharmaceutical compositions are administered prior to the onset of the inflammatory reaction of the subject. In one embodiment, the administration occurs directly prior to or close to the surgical procedure. However, additional dosing after the onset of the surgical procedure is also contemplated. For example, additional dosing may be carried out during surgical procedures of longer duration, wherein the term “longer duration” refers to a surgical procedure of at least 2 hours.

In one embodiment, the guanylhydrazones or pharmaceutical compositions are administered in a single dose. However, multiple doses may also be administered as appropriate.

In one embodiment, dosage of the guanylhydrazone and/or salt thereof, or pharmaceutical composition, may vary from about 0.001 μg/kg to about 1000 mg/kg. This includes all values and subranges therebetween, including 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, and 1 μg/kg, 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1 2, 3, 4, 5, 6, 7, 8, 9, 10, 11, 12, 13, 14, 15, 16, 17, 18, 19, 20, 21, 22, 23, 24, 25, 50, 75, 100, 200, 300, 400, 500, 600, 700, 800, 900, and 1000 mg/kg, and any combination thereof, of the guanylhydrazone or pharmaceutical composition.

In one embodiment, if administered parenterally, dosages may vary from about 0.001 μg/kg to about 1000 mg/kg of the guanylhydrazone and/or salt thereof, or pharmaceutical composition. In one embodiment, if administered parenterally, dosages may vary from about 0.001 μg/kg to about 500 mg/kg of the guanylhydrazone and/or salt thereof, or pharmaceutical composition. In one embodiment, a dosage for an adult can be, e.g., 10 mg/kg to 500 mg/kg. In another embodiment, the dosage is 10-100 mg/kg. These ranges include all values and subranges therebetween, including 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, and 1 μg/kg, 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1 2, 3, 4, 5, 6, 7, 8, 9, 10, 11, 12, 13, 14, 15, 16, 17, 18, 19, 20, 21, 22, 23, 24, 25, 50, 75, 100, 200, 300, 400, 500, 600, 700, 800, 900, and 1000 mg/kg as appropriate.

In one embodiment, if administered parenterally, dosages may vary from about 0.001 to about 25 mg/kg of the guanylhydrazone and/or salt thereof, or pharmaceutical composition. In another embodiment, a dosage for an adult can be, e.g., 10 to 500 mg/kg. In another embodiment, the dosage is 10-100 mg/kg.

The guanylhydrazones and/or salts thereof or pharmaceutical compositions may be administered orally, for example, in the form of liquids, tablets, capsules, chewable formulations, or the like. In one embodiment, dosages for oral administration may vary from about 0.001 μg/kg to about 1000 mg/kg. This includes all values and subranges therebetween, including 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, and 1 μg/kg, 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1 2, 3, 4, 5, 6, 7, 8, 9, 10, 11, 12, 13, 14, 15, 16, 17, 18, 19, 20, 21, 22, 23, 24, 25, 50, 75, 100, 200, 300, 400, 500, 600, 700, 800, 900, and 1000 mg/kg, and any combination thereof, of the guanylhydrazones and/or salts thereof or pharmaceutical composition. In one embodiment, a dosage for an adult can be, e.g., 10-1000 mg/kg. In another embodiment, the dosage is 10-100 mg/kg.

In one embodiment, if administered orally, for example, in the form of liquids, tablets, capsules, chewable formulations, or the like, dosages for oral administration may vary from about 0.001 to about 25 mg/kg of the guanylhydrazone and/or salt thereof or pharmaceutical composition. In one embodiment, an adult dosage is 10 mg-1000 mg/kg. In another embodiment, the dosage is 10-100 mg/kg.

In one embodiment, the guanylhydrazone and/or salt thereof or pharmaceutical composition dosage may vary from about 0.001 μg/kg to about 10 mg/kg.

In another embodiment, the guanylhydrazone and/or salt thereof or pharmaceutical composition dosage may vary from about 0.001 mg/kg to about 25 mg/kg.

In another embodiment, the guanylhydrazone and/or salt thereof or pharmaceutical composition dosage may vary from about 0.01 μg/kg to about 20 mg/kg.

It will be understood that the exact dose of the pharmaceutical composition may vary depending on the requirements for treatment of individual subjects. The precise dosage, route of administration and regimen will be determined by the attending physician or veterinarian who will, inter alia, consider factors such as body weight, age and specific symptoms.

In one embodiment, the guanylhydrazone compound or salt thereof may be isotopically-labeled, in which one or more atoms are replaced by an atom having an atomic mass or mass number different from the atomic mass or mass number usually found in nature. Non-limiting examples of suitable isotopes include isotopes of hydrogen, carbon, nitrogen, oxygen, phosphorous, fluorine and chlorine, such as ²H, ³H, ¹³C, ¹⁴C, ¹⁵N, ¹⁸O, ¹⁷O, ³¹P, ³²P, ³⁵S, ¹⁸F, and ³⁶Cl, respectively. The guanylhydrazone compounds, prodrugs thereof, and pharmaceutically acceptable salts of said compounds or of said prodrugs which contain the aforementioned isotopes and/or other isotopes of other atoms are possible. The isotopically-labeled compounds, for example those into which radioactive isotopes such as ³H and ¹⁴C are incorporated, might be useful in drug and/or substrate tissue distribution assays. Tritiated, i.e., ³H, and carbon-14, i.e., ¹⁴C, isotopes may be suitable in view of ease of preparation and detectability. Substitution with heavier isotopes such as deuterium, i.e., ²H, may afford therapeutic advantages resulting from greater metabolic stability, for example increased in vivo half-life or reduced dosage requirements. Isotopically labeled compounds may readily be prepared by substituting a readily available isotopically labeled reagent for a non-isotopically labeled reagent during synthesis or salt formation.

The guanylhydrazone compounds and salts thereof may exist in several tautomeric forms, and geometric isomers and mixtures thereof. Tautomers exist as mixtures of tautomers in solution. It may be the case that in solid form, one tautomer predominates. Absent evidence to the contrary, all such tautomeric forms are included within the scope of the claims, even though only one may be mentioned.

The guanylhydrazones and salts thereof may be present as atropisomers. Atropisomers can be separated into rotationally restricted isomers. For example, the compounds may contain olefin-like double bonds. When such bonds are present, the compounds may exist as cis and trans configurations and as mixtures thereof, and all are contemplated within the scope of the claims.

In one embodiment, a pharmaceutical composition is provided comprising one or more pharmaceutically acceptable salts and one or more a pharmaceutically acceptable carrier, excipient, adjuvant and/or diluents, in addition to the guanylhydrazone compound and/or salt thereof.

Other embodiments relate to methods of making and using the salts, for example wherein the salt is used to assay or test the guanylhydrazone compound.

The salts may be suitably prepared according to known methods, for example, by contacting the free base form of the guanylhydrazone containing compound with a sufficient amount of the desired acid to produce a salt in the conventional manner.

The free base forms may be regenerated by treating the salt with a suitable dilute aqueous base solution such as dilute aqueous sodium hydroxide, potassium carbonate, ammonia, sodium bicarbonate, or combination thereof.

In one embodiment, the guanylhydrazone compounds, salts thereof, or combination thereof may be administered in combination with one or more substantially nontoxic pharmaceutically acceptable carriers, excipients, adjuvants or diluents. The compositions may be prepared in any conventional solid or liquid carrier or diluent and optionally any conventional pharmaceutically-made adjuvant at suitable dosage level in a known way. The preparations may be in administrable form which is suitable for oral application. These administrable forms, for example, include pills, tablets, film tablets, coated tablets, capsules, powders and deposits.

The pharmaceutically acceptable carrier may be suitably selected with respect to the intended form of administration, i.e. oral tablets, capsules (either solid-filled, semi-solid filled or liquid filled), powders for constitution, oral gels, elixirs, dispersible granules, syrups, suspensions, and the like, and consistent with conventional pharmaceutical practices. For example, for oral administration in the form of tablets or capsules, the salt may be combined with any oral nontoxic pharmaceutically acceptable inert carrier, such as lactose, starch, sucrose, cellulose, magnesium stearate, dicalcium phosphate, calcium sulfate, talc, mannitol, ethyl alcohol (liquid forms) and the like. Moreover, when desired or needed, suitable binders, lubricants, disintegrating agents and coloring agents may also be incorporated in the mixture. Powders and tablets may be comprised of from about 5 to about 95 percent by weight of the inventive compound, salt thereof, or a mixture of compound and salt, which range includes all values and subranges therebetween, including 10, 15, 20, 25, 30, 35, 40, 45, 50, 55, 60, 65, 70, 75, 80, 85, and 90% by weight.

In one embodiment, the guanylhydrazones, salts thereof, or combinations thereof may be formulated in sustained release form to provide the rate controlled release of any one or more of the components or active ingredients to optimize the therapeutic effects, i.e. antihistaminic activity and the like. Non-limiting examples of dosage forms for sustained release include layered tablets containing layers of various disintegration rates or controlled release polymeric matrices impregnated with the active components and shaped in tablet form or capsules containing such impregnated or encapsulated porous polymeric matrices.

Liquid form preparations may include solutions, suspensions and emulsions, or combinations thereof. Nonlimiting examples include water, ethanol, ethanolic, water-ethanol or water-propylene glycol solutions for parenteral injections or addition of sweeteners and opacifiers for oral solutions, suspensions and emulsions. Liquid form preparations may also include solutions for intranasal or other administration.

Aerosol preparations suitable for inhalation may include solutions and solids in powder form, which may be in combination with a pharmaceutically acceptable carrier such as inert compressed gas, e.g. nitrogen.

For preparing suppositories, for example, a low melting wax such as a mixture of fatty acid glycerides such as cocoa butter is first melted, and the active ingredient may be dispersed homogeneously therein by stirring or similar mixing. The molten homogeneous mixture may then be poured into convenient sized molds, allowed to cool and thereby solidify.

Also included are solid form preparations which are intended to be converted, shortly before use, to liquid form preparations for either oral or parenteral administration. Examples of such liquid forms include solutions, suspensions and emulsions.

In one embodiment, the guanylhydrazones, salts thereof, or combinations thereof may be deliverable transdermally. The transdermal compositions may take the form of creams, lotions, aerosols and/or emulsions and can be included in a transdermal patch of the matrix or reservoir type as are conventional in the art for this purpose.

The term capsule refers to a special container or enclosure made for example of methyl cellulose, polyvinyl alcohols, or denatured gelatins or starch for holding or containing compositions comprising the active ingredients. Hard shell capsules are typically made of blends of relatively high gel strength bone and pork skin gelatins. The capsule itself may contain small amounts of dyes, opaquing agents, plasticizers and preservatives.

Tablet means compressed or molded solid dosage form containing the active ingredients with suitable diluents. The tablet can be prepared by compression of mixtures or granulations obtained by wet granulation, dry granulation or by compaction well known to a person skilled in the art.

Oral gels refers to the active ingredients dispersed or solubilized in a hydrophillic semi-solid matrix.

Powders for constitution refers to powder blends containing the active ingredients and suitable diluents which can be suspended in water or juices.

Diluents may include substances that may make up the major portion of the composition or dosage form. Non-limiting examples of diluents include sugars such as lactose, sucrose, mannitol and sorbitol, starches derived from wheat, corn rice and potato, and celluloses such as microcrystalline cellulose. If present, the amount of diluent in the composition can range from about 5 to about 95% by weight of the total composition.

Disintegrants may be added to the composition as appropriate to help it break apart (disintegrate) and release the medicaments. Non-limiting examples of disintegrants include starches, “cold water soluble” modified starches such as sodium carboxymethyl starch, natural and synthetic gums such as locust bean, karaya, guar, tragacanth and agar, cellulose derivatives such as methylcellulose and sodium carboxymethylcellulose, microcrystalline celluloses and cross-linked microcrystalline celluloses such as sodium croscarmellose, alginates such as alginic acid and sodium alginate, clays such as bentonites, and effervescent mixtures. If present, the amount of disintegrant in the composition can range from about 2 to about 20% by weight of the composition.

Binders, which are substances that bind or “glue” powders together and make them cohesive by forming granules, thus serving as the “adhesive” may be used in the formulation. Binders add cohesive strength already available in the diluent or bulking agent. Non-limiting examples of binders include sugars such as sucrose, starches derived from wheat, corn rice and potato; natural gums such as acacia, gelatin and tragacanth; derivatives of seaweed such as alginic acid, sodium alginate and ammonium calcium alginate; cellulosic materials such as methylcellulose and sodium carboxymethylcellulose and hydroxypropyl-methylcellulose; polyvinylpyrrolidone; and inorganics such as magnesium aluminum silicate. If present, the amount of binder in the composition can range from about 2 to about 20% by weight of the composition.

Lubricant, which refers to a substance added to the dosage form to enable the tablet, granules, etc. after it has been compressed, to release from the mold or die by reducing friction or wear, may be used. Non-limiting examples of lubricants include metallic stearates such as magnesium stearate, calcium stearate or potassium stearate; stearic acid; high melting point waxes; and water soluble lubricants such as sodium chloride, sodium benzoate, sodium acetate, sodium oleate, polyethylene glycols and d,l-leucine. If desired, the amount of lubricant in the composition can range from about 0.2 to about 5% by weight of the composition.

Glidents, which are materials that prevent caking and improve the flow characteristics of granulations, so that flow is smooth and uniform, may be used. Non-limiting examples include silicon dioxide and talc. If present, the amount of glident in the composition can range from about 0.1% to about 5% by weight of the total composition.

Coloring agents, which provide coloration to the composition or the dosage form, may be used. Non-limiting examples of such excipients can include food grade dyes and food grade dyes adsorbed onto a suitable adsorbent such as clay or aluminum oxide. If present, the amount of the coloring agent can vary from about 0.1 to about 5% by weight of the composition.

The guanylhydrazones, salts thereof, or combinations thereof, or pharmaceutical composition may exist in any convenient crystalline, semicrystalline, or amorphous form. These may be achieved via typical crystallization routes including vacuum crystallization or spray drying. Depending on the solublity desired, the amorphous form obtained by, e.g., spray-drying may be advantageous. The spray drying may be carried out from aqueous, ethanolic, organic, or mixed aqueous ethanolic solutions of the salt or a mixture of the salt and the free base compound. The compound and/or salt may exist in a form comprising one or more waters of hydration.

Other techniques for formulation and administration may be found in “Remington's Pharmaceutical Sciences” Mack Publishing Co., Easton Pa., the entire contents of which are hereby incorporated by reference. In one embodiment, the guanylhydrazone, salt thereof, or combination thereof is in the form of a pharmaceutical composition, which may be a solution of the compound in a suitable liquid pharmaceutical carrier or any other formulation such as tablets, pills, film tablets, coated tablets, dragees, capsules, powders and deposits, gels, syrups, slurries, suspensions, emulsions, and the like.

Another embodiment relates to a use of at least one guanylhydrazone, salt thereof, or combination thereof for the preparation of a pharmaceutical composition for the prevention of postoperative intestinal inflammation, postoperative ileus and/or amelioration of ischemia reperfusion injuries.

Another embodiment relates to a use of at least one guanylhydrazone, salt thereof, or combination thereof for the preparation of a pharmaceutical composition for the prevention of postoperative intestinal inflammation, postoperative ileus and/or amelioration of ischemia reperfusion injuries wherein the guanylhydrazone is an inhibitor of p38 MAP kinase phosphorylation and/or abrogates nitric oxide production within the gastrointestinal tract.

Another embodiment relates to a use of at least one guanylhydrazone, salt thereof, or combination thereof for the preparation of a pharmaceutical composition for the prevention of postoperative intestinal inflammation, postoperative ileus and/or amelioration of ischemia reperfusion injuries, wherein nitric oxide production is abrogated within the mucosa, the submucosa and/or the tunica muscularis.

Another embodiment relates to a use of at least one guanylhydrazone, salt thereof, or combination thereof for the preparation of a pharmaceutical composition for the prevention of postoperative intestinal inflammation, postoperative ileus and/or amelioration of ischemia reperfusion injuries, wherein the at least one guanylhydrazone is CPSI-2364 or CNI-1493.

Another embodiment relates to a use of at least one guanylhydrazone, salt thereof, or combination thereof for the preparation of a pharmaceutical composition for the prevention of postoperative intestinal inflammation, postoperative ileus and/or amelioration of ischemia reperfusion injuries, wherein the pharmaceutical composition is administered at least once prior to a surgical procedure.

Another embodiment relates to a use of at least one guanylhydrazone, salt thereof, or combination thereof for the preparation of a pharmaceutical composition for the prevention of postoperative intestinal inflammation, postoperative ileus and/or amelioration of ischemia reperfusion injuries, wherein the pharmaceutical composition is administered at least once prior to a surgical procedure, and wherein the surgical procedure is selected from the group including abdominal surgery, cardiothoracic surgery, trauma and orthopedic surgery, heart surgery, thorax surgery, transplantation and small bowel transplant.

Another embodiment relates to a use of at least one guanylhydrazone, salt thereof, or combination thereof for the preparation of a pharmaceutical composition for the prevention of postoperative intestinal inflammation, postoperative ileus and/or amelioration of ischemia reperfusion injuries, wherein the pharmaceutical composition is administered at least once prior to a surgical procedure, and wherein the surgical procedure, wherein the surgical procedure is an open or minimally invasive surgery.

Another embodiment relates to a method of preventing postoperative intestinal inflammation, postoperative ileus and/or ameliorating ischemia reperfusion injuries in a subject, the method comprising administering to said subject a therapeutically effective amount of at least one guanylhydrazone, salt thereof, or combination thereof, wherein the administration is carried out up to 48 hours prior to surgery.

Another embodiment relates to a method for inhibiting macrophage chemoattractant protein −1 (MCP-1) comprising administering to a subject in need thereof an MCP-1 inhibiting effective amount of a guanylhydrazone or salt thereof.

### EXAMPLES

### Material and methods

POI Model

Animals

Male C57BL/6J mice (−25 g bodyweight) were obtained from Harlan-Winkelmann (Borchen, Germany). CSF-1 mutant op−/− mice (strain B6C3Fe a/a-Csf1op) and populations of unknown heterozygous or wildtype genotype (op+/?) of mixed gender were used at an age of 6 weeks and bred as described before (Wehner S, Behrendt F F, Lyutenski B N et al. Inhibition of macrophage function prevents intestinal inflammation and postoperative ileus in rodents. Gut 2007; 56:176-185). op−/− mice were phenotyped by the absence of incisors at day P10. All experiments were performed in accordance with the federal law regarding the protection of animals. The principles of laboratory animal care were followed. Animals were maintained on a 12-hour light/dark cycle and provided with commercially available rodent chow and tap water ad libitum.

Operative Procedure

The small bowel of the animals was subjected to a standardized, surgical manipulation (IM) as described previously (Kalff J C, Schraut W H, Simmons R L et al. Surgical manipulation of the gut elicits an intestinal muscularis inflammatory response resulting in postsurgical ileus. Ann Surg 1998; 228:652-663; Wehner S, Schwarz N T, Hundsdoerfer R et al. Induction of IL-6 within the rodent intestinal muscularis after intestinal surgical stress. Surgery 2005; 137:436-446). In brief, after anesthesia a midline abdominal incision was made into the peritoneal cavity. The entire small bowel was eventuated and manipulated with standardized moderate intensity using two moist cotton applicators as described previously. After IM, the laparotomy was closed by one layer of continuous suture, except when probe sampling was conducted within 20 minutes after surgery. These animals were held in anesthesia and the abdomen was closed by a clamp and protected with sterile gauze. All other animals recovered rapidly from the bowel manipulation procedure. Control animals exclusively underwent laparotomy without eventration and IM (sham operation).

Experimental Groups

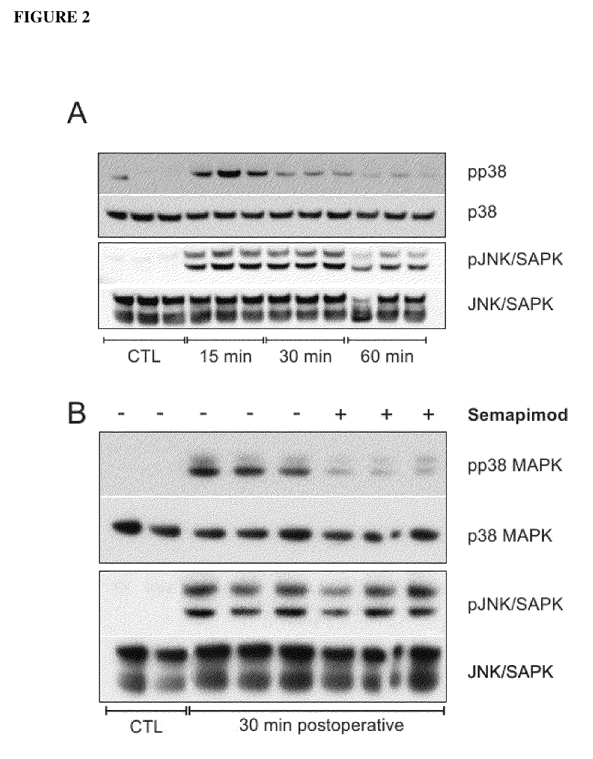

Animals within examples referring to FIGS. 2-9 were treated 90 minutes before operation with 0 mg/kg (placebo group), or 5 mg/kg bodyweight CNI-1493. In further experiments in rats or mice 0.1, 1.0 or 10 mg/kg CPSI-2364 were administered 90 minutes before surgical procedure via oral route. Oral (p.o.) administration was performed via a gastric tube. CNI-1493 and CPSI-2364 were dissolved in 2.5% mannitol. Placebo and CNI-1493 or CPSI-2364 groups were subdivided in two further groups. One was sham operated (laparotomy without IM) while the other underwent laparotomy followed by IM.

Animals were sacrificed after indicated periods and small bowel muscle specimen were prepared for experiments as described with the exception of gastrointestinal and colonic transit studies. Transit studies were performed in vivo 24 hours after operation.

To investigate the effect of CPSI-2364 on postoperative small bowel ischemia/reperfusion injury and muscular function, CPSI-2364 (1 mg/kg bodyweight, solved in 2.5% mannitol) or vehicle (2.5% mannitol) were administered intravenously once to the donor and the recipient 90 minutes before the beginning of the organ retrieval operation as well as the transplantation procedure that was described above. After donor organ recovery, organ was stored for 5 hours in cold UW solution (cold ischemia time) in every group. Control animals were native rats that did not undergo any operative procedure. Small bowel grafts were harvested 3 and 18 hours after reperfusion for further analysis.

p38-MAPK and JNK/SAPK Phosphorylation After Intestinal Manipulation

p38-MAPK and JNK/SAPK phosphorylation were analyzed in C57BL/6J mice 15, 30 and 60 minutes after IM and in unoperated controls. Furthermore, placebo and CNI-1493 (5 mg/kg) i.v. pretreated groups were compared for phosphorylation levels 30 minutes after IM. Therefore, snap frozen ME preparations (30-50 mg) were homogenized in chilled PBS containing 2 mM EDTA/EGTA. Equal volumes of 2×RIPA lysis buffer (100 mM Tris HCl pH 8.0, 300 mM NaCl, 2% NP-40, 1% sodium deoxycholate, 0.2% SDS) containing 2 mM sodium orthovanadate, 2 mM f3-glycerolphosphate and protease inhibitor cocktail (# P8340; Sigma-Aldrich, Taufkirchen, Germany) was added to the PBS-homogenate. Tissue samples were lysed on ice and sonicated twice (Sonopuls U W 2070, Bendelin, Berlin, Germany). Finally, the sonicated samples were centrifuged at 13.000×g for 10 minutes, and total protein concentration was determined with Pierce BCA protein assay (Perbio, Bonn, Germany). Equal volumes were separated in 4-12% Bis-Tris NuPAGE® gels. PAGE was performed with MES running buffer (Invitrogen, Karlsruhe, Germany) for 45 minutes at 200V.

After PAGE, proteins were blotted on Immobilon-P 0.2 μM PVDF membranes. Blotting was performed in an X-Cell II blotting chamber for 1 hour at 2.5 mA/cm2. PVDF membranes were washed with tris-buffered saline containing 0.1% Tween 20 (TBST) and blocked with 5% skim milk/TBST for 1 hour. Primary polyclonal antibodies against phospho-p38-MAPK(Thr180/Tyr182), p38-MAPK, phospho-JNK/SAPK and JNK/SAPK(Thr183/185) were diluted 1:1000 in 5% BSA w/v in TBST and incubated overnight at 4° C. After washing membranes 3 times with TBST, secondary antibody (anti rabbit-HRP, 1:2000) was incubated for 1 h. After three final washes with TBST, membranes were incubated for 5 minutes with Super Signal West Pico substrate (Perbio, Bonn, Germany). Chemiluminscence signals were detected with a LAS 4000 system (Fujifilm, Duesseldorf, Germany).

p38-MAPK Activation in CSF-1 Mutant op−/− Mice

p38-MAPK phosphorylation was evaluated in unoperated homozygous colony stimulating factor-1 mutant mice (op−/−) mice, or in op−/− and op+/? mice (a mixed population of unknown heterozygous +/− or homozygous +/+ wildtype mice) 20 minutes after IM. Animals were treated with placebo i.v. 90 minutes before IM. Additionally, CNI-1493 (5 mg/kg) pretreated op−/− mice were analyzed. In this experiment, p38-MAPK phosphorylation was quantified by the Pathscan-Phospho-p38α-ELISA following manufacturer's instructions. Phospho-p38 content of each probe was determined in duplicate from lysates with 6.25 μg total protein content.

Histochemistry

Specimens for histochemical analysis were prepared 24 h after operation and performed on whole mounts of the distal jejunum, as described before in detail (n=5-7 each group) 9. In brief, jejunal segments were opened, immersed in chilled Krebs-Ringer buffer (KRB) and fixed in 100% ethanol for 10 minutes. After washing with KRB, mucosa and submucosa were stripped off and ME whole mounts were used for detection of myeloperoxidase (MPO) positive cells (neutrophils). Thereto, freshly prepared whole mounts were stained with Hanker-Yates reagent as described previously (Wehner S, Schwarz N T, Hundsdoerfer R et al. Induction of IL-6 within the rodent intestinal muscularis after intestinal surgical stress. Surgery 2005; 137:436-446). MPO+-cells were counted under a microscope (TE-2000, Nikon, Duesseldorf, Germany) in 5 randomly chosen areas in each specimen.

Real-Time RT-PCR

Proinflammatory gene expression was analyzed in placebo and CNI-1493 (5 mg/kg) treated animals at 1, 3, 6 and 24 hours after operation; n=5-7 each group. To verify and demonstrate the induction of proinflammatory gene expression, a placebo treated sham operated group was supplemented. Total RNA was analyzed in ME specimen that was prepared as described before (Wehner S, Behrendt F F, Lyutenski B N et al. Inhibition of macrophage function prevents intestinal inflammation and postoperative ileus in rodents. Gut 2007; 56:176-185). Total RNA extraction was performed using the NucleoSpin®-RNA II kit (Macherey-Nagel, Dueren, Germany) that includes a DNAse-I digestion step to avoid contamination of the RNA by residual genomic DNA. For cDNA synthesis 1000 ng RNA were transcribed. Expression of mRNA was quantified in triplicate by a RT-PCR with gene expression assays for CC12 (MCP-1; #Mm00441242_m1), CCL3 (MIP-1α #Mm00441258_m1), IL-6 (Assay ID #Mm00446190_ml), TNF-α (#Mm00443258_m1) and ICAM-1 (#Mm00516023_m1). The PCR reaction was performed in Universal PCR Mastermix by amplification of 10 ng cDNA for 40 cycles (95° C.×15 sec, 60° C.×1 min) on an AbiPrism 7900HT. Data quantification was performed by the ΔΔC_{T} method. All reagents were obtained from Applied Biosystems, Darmstadt, Germany.

Determination of Nitrite and NO Production

Animals underwent IM after intravenous CNI-1493 (5 mg/kg) or oral CPSI-2364 (1 mg/kg or 0.1 mg/kg) administration 90 minutes before surgery. Control animals received CNI-1493 (5 mg/kg) intravenously or CPSI-2364 orally (1 mg/kg or 0.1 mg/kg), but were not operated.

After preoperative i.v. or oral drug or placebo administration animals underwent IM. Control animals received CNI-1493 or placebo i.v. or orally but were not operated.

Total ME from small intestine was isolated 24 h after operation as described above and then cut into small pieces and washed for 30 minutes in PBS containing a penicillin/streptomycin (200U/200 μg) mixture. Aliquots of ˜50 mg were incubated in 1 mL of DMEM at 37° C. and 5% CO₂ for further 24 hours and tissue culture supernatants were frozen in liquid nitrogen. The muscle tissue was blotted dry, and the exact weight was measured. To quantify the generation of NO from intestinal smooth muscle preparations, nitrite production was measured by the Griess reaction as described previously (Kalff J C, Schraut W H, Billiar T R et al. Role of inducible nitric oxide synthase in postoperative intestinal smooth muscle dysfunction in rodents. Gastroenterology 2000; 118:316-327). In brief, supernatant from the tissue culture was mixed with an equal volume of Griess reagent (0.1% N-(1-napthyl)ethyl-enediamine dihydrochloride and 1% sulfanilamide in 6% phosphoric acid; 1:1) and incubated for 10 minutes. The absorbance at 550 nm was measured with a microplate reader and compared with standard dilutions of sodium nitrite. Total nitrite produced was normalized to 1 g of muscle tissue.

Functional Studies

Jejunal smooth muscle activity was measured as previously described (Kalff J C, Schraut W H, Simmons R L et al. Surgical manipulation of the gut elicits an intestinal muscularis inflammatory response resulting in postsurgical ileus. Ann Surg 1998; 228:652-663). Briefly, after preparation, mucosa-free circular ME strips were equilibrated in KRB perfused organ chambers at 37° C. for 1 hour. One end of each strip was tied to a fixed post and the other attached to an isometric force transducer (ADI, Heidelberg, Germany) connected to the bridge amplifier and powerlab system (ADI). Dose-response curves of muscle contraction were generated by exposing the muscle strips to increasing concentrations of the muscarinic agonist bethanechol (0.1-300 μmol/L) for 10 minutes, followed by a wash-period (KRB) of 10 minutes. The contractile response was analyzed with the ADI Chart© software and the contractions were calculated as grams per square millimeter per second by conversion of the weight and length of the strip to square millimeters of tissue.

Gastrointestinal transit was measured 23.5 hours postoperatively by evaluating the intestinal location of a fluorochrome labeled dextran (FITC—dextran, 70.000 MW, Molecular Probes, Netherlands) (n=10 each). Thereto, 22 hours after the bowel manipulation, animals were lightly anesthetized and given FITC-dextran (200 μl of 6.25 mg/ml stock solution) via a gastric tube into the stomach. Mice were killed 90 minutes after administration, the entire gastrointestinal tract was divided into 15 segments, opened and FITC-dextran was washed out by PBS. Fluorescence of segment washes was read at 494 nm/521 nm wavelength in fluorescence reader (Tecan, Crailsheim, Germany). The data were expressed as the percentage of activity per segment. Gastrointestinal transit was calculated as the geometric center (GC) of distribution of fluorescence marker using this formula:

GC=Σ(% of total fluorescent signal per segment*segment number)/100.

Colonic transit measurement was performed 24 h hours after operation by inserting a 2 mm glass ball with a metal rod 3 cm into the colon. Before insertion colonic patency was ensured by exclusively inserting the rod 3 cm into the colon. Mice were weakly anesthetized with isofluran for the whole procedure and wake up within 40 seconds after glass ball insertion. Colonic transit time was calculated as the period between insertion and excretion of the ball.

Anastomotic Wound Healing

C57BL/6J mice were treated with 0 mg/kg (placebo) or 5 mg/kg CNI-1493 90 minutes before colonic transection followed by an anastomosis with 8-10 interrupted polypropylene 8.0 sutures. On postoperative days (POD) 2, 5 and 10 anastomotic tissue was investigated for disturbances of wound healing by measurement of hydroxyproline content and bursting pressure.

Hydroxyproline content was determined from an 1 cm perianastomotic region as described before (Woessner J. F., Jr. The determination of hydroxyproline in tissue and protein samples containing small proportions of this imino acid. Arch Biochem Biophys 1961; 93:440-447). Results were normalized as micrograms of hydroxyproline per gram tissue.

Anastomotic bursting pressure (ABP) was measured directly after sacrifice and sampling a 3 cm colonic segment including the anastomotic site. The anastomotic specimen was cleared from feces and ligated twice at the distal end using a 6-0 polyglactin (Vicryl™, Ethicon, Belgium) suture. An 18 G arterial catheter (Vygon, France) was inserted intraluminally within the specimen at the proximal end and two stay sutures were tied to prevent leakage. The catheter was connected to an infusion pump and KHB was infused at a constant rate of 1.65 ml/h. The intraluminal pressure (mmHg) was measured and recorded using a pressure transducer with an amplifier and recorded by the Biopac A/D systems (Biopac Systems, Goleta, USA). ABP was indicated as a sudden loss of pressure and defined as the maximum intraluminal pressure prior to leakage.

Drugs and Solutions

A standard KRB solution was used with the following constituents (μM): Na⁺, 137.4; K⁺, 5.9; Ca²⁺, 2.5; Mg⁺, 1.2; Cl⁻, 134; HCO₃⁻, 15.5; H₂PO₄⁻, 1.2; and glucose, 11.5. PBS was purchased from Lonza (Verviers, Belgium). All other chemicals used for this study (if not separately mentioned) were purchased from Sigma-Aldrich (Taufkirchen, Germany). CNI-1493 was tested. All antibodies and Pathscan-ELISA were purchased from NEB (Cell

Signaling Technology, Frankfurt, Germany)

Data Analysis

Statistical analysis was performed using two-way ANOVA followed by a Bonferroni posttest. Significance level were p<0.05 (*), p<0.01 (**) and p<0.001 (***).

Small Bowel Transplant Model

Animals

Inbred male Lewis rats weighing 180-200 g were obtained from Charles River GmbH (Sulzfeld/Germany). All experiments were performed in accordance with the federal law regarding the protection of animals. The principles of laboratory animal care were followed. The animals were maintained on a 12-hour light/dark cycle and provided with commercially available chow (S sniff, Soest, Germany) and tap water ad libitum.

Experimental Procedure: Small Intestinal Transplantation

Syngeneic orthotopic small intestinal transplantation was performed in Lewis rats as previously described (Schwarz N T, Nakao A, Nalesnik M A, Kalff J C, Murase N, Bauer A J. Protective effects of ex vivo graft radiation and tacrolimus on syngeneic transplanted rat small bowel motility. Surgery 2002; 131: 413-423). Briefly, the small intestine of the donor was isolated from the ligament of Treitz to the ileocecal valve with its vascular pedicle consisting of the superior mesenteric artery with a piece of aorta and the superior mesenteric vein. Then the graft vascular bed was perfused with chilled UW solution (University of Wisconcin) and the intestinal lumen was irrigated with 50 ml cold 0.9% NaCl solution containing 10.0001.E. neomycin sulfate (Uro Nebacetin N, Nycomed, Germany). The graft was stored in cold UW solution during the preparation of the recipient. An end-to-side anastomosis between the graft aorta and the recipient infra-renal aorta, and between the graft vein and recipient vena cava, was performed. Approximately 80% of the recipient intestine was removed and enteric continuity was restored by proximal and distal end-to-end intestinal anastomoses.

Experimental Groups

To investigate the effect of CPSI-2364 on postoperative small bowel ischemia/reperfusion injury and muscular function, CPSI-2364 (1 mg/kg bodyweight, solved in 2.5% mannitol) or vehicle (2.5% mannitol) were administered intravenously once to the donor and the recipient 90 minutes before the beginning of the organ retrieval operation as well as the transplantation procedure that was described above. After donor organ recovery, organ was stored for 5 hours in cold UW solution (cold ischemia time) in every group. Control animals were native rats that did not undergo any operative procedure. Small bowel grafts were harvested 3 and 18 hours after reperfusion for further analysis.

Determination of Intestinal Injury

Histological changes and intestinal injury were graded by determination of the Park score 3 and 18 hours after reperfusion. The Parks' system scores mucosal injury in intestinal villi and in crypts separately from grade 0 to 8, according to the degree or intestinal wall architecture destruction. Probes were analyzed in a blinded way.

Quantification of Leukocyte Infiltration and Monocyte/Macrophage Infiltration

Specimens for histochemical analysis were prepared 18 h after operation and performed on whole mounts of the distal jejunum (n=8 each group). Whole mount preparation were fixed with 100% ethanol for 10 minutes or 4% PFA for 30 min at room temperature.

For detection of myeloperoxidase positive neutrophils (MPO+), freshly prepared whole mounts were stained with Hanker-Yates reagent as described previously (Wehner S, Schwarz N T, Hundsdoerfer R et al. Induction of IL-6 within the rodent intestinal muscularis after intestinal surgical stress. Surgery 2005; 137: 436-446). Infiltrated monocyte and immature macrophages were stained with primary anti CD68 antibody (Serotec clone ED1, Germany, 1:200 at 4° C. over night) and secondary donkey anti mouse-Cy3 (Dianova, Heidelberg, Germany, 1:200).

ED1 and MPO+-cells were counted under a microscope (TE-2000, Nikon, Duesseldorf, Germany) in 5 randomly chosen areas in each specimen.

Determination of Nitric Oxide Metabolites and Cytokines in Serum

Determination of nitrite and nitrate in serum was performed with Nitrate/Nitrite Colorimetric Assay kit (Cayman Chemical, Ann Arbor, USA) following manufacture's instruction 3 and 18 hours after reperfusion.

Serum IL-6 levels were determined 3 hours after transplantation with an IL-6 ELISA (R&D Systems, Wiesbaden-Nordenstadt, Germany) following manufacturer's instructions.

Functional Studies

Mechanical in vitro activity of the mid-jejunum was evaluated at 18 hours after reperfusion using smooth muscle strips of the circular muscularis as described previously (N=8 each) (Kalff J C, Schraut W H, Simmons R L, Bauer A J. Surgical manipulation of the gut elicits an intestinal muscularis inflammatory response resulting in postsurgical ileus. Ann Surg 1998; 228: 652-663). Dose response curves were generated using increasing doses of the muscarinic agonist bethanechol (0.3-300 μmol/L). The contractile response was analyzed with the ADI Chart© software and the contractions were calculated as grams per square millimeter per second by conversion of the weight and length of the strip to square millimeters of tissue.

Determination of Apoptosis

Apoptotic cells within the smooth muscle layer of the grafts were detected 3 and 18 h after reperfusion by detection of DNA double strand breaks with a commercially available TUNEL kit (Roche, Mannheim, Germany) according to the manufactures instructions.

### Example 1

### p38-MAPK Activation

MAPK are known to play a pivotal role in transmission of proinflammatory and mechanical stress stimuli in various diseases. In postoperative ileus, a mechanical trauma initiates a massive inflammatory reaction within the muscularis externa (ME) of the intestinal wall. The activation of different MAPK pathways following abdominal surgery and intestinal manipulation (IM) was tested.

Activation of p38-MAPK by phosphorylation was detected 15 minutes after IM in ME lysates, attenuating 30 minutes postoperatively and declining to near-control levels after 1 hour (FIG. 2A). Phosphorylation of JNK/SAPK was also observed 15 minutes after IM, prolonged up to 30 minutes and showed a slight attenuation after 60 minutes. Next we analyzed the effect of preoperative intravenous CNI-1493 (5 mg/kg) administration on activation of p38-MAPK and JNK/SAPK in the ME (FIG. 2B). In CNI-1493 treated animals, upregulation of p38-MAPK phosphorylation by IM was reduced near to control levels 30 minutes after operation compared to the placebo group. JNK/SAPK phosphorylation was not affected by CNI-1493 (5 mg/kg) treatment.

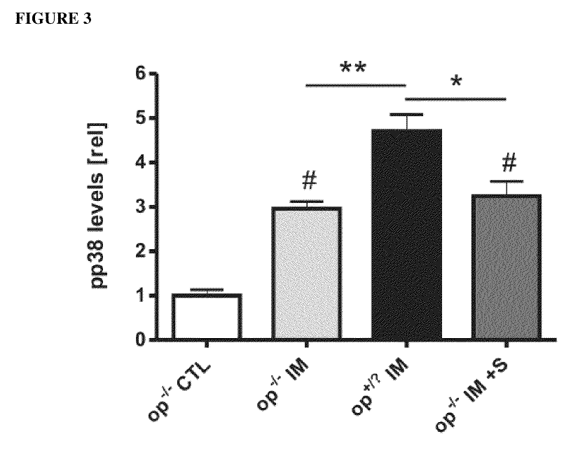

To investigate if macrophages contribute to the p38-MAPK activation we also analyzed ME lysates of op−/− mice and op+/? 20 minutes after IM (FIG. 3). Elevated levels of phopsho-p38-MAPK were observed in the macrophage-poor op−/− mice (2.96±0.16) after IM, but were significantly diminished compared to op+/? mice (4.70±0.93). Preoperative treatment of op−/− mice with CNI-1493 (5 mg/kg) i.v. did not further decrease p38-MAPK phosphorylation levels (3.24±0.66).

These results indicate that (1) p38-MAPK is immediately and strongly activated in the ME after intestinal manipulation and that (2) a guanylhydrazone is specific to macrophages, i.e. guanylhydrazone inhibits this activation exclusively in (resident muscularis) macrophages. The experiments in op−/− mice show that the p38-MAPK activation partially takes place in macrophages. These mice are nearly totally lacking macrophages in the ME due to a mutation in the CSF-1 gene. After IM, a significantly lower p38-MAPK phosphorylation could be observed in these mutant mice. The guanylhydrazone could not additionally decrease the phosphorylation, demonstrating its macrophage specificity.

Another of the proinflammatory signaling pathways is mediated via JNK/SAPK. The results indicate that this pathway was also induced immediately after the surgical trauma. Although Semapimod has also been described previously to reduce JNK/SAPK phosphorylation (Lowenberg M, Verhaar A, van den B B et al. Specific inhibition of c-Raf activity by semapimod induces clinical remission in severe Crohn's disease. J Immunol 2005; 175:2293-2300), these results could not be confirmed in the POI model. This may be a consequence of a different signalling pattern in the primary resident muscularis macrophages compared to v-myc immortalized spleen derived macrophage cell line, as used by Lowenberg et al.

### Example 2

### Proinflammatory Gene Expression

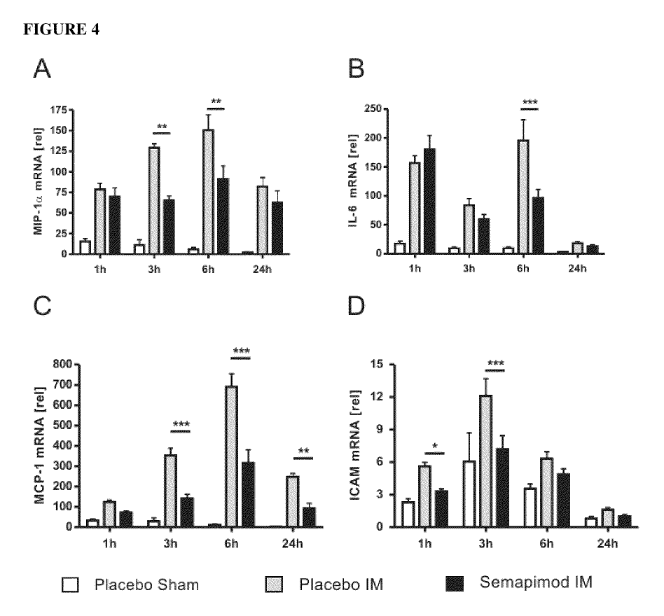

After demonstrating that CNI-1493 inhibits p38-MAPK activation immediately after IM, the present inventors analyzed the expression of proinflammatory genes that contribute to the different stages of muscularis inflammation: MIP-1α, IL-6, MCP-1 and ICAM-1. FIG. 4a demonstrates that expression of MIP-1α mRNA, a common marker for macrophage activation, is upregulated 78±20-fold after 1 hour and rises up to 150±42 fold at 6 hours after IM. Preoperative treatment with 5 mg/kg i.v. CNI-1493 resulted in a significant reduction of MIP-1α expression at 3 and 6 hours (50% and 39%, respectively). IL-6 expression also peaked at 6 hours after IM with a 214±82-fold upregulation and is significantly diminished by more than 55% in the CNI-1493 group (FIG. 4b). MCP-1 mRNA is upregulated 123±25-fold at 1 hour after IM, 353±78-fold at 3 hours, peaked with a 691±154-fold at 6 hours and decreased to 247±51-fold at 24 hours (FIG. 4c). CNI-1493 treatment resulted also in significantly diminished MCP-1 expression at 3, 6 and 24 hours (142±45, 317±143 and 92±44 respectively). As shown in FIG. 4d, ICAM-1 mRNA was also upregulated early in the placebo IM group, and significantly reduced by CNI-1493 treatment at 1 hour (5.6±0.97 vs. 3.3±0.70-fold) and 3 hours (12.1±3.5 vs. 7.2±2.8-fold).

These results demonstrate that CNI-1493 significantly diminished the proinflammatory gene expression in both, the initial and effector phase of ME inflammation.

### Example 3

### Cellular Infiltration—CNI-1493

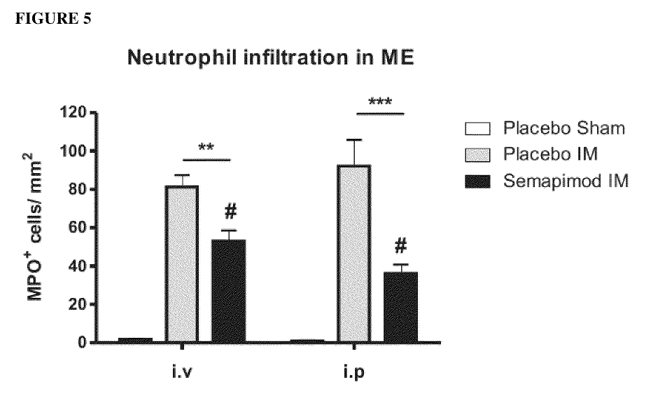

To investigate whether the reduction in proinflammatory gene expression also resulted in an effective reduction of the inflammation itself, we analyzed the infiltration of neutrophils within the ME after preoperative i.v. or i.p. application of the drug (FIG. 5). Infiltration was observed 24 hours after sham operation or IM. Placebo treatment in both, the i.v. and i.p. groups resulted in significantly increased number of cells (81.3±15.1 and 92.2±30.5) compared to sham operation (1.7±1.0 and 1.0±0.7). CNI-1493 treatment resulted in a significant reduction of neutrophils (52.1±15.5 i.v. and 36.1±10.7 i.p.) compared to the placebo IM group, but was still significantly increased compared to sham operated controls. Neutrophil reduction was higher in the i.p. treated group compared to the i.v. route, however, statistically not significantly decreased.

### Example 4

### Nitric Oxide Release from Cultured ME

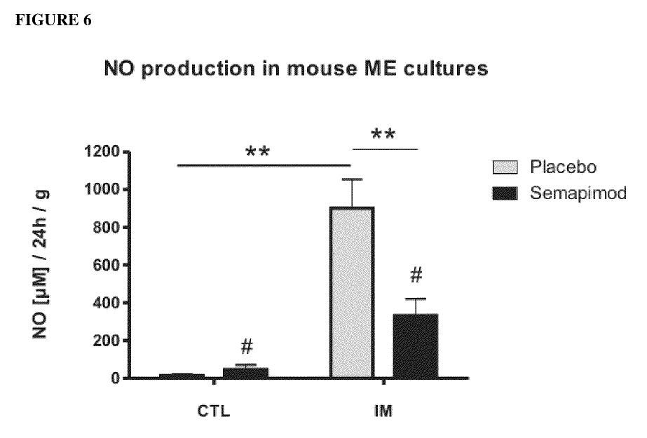

Nitric oxide (NO) is the major inhibitory neurotransmitter in the intestine. NO production was indirectly determined by the detection of nitrite release in supernatants from small intestinal muscle specimen cultures by the Griess reaction. As shown in FIG. 6, non-operated placebo (15.0±13.7 μM) and CNI-1493 treated (45.5±52.2 μM) animals did not differ significantly in their basal NO production. IM resulted in a significant increase of NO production from ME of placebo treated animal (901±306 μM), whereas in CNI-1493 (5 mg/kg) treated animals it was significantly diminished (333±198 μM). NO production in the CNI-1493 IM group did not differ significantly from both, placebo IM group and non-operated control group.

Unexpectedly, the suppression of smooth muscle contractility and gastrointestinal motility (see below) was completely abrogated by intravenous CNI-1493 treatment, but neutrophil infiltration was reduced by only one third. NO is the most important inhibitory neurotransmitter in the intestine and is responsible for the smooth muscle dysfunction in POI (Kalff J C, Schraut W H, Billiar T R et al. Role of inducible nitric oxide synthase in postoperative intestinal smooth muscle dysfunction in rodents. Gastroenterology 2000; 118:316-327; Eskandari M K, Kalff J C, Billiar T R et al. LPS-induced muscularis macrophage nitric oxide suppresses rat jejunal circular muscle activity. Am J Physiol 1999; 277:G478-G486). Unexpectedly, the NO production in CNI-1493 treated animals was nearly completely abrogated, which may explain why CNI-1493 completely prevents smooth muscle dysfunction and POI, although neutrophil infiltration is still elevated.

### Example 5

### Muscle Function

The degree of muscular dysfunction was analyzed in the following experiments by measurement of in vivo gastrointestinal transit and in vitro muscle contractility. Both methods have been proven highly reliable in previous investigations using different strains of rodents (Kalff J C, Schraut W H, Simmons R L et al. Surgical manipulation of the gut elicits an intestinal muscularis inflammatory response resulting in postsurgical ileus. Ann Surg 1998; 228:652-663; Wehner S, Behrendt F F, Lyutenski B N et al. Inhibition of macrophage function prevents intestinal inflammation and postoperative ileus in rodents. Gut 2007; 56:176-185; Wehner S, Schwarz N T, Hundsdoerfer R et al. Induction of IL-6 within the rodent intestinal muscularis after intestinal surgical stress. Surgery 2005; 137:436-446)).

In Vitro Contractility

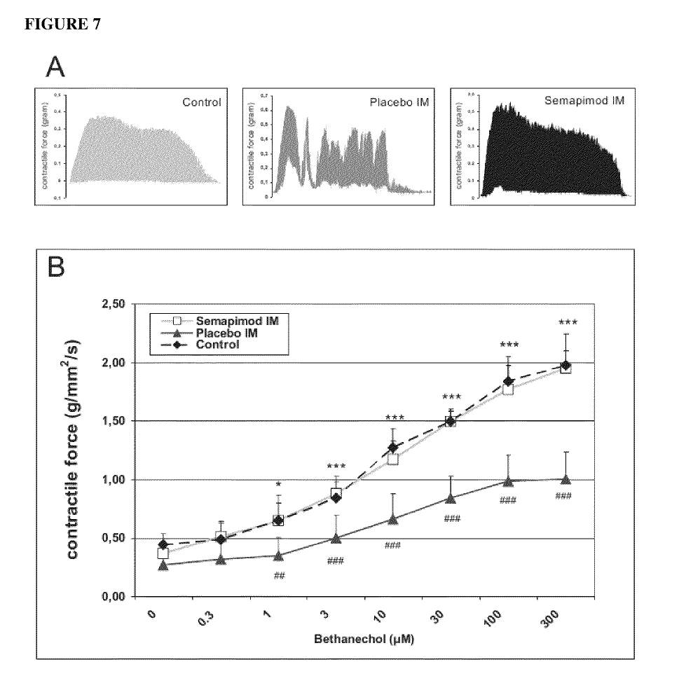

Jejunal circular smooth muscle (mouse) specimens were analyzed for spontaneous and bethanechol stimulated contractions (FIG. 7). Baseline activity of all groups did not differ significantly. Stimulation of control muscle strips (animals that did not undergo surgery) with bethanechol (0.3±300 μM) caused a dose-dependent increase in the generation of large phasic contractions. FIG. 7a shows representative contractility tracings of all groups at 100 μM bethanecol stimulation. After IM, placebo group muscle contractility was significantly decreased compared to controls at all bethanechol concentrations except 0.3 μM (FIG. 7b). In CNI-1493 (5 mg/kg) treated animals this suppression was completely abrogated and contractile force was at the level of control animals. Recorded contractions were normalized to muscle weight and length as described above.

In Vivo Gastrointestinal Transit (GIT)

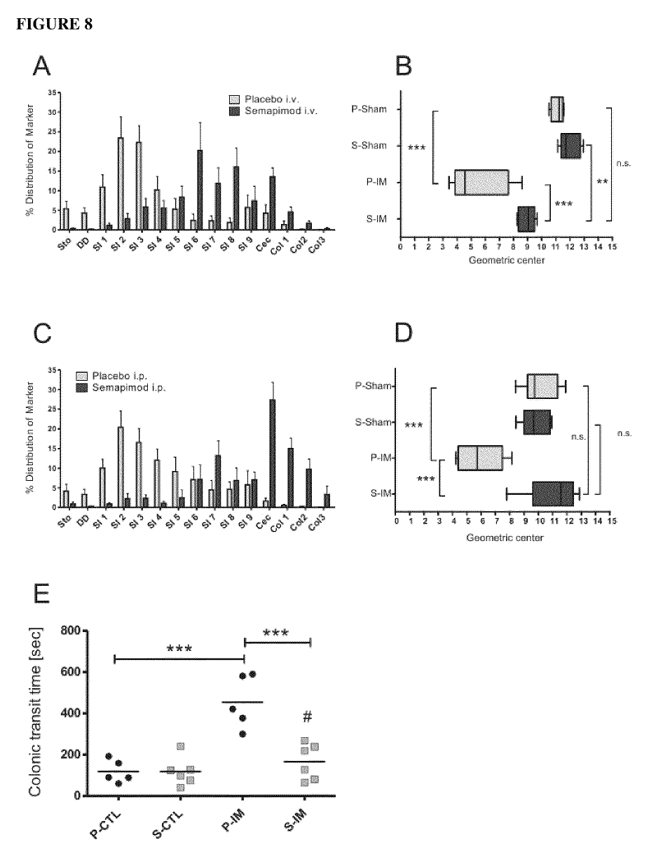

Determination of GIT time is one of the most obvious parameter for detection and quantification of intestinal dysmotility or ileus. FIG. 8 shows the distribution of a fluorescent marker along the gastrointestinal tract and calculation of the geometric center (GC) 25.5 hours after IM. Preoperatively, placebo or CNI-1493 (5 mg/kg) were administered to mice via i.v. (FIG. 8a,b) or i.p. (FIG. 8 c,d) route. FIG. 8b demonstrates that placebo i.v. treated mice had a delayed GIT following IM with a GC of 5.44±2.00 versus sham operated placebo group (GC=11.13±0.45). CNI-1493 i.v. treatment resulted in a normalized GIT (GC=8.97±0.60), significantly differing from the placebo IM group (p<0.001) but also from CNI-1493 treated sham operated animals (11.98±0.73). However, CNI-1493 IM groups did not significantly differ between i.v. and i.p. administration. For the i.p. route, FIG. 8d shows that sham operated control groups had a normal GIT after placebo (GC=10.09±1.20) or CNI-1493 treatment (GC=9.81±0.92). While IM resulted in a significant delay in the placebo group (GC=5.97±1.54), CNI-1493 treated animals had a normalized GIT (GC=10.96±1.71), not differing significantly from sham animals. Interestingly, compared to these results (where CNI-1493 was administered intravenously 90 minutes before operation), GIT was not improved when it was given immediately before laparotomy and IM (GC 5.48±2.01 CNI-1493 vs. 5.77±1.37 placebo group, results not shown).

In Vivo Colonic Transit

Although, GIT measurement demonstrated a significant improvement of the intestinal motility, it is mostly limited to the small bowel. Therefore, colonic transit time 24 hours after intestinal manipulation was also investigated. As shown in FIG. 8E, in the placebo treated groups, IM led to a significant delay compared to unoperated controls (454±128 vs. 118±55 seconds, respectively). Intravenously CNI-1493 (5 mg/kg) treated animals (mice) had a significantly improved transit time (166±87 seconds) and did not differ from unoperated controls (118±68 seconds).

Thus, colonic transit time, a clinically important factor in POI, is also reduced by CNI-1493 treatment after small bowel IM.

Intestinal Anastomotic healing

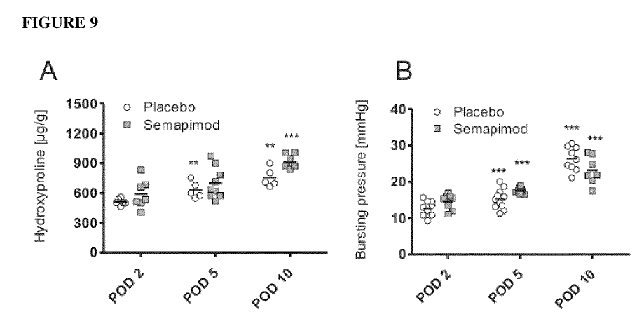

Macrophage function is known to be critical in wound healing, also in the intestine. To investigate if CNI-1493 affects intestinal anastomotic wound healing, anastomotic hydroxyproline content and bursting pressure of colonic anastomosis on POD 2, 5 and 10 in mice were analyzed. FIG. 9A demonstrates that hydroxyproline content increased from POD 2 to 10 in both, placebo and CNI-1493 (5 mg/kg) treated group. Correspondingly, anastomotic bursting increased significantly at POD 5 and 10 (FIG. 9B). However, placebo and CNI-1493 groups did not differ at any time in both experiments. This indicates that anastomotic strength and wound healing is not affected by a preoperative CNI-1493 treatment.

Thus, the results demonstrate that the collagen content, as the most prominent matrix protein mediating wound strength is not altered by CNI-1493 anastomotic strength within the first ten postoperative days. More important, also anastomotic bursting strength was not diminished by CNI-1493.

### Example 6

### Cellular Infiltration (CPSI-2364)

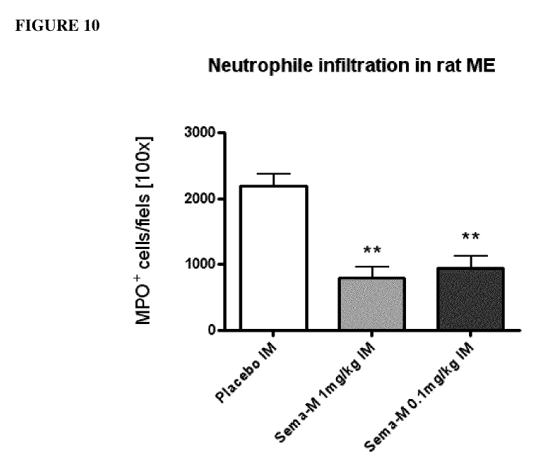

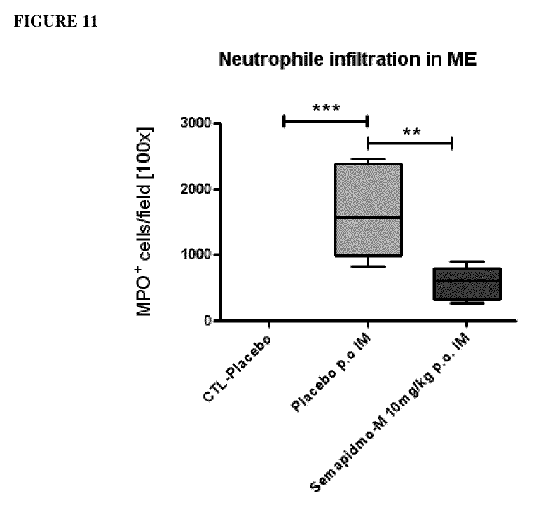

Example 3 was repeated in rats and mice, i.e. the infiltration of neutrophils within the ME after preoperative p.o. application of the drug was analyzed (FIGS. 10 and 11). However, in this example CNI-1493 was exchanged for CPSI-2364 and the route of administration was changed to p.o. FIG. 10 shows the results for rats, while FIG. 11 shows the results for mice. The drugs were administered in amounts as indicated in FIGS. 10 and 11. Infiltration was observed 24 hours after IM.

As with CNI-1493 (Example 3), placebo treatment resulted in significantly increased number of cells and CPSI-2364 treatment resulted in a significant reduction of neutrophils compared to the placebo IM group. Furthermore, even the extremely reduced amount of 0.1 mg/kg bodyweight of CPSI-2364 (administered p.o.) led to the described results (FIG. 10).

### Example 7

### Nitric Oxide Release from Rat ME (CPSI-2364)

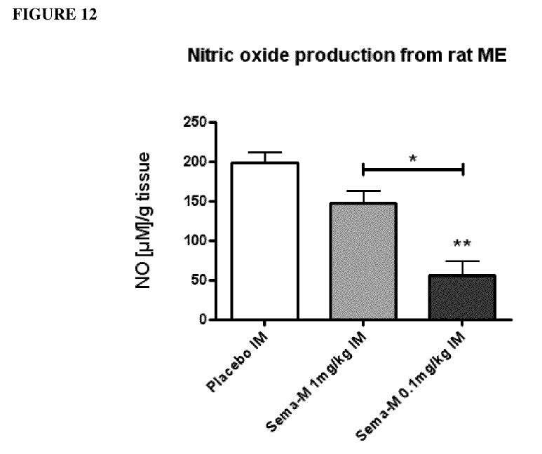

Example 4 was repeated in rats and, again, CNI-1493 was exchanged for CPSI-2364 and the route of administration was changed to p.o. The drugs were administered in amounts as indicated in FIG. 12. NO production was indirectly determined by the detection of nitrite release in supernatants from small intestinal muscle specimen cultures by the Griess reaction. As shown in FIG. 12, IM resulted in a significant increase of NO production from ME of placebo treated animal, whereas in CPSI-2364 treated animals it was significantly diminished. Again, even the extremely reduced amount of 0.1 mg/kg bodyweight of CPSI-2364 (administered p.o.) led to the described results.

### Example 8

### Muscle Function—CPSI-2364

Example 5 was repeated with CPSI-2364, instead of CNI-1493. Again, the degree of muscular dysfunction was analyzed in the following experiments by measurement of in vivo gastrointestinal transit and in vitro muscle contractility.

In Vitro Contractility

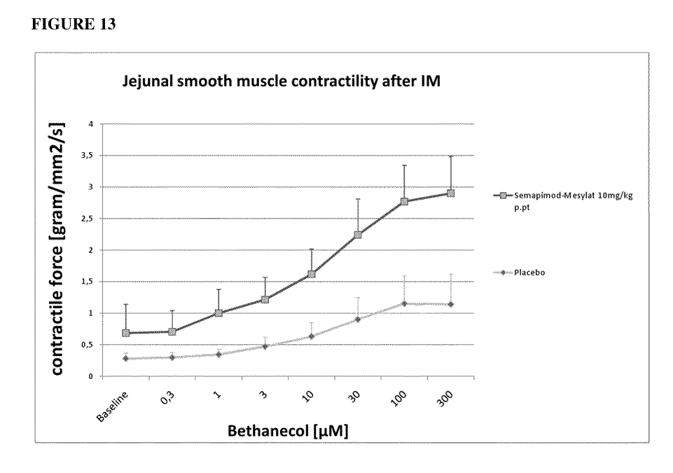

Jejunal circular smooth muscle mouse specimens were analyzed for spontaneous and bethanechol stimulated contractions (FIG. 13). Baseline activity of all groups did not differ significantly. Stimulation of control muscle strips (animals that did not undergo surgery) with bethanechol (0.3±300 μM) caused a dose-dependent increase in the generation of large phasic contractions. After IM, placebo group muscle contractility was significantly decreased compared to controls at all bethanechol concentrations except 0.3 μM (FIG. 13). In CPSI-2364 treated animals this suppression was strongly abrogated. Recorded contractions were normalized to muscle weight and length as described above.

In Vivo Gastrointestinal Transit (GIT)

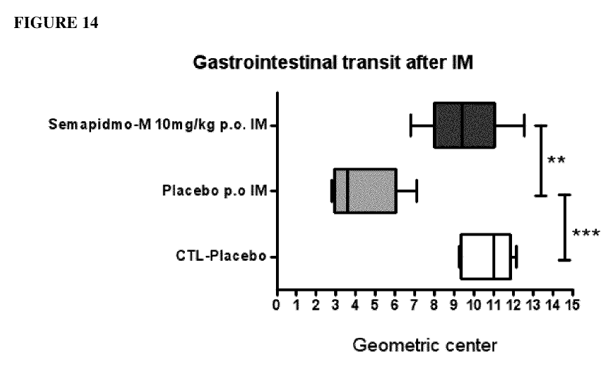

As shown in FIG. 14, IM resulted in a significant delay in the placebo group, while CPSI-2364 treated mice had a normalized GIT, not differing significantly from sham animals.

### Example 9

### Determination of Intestinal Injury (Rat Model)

Syngeneic orthotopic small intestinal transplantation was performed as described above and histological changes and intestinal injury were graded by determination of the Park score 3 and 18 hours after reperfusion. The Parks' system scores mucosal injury in intestinal villi and in crypts separately from grade 0 to 8, according to the degree or intestinal wall architecture destruction. Probes were analyzed in a blinded way.

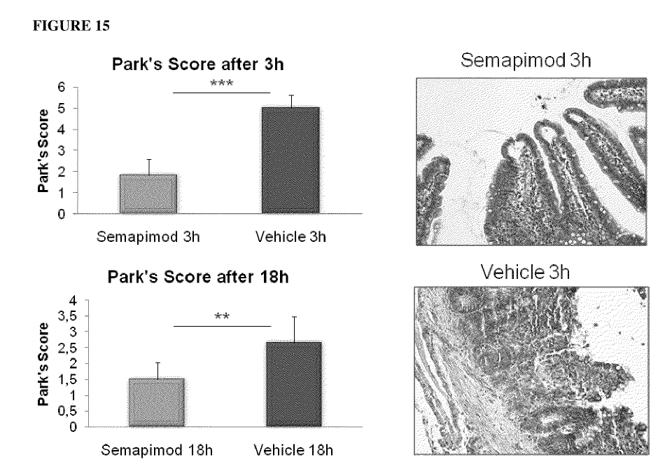

FIG. 15 shows the results of this experiment. The evaluation of histological changes between the CPSI-2364 treated animals and the vehicle treated animals revealed significant less destruction of the intestinal wall in the CPSI-2364 animals after 3 hours (Park's score: 1.83 vs. 5) and 18 hours (Park's score: 1.5 vs. 2.7).

### Example 10

### Quantification of Leukocyte Infiltration and Monocyte/Macrophage Infiltration (Rat Model)

In order to evaluate cell populations which are known to be associated with inflammation processes and to initiate immunological processes within the muscularis, leukocyte infiltrates were evaluated after 18 h by MPO histochemistry (polymorphonuclear neutrophils) and ED1 immunohistochemistry (monocytes and passenger macrophages). Specimens for histochemical analysis were prepared 18 h after operation and performed on whole mounts of the distal jejunum (n=8 each group). Whole mount preparation were fixed with 100% ethanol for 10 minutes or 4% PFA for 30 min at room temperature.

For detection of myeloperoxidase positive neutrophils (MPO+), freshly prepared whole mounts were stained with Hanker-Yates reagent as described previously (Wehner S, Schwarz N T, Hundsdoerfer R et al. Induction of IL-6 within the rodent intestinal muscularis after intestinal surgical stress. Surgery 2005; 137: 436-446). Infiltrated monocyte and immature macrophages were stained with primary anti CD68 antibody (Serotec clone ED1, Germany, 1:200 at 4° C. over night) and secondary donkey anti mouse-Cy3 (Dianova, Heidelberg, Germany, 1:200). ED1 and MPO+-cells were counted under a microscope (TE-2000, Nikon, Duesseldorf, Germany) in 5 randomly chosen areas in each specimen.

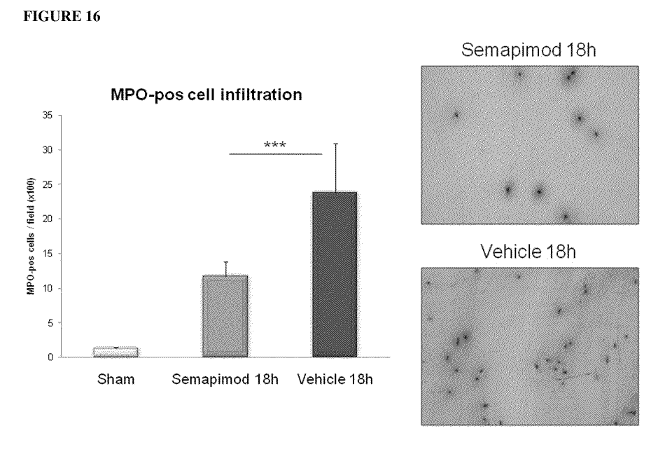

As shown in FIG. 16, a significant infiltration and recruitment of MPO-positive neutrophils, was observed in muscularis whole mounts of vehicle grafts compared to CPSI-2364 treated grafts after 18 h reperfusion (vehicle: 23.8 cells/field of view (magnification 100×), CPSI-2364.

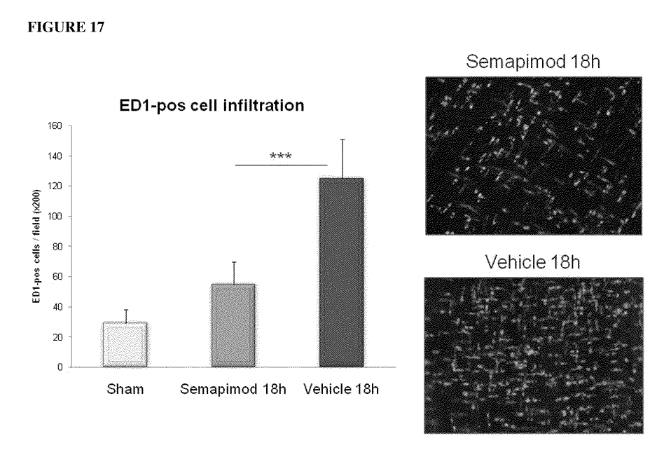

As shown in FIG. 17, evaluation of monocyte and macrophage infiltration revealed significant infiltration of ED1 positive cells in the vehicle treated muscularis (vehicle: 124.9 cells/field of view (magnification 200×) after 18 h compared to CPSI-2364 treated muscularis (CPSI-2364: 54.8).

### Example 11

### Determination of Nitric Oxide Metabolites and Cytokines in Serum (Rat Model)

Determination of nitrite and nitrate in serum was performed with Nitrate/Nitrite Colorimetric Assay kit (Cayman Chemical, Ann Arbor, USA) following manufacture's instruction 3 and 18 hours after reperfusion.

Serum IL-6 levels were determined 3 hours after transplantation with an IL-6 ELISA (R&D Systems, Wiesbaden-Nordenstadt, Germany) following manufacturer's instructions.

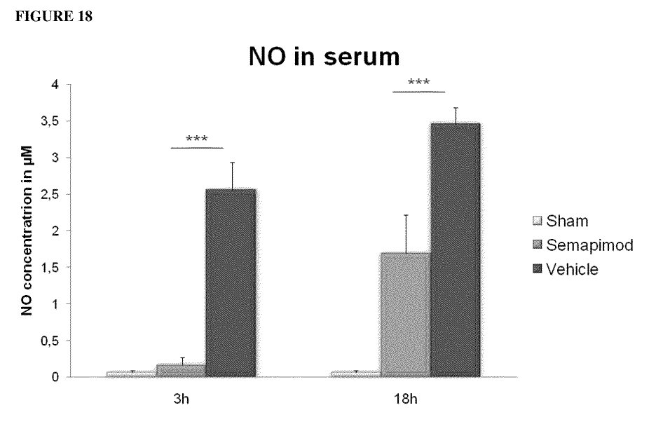

As shown in FIG. 18, the release of NO in serum of native controls as well as CPSI-2364 and vehicle treated grafts after 3 hours and 18 hours was evaluated by a Nitrate/Nitrite Colorimetric Assay kit. Nitric oxide levels within the serum were significantly increased in vehicle grafts (3 hours: 2.55 μmol/L); 18 hours: 3.45 μmol/L) compared with semapimod treated grafts (3 hours: 0.17 μmol/L); 18 hours: 1.69 μmol/L) at both time points.

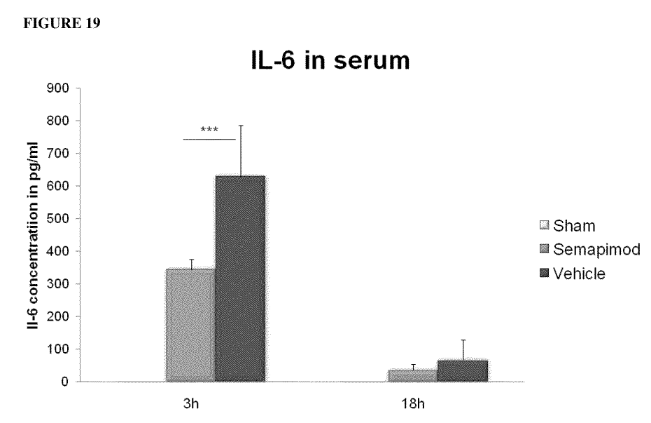

As shown in FIG. 19, release of the proinflammatory cytokine IL-6 was measured in a time course study comparing control, vehicle treated and CPSI-2364 treated grafts. Ischemia and reperfusion in vehicle treated grafts compared to semapimod treated grafts resulted in a significant higher release of IL-6 after 3 hours (Vehicle: 629 μg/ml; Semapimod: 345 pg/ml). After 18 hours no significant differences were found in between the two groups (Vehicle: 66 pg/ml; Semapimod: 35 pg/ml).

### Example 12

### Functional Studies (Rat Model)

Mechanical in vitro activity of the mid-jejunum was evaluated at 18 hours after reperfusion using smooth muscle strips of the circular muscularis as described previously (N=8 each) (Kalff J C, Schraut W H, Simmons R L, Bauer A J. Surgical manipulation of the gut elicits an intestinal muscularis inflammatory response resulting in postsurgical ileus. Ann Surg 1998; 228: 652-663). Dose response curves were generated using increasing doses of the muscarinic agonist bethanechol (0.3-300 μmol/L). The contractile response was analyzed with the ADI Chart© software and the contractions were calculated as grams per square millimeter per second by conversion of the weight and length of the strip to square millimeters of tissue.

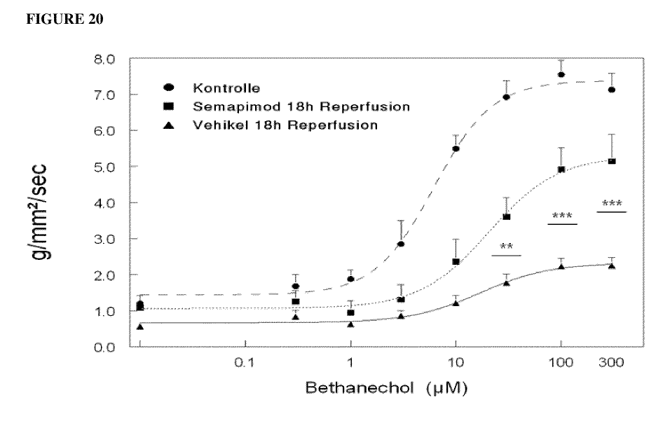

As shown in FIG. 20, the vehicle treated grafts showed a severe 79% reduction of the contractile response to 100 μmol/L bethanechol in smooth muscle contractility after 18 h reperfusion (1.85 grams/mm2/sec) compared to contractile force of naïve control muscle (8.76 grams/mm2/sec) at 100 μmol/L bethanechol. In contrast, the CPSI-2364 treated grafts (1 mg/kg, i.v. 90 min. before reperfusion) exhibited a 96% increase (3.63 grams/mm2/sec) in circular smooth muscle contractile activity in comparison to the vehicle grafts.

### Example 13

### Determination of Apoptosis (Rat Model)

Apoptotic cells within the smooth muscle layer of the grafts were detected 3 and 18 h after reperfusion by detection of DNA double strand breaks with a commercially available TUNEL kit (Roche, Mannheim, Germany) according to the manufactures instructions.

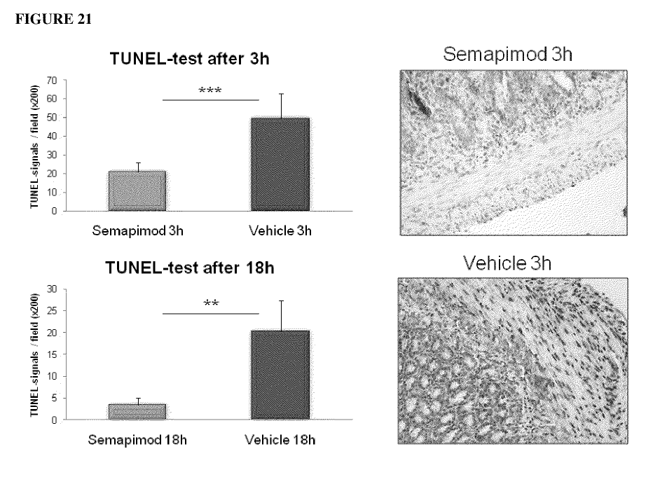

As shown in FIG. 21, analysis of apoptosis of the muscularis 3 h and 18 h after ischemia and reperfusion revealed a distinct increase in the number of apoptotic bodies (3 h: 49.5; 18 h: 14.0) for the vehicle treated grafts in comparison to CPSI-2364 treated grafts (3 h: 21.0; 18 h: 7.2)after 3 hours and 18 hours.

### Example 14

### Determination of Contractility (CPSI-2364)

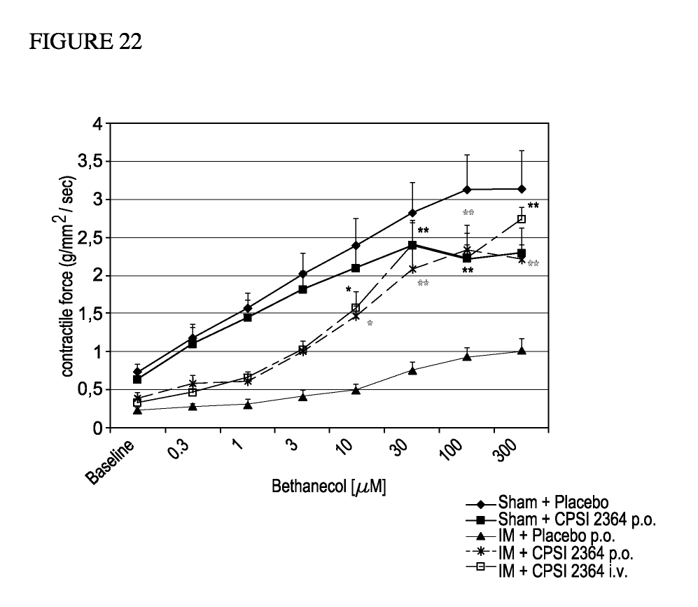

Male C57BL6/J mice (20 g) underwent intestinal manipulation (IM) or laparotomy without IM (Sham). CPSI-2364 (10 mg/kg) or placebo (mannitol 2.5%) were administered orally (p.o.) or intravenously (i.v.) 90 or 60 minutes before operation, respectively. Twenty-four hours after operation animals were sacrificed. Jejunal circular smooth muscle strips (5-6 per animal) were prepared and contractility was measured under increasing concentrations of bethanecol in an in vitro organ bath setting. n=5-6 animals per group. *p<0.05**p<0.01 vs. IM+placebo p.o by 1-way ANOVA followed by Dunnett's post test. The results are presented in FIG. 22.

### Example 15

### PMN in Mice (CPSI-2364)

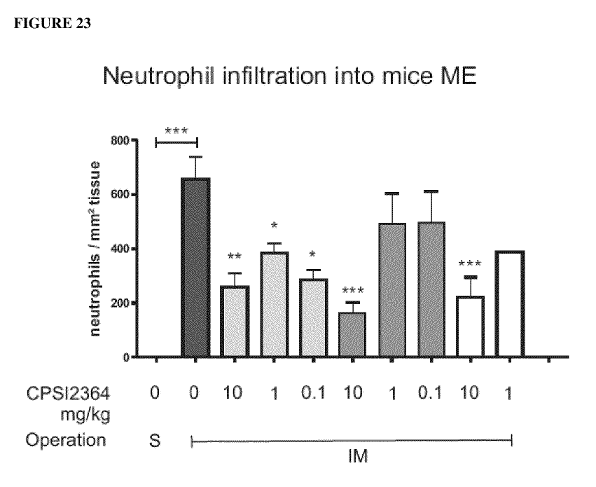

Male C57BL6/J mice (20 g) underwent intestinal manipulation (IM) or laparotomy without IM (Sham). CPSI-2364 (0.1 or 10 mg/kg) or placebo (0 mg/kg) were administered orally 90 minutes (light gray bars), 6 hours (grey bars) or 16 h (white bars) before operation. Twenty-four hours after operation animals were sacrificed, muscularis. Muscularis whole mount specimen were prepared and stained by Hanker-Yates reagent to detect myeloperoxidase positive neutrophils. Values indicate number of neutrophils per mm² tissue. p<0.05**p<0.01, ***p<0.001 vs. IM+placebo or indicated probes by 1-way ANOVA followed by Bonferroni's post test (n=3-8 per group). The results are presented in FIG. 23.

### Example 16

### Bacterial Translocation in Mice (CPSI-2364)

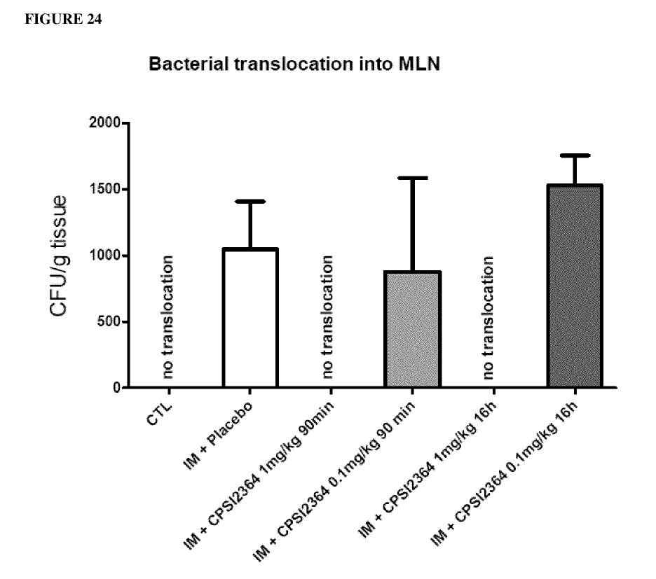

Male C57BL6/J mice (20 g) underwent intestinal manipulation (IM). Control (CTL) mice were untreated. CPSI-2364 (0.1 or 10 mg/kg) or placebo (2.5% mannitol)) were administered orally 90 minutes or 16 h hours before operation by gavage. Twenty-four hours after operation animals were sacrificed and muscularis mesenteric lymph nodes (MLN) were prepared. MLN were weighted, mechanically disrupted and dissociated in 2 ml of 3% thioglycollate medium. 5000 were plated on McConkey agar plates and incubated at 37° C. for 18 hours. Colonies (CFU) were counted and normalized to tissue weight. (n=4-8 per group). The results are presented in FIG. 24.

### Example 17

### GIT in Mice (CPSI-2364)

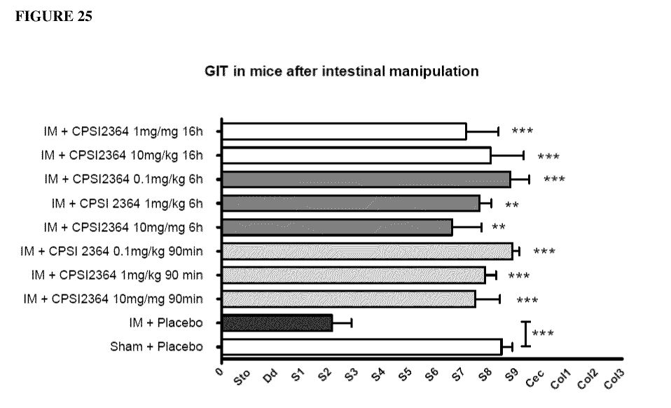

Male C57BL6/J mice (20 g) underwent intestinal manipulation (IM) or laparotomy without IM (Sham). CPSI-2364 (0.1, 1 or 10 mg/kg) or placebo (2.5% mannitol) were administered orally 90 minutes (light gray bars), 6 hours (grey bars) or 16 h (white bars) before operation. Twenty-four hours after operation animals were fed with 2000 of a FITC dextran solution by gavage. 90 minutes later animals were sacrificed, the complete gastrointestinal tract was removed and divided in 15 parts (Sto=Stomach, Dd, =duodenum, S1-S9=small bowel segments, Cec=Cecum, Coll-3=Colon segments). FITC dextran contents were determined in each segment by fluorometric measurement. Values indicate the geometric centers of FITC dextran distribution. None of the CPSI-2364 treated groups differed significantly from the Sham+Placebo group. **p<0.01, ***p<0.001 vs. IM+placebo or indicated probes by 1-way ANOVA followed by Dunnett's post test (n=3-10 per group). The results are presented in FIG. 25.

### Example 18

### Colonic Transit in Mice (CPSI-2364)

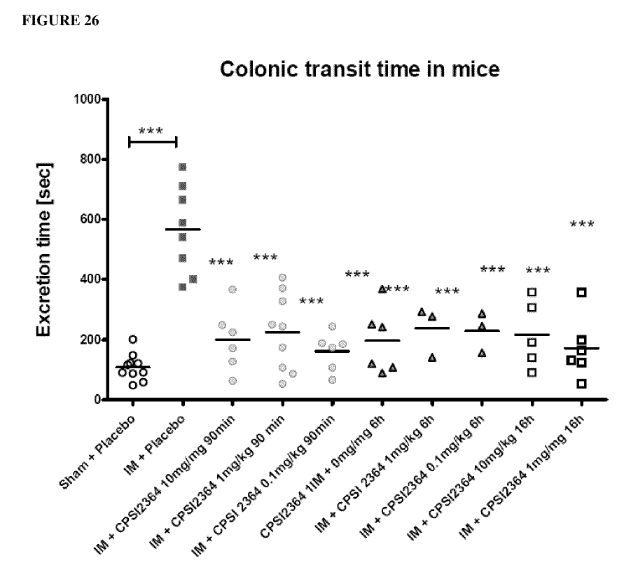

Male C57BL6/J mice (20 g) underwent intestinal manipulation (IM) or laparotomy without IM (Sham). CPSI-2364 (0.1, 1 or 10 mg/kg) or placebo (2.5% mannitol) were administered orally 90 minutes (light gray circles), 6 hours (grey triangles) or 16 h (white squares) before operation. Twenty-four hours after operation a 2 mm glass ball was inserted by a metal rod 3 cm into the colon. Excretion time of the ball was measured in seconds. None of the CPSI-2364 treated group differed significantly from Sham+Placebo group. ***p<0.001 vs. IM+placebo or indicated probes by 1-way ANOVA followed by Bonferroni's post test (n=3-11 per group). The results are presented in FIG. 26.

### Example 19

### Nitric Oxide in Mice (CPSI-2364)

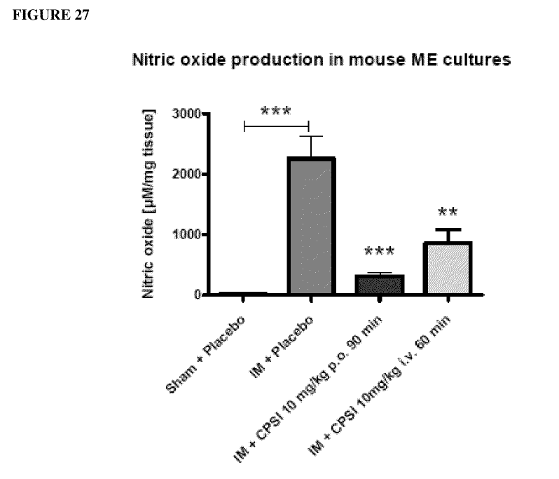

Male C57BL6/J mice (20 g) underwent intestinal manipulation (IM) or laparotomy without IM (Sham). CPSI-2364 (10 mg/kg) or placebo (2.5% mannitol) were administered orally 90 minutes or intravenously 60 minutes before operation. Twenty-four hours after operation small bowel muscularis was prepared and cultured for additional 24 hours in 1 ml DMEM culture medium. Cell-free culture supernatant was analyzed for nitric oxide and its metabolites by Griess reaction. Values were normalized by tissue weight. None of the CPSI-2364 treated group differed significantly different from Sham+Placebo group. **<p0.01 and ***p<0.001 vs. IM+placebo or indicated probes by 1-way ANOVA followed by Bonferroni's post test (n=4-5 per group). The results are presented in FIG. 27.

SWINE Model

Swine were fed with CPSI 2364 or placebo 14 hours and three hours before operation. In the iv group, CPSI 2364 was applied once two hours before operation. All animals were fed (non fasted). After premedication animals were intubated and a central venous catheter was applied. A laparotomy was performed under sterile conditions. The whole small bowel was eventrated and manipulated twice from duodenum to cecum between two fingers. In the sham group, a laparotomy was performed without intestinal manipulation. At the end of the operation, 15 radio-opaque globes were placed in the proximal small bowel. The abdominal wall was closed and swine were brought to an intensive care unit.

### Example 20

### Defecation CPSI-2364 Swine

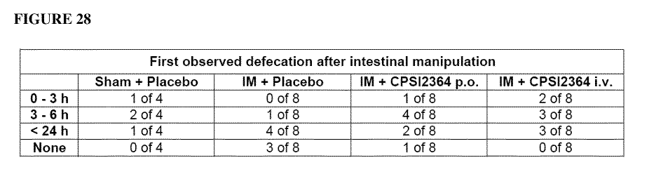

Swine underwent intestinal manipulation (IM) or laparotomy without IM (Sham). CPSI-2364 (1 mg/kg) or placebo (mannitol 2.5%) were administered orally (p.o.) twice at 14 hours and 3 hours before operation or intravenously (i.v.) once at 2 hours before operation. Three, 6 and 24 hours after operation animals were examined for first defecation. The results are presented in FIG. 28.

### Example 21

### Contractility CPSI-2364 Swine

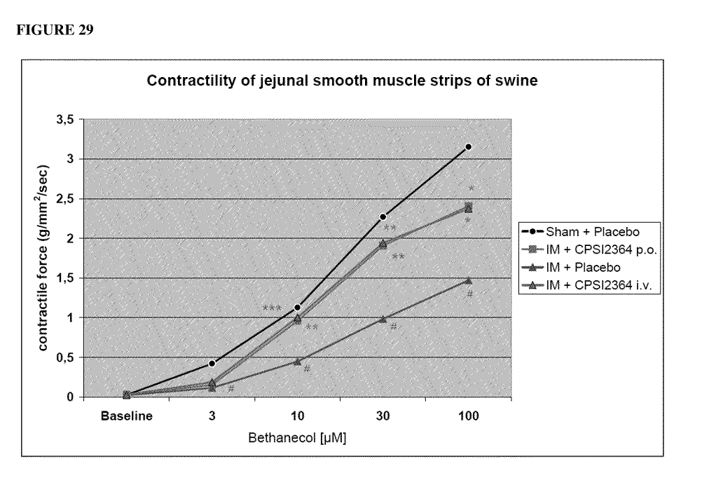

Swine underwent intestinal manipulation (IM) or laparotomy without IM (Sham). CPSI-2364 (1 mg/kg) or placebo (mannitol 2.5%) were administered orally (p.o.) twice at 14 hours and 3 hours before operation or intravenously (i.v.) once at 2 hours before operation. Twenty-four hours after operation animals were sacrificed. Jejunal circular smooth muscle strips were prepared and contractility was measured under increasing concentrations of bethanecol in an in vitro organ bath setting. n=5-6 animals per group. *p<0.05**p<0.01 vs. IM+Placebo p.o by 1-way ANOVA followed by Bonferroni's post test. The results are presented in FIG. 29.

### Example 22

### Anastomoses CPSI-2364 Swine

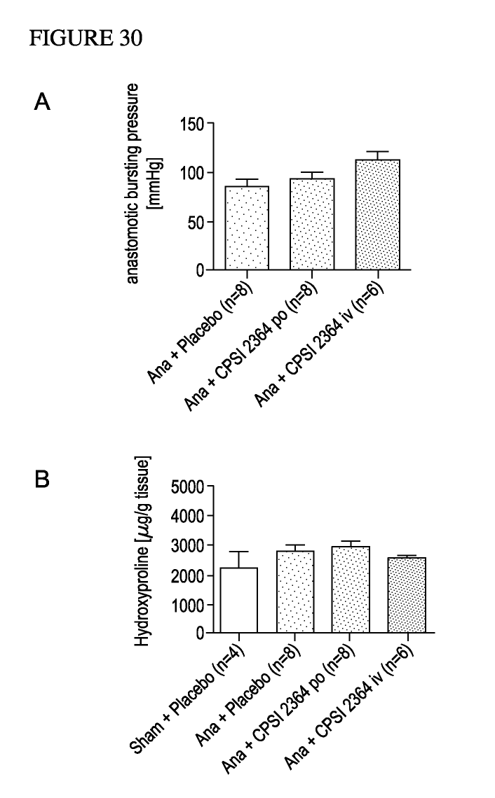

Swine were fed with 1 mg/kg CPSI-2364 or placebo (2.5% mannitol) 14 hours and 3 hours before operation. In the iv group, 1 mg/kg CPSI 2364 was fed once 2 hours before operation. After premedication animals were intubated and a central venous catheter was applied. A laparotomy was performed under sterile conditions. The distal part of the colon, comparable to the human sigmoid, was identified and cut through followed by an anastomosis with continuous suture (4/0 monocryl).On postoperative day 6 (pod6) animals were sacrificed, the anastomotic region was removed (Ana) and analyzed for hydroxyproline content and its strength by determination of the bursting pressure (in mmHg). No differences were observed in the bursting pressure levels (A) and hydroxyproline content (B), indicating that preoperative oral CPSI-2364 treatment does not affect intestinal anastomotic healing. The results are presented in FIG. 30.

### Example 23

### MPO Assay CPSI-2364 Swine

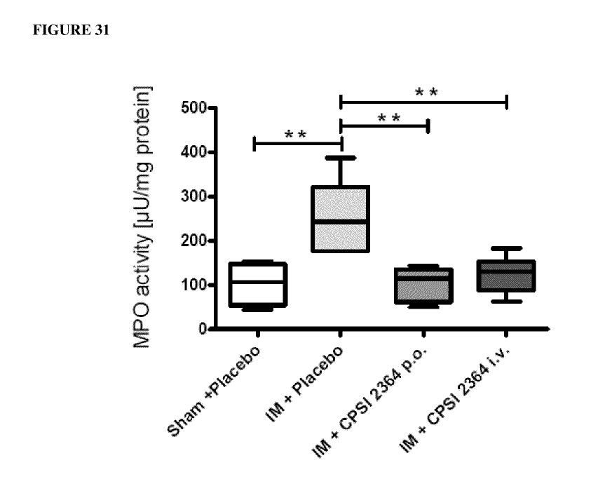

Swine underwent intestinal manipulation (IM) or laparotomy without IM (Sham). CPSI-2364 (1 mg/kg) or placebo (mannitol 2.5%) were administered orally (p.o.) twice at 14 hours and 3 hours before operation or intravenously (i.v.) once at 2 hours before operation. Twenty-four hours after operation animals were sacrificed. Muscularis specimens were prepared and myeloperoxidase activity was measured to determine the degree of neutrophil infiltration. **p<0.01 vs. indicated probes by 1-way ANOVA followed by Bonferroni's post test (n=6 per group). The results are presented in FIG. 31.

### Example 24

### MCP-1 CPSI-2364 swine

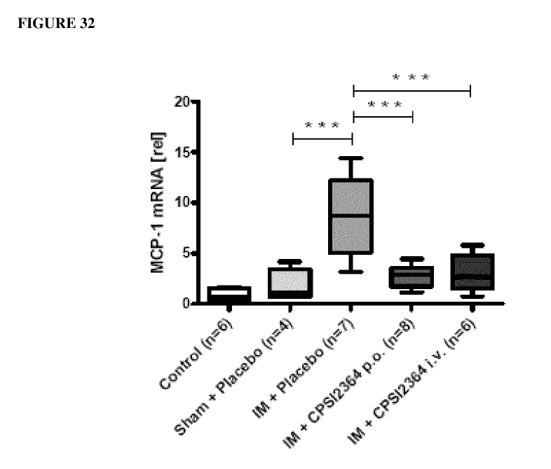

Swine underwent intestinal manipulation (IM) or laparotomy without IM (Sham). CPSI-2364 (1 mg/kg) or placebo (mannitol 2.5%) were administered orally (p.o.) twice at 14 hours and 3 hours before operation or intravenously (i.v.) once at 2 hours before operation. Twenty-four hours after operation animals were sacrificed. Muscularis specimens were analyzed for macrophage chemoattractant protein −1 (MCP-1) mRNA expression by quantitative real time PCR. Expression levels were normalized to muscularis of untreated swine (control). ***p<0.001 vs. indicated probes by 1-way ANOVA followed by Bonferroni's post test. The results are presented in FIG. 32.

### Example 25

### GIT CPSI-2364 Swine

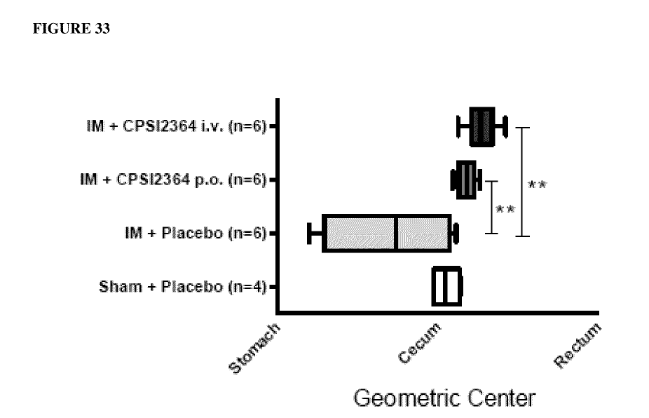

Swine underwent intestinal manipulation (IM) or laparotomy without IM (Sham). CPSI-2364 (1 mg/kg) or placebo (mannitol 2.5%) were administered orally (p.o.) twice at 14 hours and 3 hours before operation or intravenously (i.v.) once at 2 hours before operation. At the end of the operation, 15 radio-opaque globes were placed in the proximal small bowel. Twenty-four hours after operation animals were sacrificed and the radio-opaque globes were detected by x-rays radiography and the distribution along the gastrointestinal tract was calculated as the geometric center **p<0.001 vs. indicated probes by 1-way ANOVA followed by Bonferroni's post test. GC for the individual as mean+/− standard error of the mean were as follows: Sham+Placebo: 11.60+/−0.49; IM+Placebo: 7.67+/−1.74; IM+CPSI-2364 p.o.: 13.00+/−0.27; IM+CPSI-2364 i.v. 14.01+/−0.44. The results are presented in FIG. 33.

The contents of all references, published patents, and patents cited throughout the present application are hereby incorporated by reference in their entirety.

1. A method, comprising administering at least one guanylhydrazone or salt thereof or a combination thereof to a subject to prevent or ameliorate in said subject at least one of postoperative intestinal inflammation, postoperative ileus, ischemia reperfusion injury, or a combination thereof.

2. The method of claim 1, wherein the administering is carried out prior to a surgery on said subject.

3. The method of claim 1, wherein the administering is carried out prior to and during a surgery on said subject.

4. The method of claim 1, further comprising, after said administering, carrying out surgery on said subject.

5. The method of claim 1, wherein the salt is administered.

6. The method of claim 1, wherein the administering is carried out orally, intravenously, intraperitoneally, intramuscularly, subcutaneously, absorptively through epithelial or mucocutaneous lining, rectally, transdermally, topically, intradermally, intragastrally, intracutanly, intravaginally, intravasally, intranasally, intrabuccally, percutanly, sublingually, inhalatively, parenterally, or combination thereof.

7. The method of claim 1, wherein the administering is carried out intravenously, subcutaneously, intramuscularly, intraperitoneally, inhalative, orally, or a combination thereof.

8. The method of claim 1, wherein the administering is carried out orally or intravenously.

9. The method of claim 1, wherein the guanylhydrazone is a tetravalent mesylate or hydrochloride salt of the following compound:

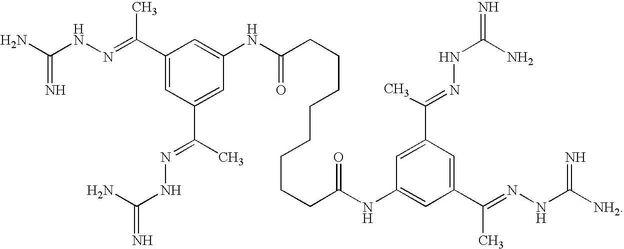

10. The method of claim 1, wherein the guanylhydrazone or salt thereof or a combination thereof is administered together with at least one pharmaceutically acceptable carrier.

11. A surgical method, comprising:
administering to a subject a guanylhydrazone or salt thereof or combination thereof; and
thereafter, performing surgery on said subject.

12. The method of claim 11, wherein the surgery comprises abdominal surgery, cardiothoracic surgery, trauma surgery, orthopedic surgery, heart surgery, thorax surgery, transplantation, open surgery, minimally invasive surgery, small bowel transplant, or a combination thereof.
